(19) United States
(12) Patent Application Publication
(10) Pub. No.: US 2009/0081237 A1
(43) Pub. Date: Mar. 26, 2009

(54) Prognostic, diagnostic, and cancer therapeutic uses of FANCI and FANCI modulating agents

(75) Inventors: Alan D. D'Andrea, Winchester, MA (US); Patrizia Vinciguerra, Boston, MA (US); Stephen J. Elledge, Brookline, MA (US); Shuhei Matsuoka, Brookline, MA (US); Agata M. Smogorzewska, Boston, MA (US); Kay O. Hofmann, Koeln (DE)

Correspondence Address: EDWARDS ANGELL PALMER & DODGE LLP, P.O. BOX 55874, BOSTON, MA, 02205, US

(73) Assignee: Dana-Farber Cancer Institute, Boston, MA (US)

(21) Appl. No.: 12/075,162

(22) Filed: Mar. 10, 2008

Related U.S. Application Data
(60) Provisional application No. 60/906,724, filed on Mar. 12, 2007.

(51) Int. Cl. A61K 39/395; C12Q 1/68; G01N 33/53; C07K 14/00; C07K 7/08; A61P 35/00; C07H 21/04; C12Q 1/02

(52) U.S. Cl. 424/172.1; 435/6; 435/29; 435/7.1; 536/23.1; 530/402

(57) ABSTRACT

Disclosed herein are methods and compositions for the treatment of cancer. In particular, the present invention identifies and characterizes the FANCI polypeptide as a vital component of the Fanconi anemia pathway and discloses inhibitors of FANCI and methods of using same. Such inhibitors are useful in inhibiting DNA damage repair and can be useful, for example, in the treatment of cancer.

### CROSS-REFERENCE TO RELATED APPLICATION

The present application is related to and claims priority under 35 U.S.C. § 119(e) to U.S. provisional patent application No. 60/906,724 filed 12 Mar. 2007, incorporated herein by reference.

### GOVERNMENT SUPPORT

This invention was made with government support under N.I.H. grants RO1-HL52725 and RO1-DK43889. The U.S. Government has certain rights in the invention.

### FIELD OF THE INVENTION

This invention generally relates to compositions and methods for the treatment of cancer.

### BACKGROUND OF THE INVENTION

The ability to sense and respond to DNA damage and DNA replication stress is critical for cellular and organismal survival. A failure to properly respond to genotoxic stress can lead to both developmental difficulties and tumorigenesis. Cells have evolved a complex signal transduction pathway that senses genotoxic stress and responds by activating specific types of repair, arresting the cell cycle and altering transcription. At the core of this signal transduction pathway are the ATM and ATR kinases (Bakkenist and Kastan (2004) Cell 118: 9-17; Bartek et al. (2004) δ: 792-804; Zhou and Elledge (2000) Nature 408: 433-439). These kinases phosphorylate over 20 known proteins in response to damage, including the Chk1 and Chk2 kinases. While early theories regarding these pathways considered their major role to be controlling cell cycle transitions, it is now clear that they play critical roles in regulating essential functions in both DNA replication and DNA repair.

One pathway regulated by ATM/ATR is the Fanconi anemia (FA) crosslink repair pathway (Gurtan and D'Andrea (2006) DNA Repair (Amst) δ: 1119-1125). Patients with FA display multi-organ defects and most develop bone marrow failure in childhood (Butturini et al. (1994) 84: 1650-1655; Fanconi (1967) Semin Hematol 4: 233-240; Schmid and Fanconi (1978) Cytogenet Cell Genet. 20: 141-149). FA patients have a high incidence of hematological and nonhematological malignancies and their cells are hypersensitive to DNA interstrand crosslinking agents such as mitomycin C (MMC) (Alter et al. (2003) Blood 101: 2072). FA falls into 13 complementation groups, and 12 FA genes have previously been cloned (Gurtan and D'Andrea (2006) DNA Repair (Amst) 5: 1119-1125; Reid et al. (2007) Nat Genet. 39: 162-164; Taniguchi and D'Andrea (2006) Blood 107: 4223-4233; Xia et al. (2007) Nat Genet. 39: 159-161; Xia et al. (2006) Mol Cell 22: 719-729). Eight of these proteins (all but D2, D1, J, and N) are subunits of a FA core complex, a nuclear E3 ubiquitin ligase (Machida et al. (2006) Mol Cell 23: 589-596; Meetei et al. (2004) Cell Cycle 3: 179-181). A key substrate of this ligase is FANCD2, which has been shown to be monoubiquitinated on lysine 561 (Garcia-Higuera et al. (2001) Mol Cell 7: 249-262). It has been hypothesized that there is another critical substrate for the ligase in addition to FANCD2 because fusion of ubiquitin to the chicken FANCD2 protein mutant for the lysine acceptor was observed to allow complementation of chicken FANCD2 mutants but not FA mutants defective for the ligase activity (Matsushita et al. (2005) Mol Cell 19: 841-847).

FANCD2 ubiquitination was identified as critical for MMC-resistance and was observed to be required for the FANCD2 protein to form damage-induced foci on chromatin (Garcia-Higuera et al. (2001) Mol Cell 7: 249-262). The mechanism by which the FA pathway controls inter-strand crosslink repair has remained unclear; however, one important finding was that the FANCD1 gene is BRCA2, which has a known role in regulation of Rad51 loading and homologous recombination (Howlett et al. (2002) Science 297: 606-609).

Of all of the FA complementation groups, only FA-I has until now remained uncharacterized at the molecular level (Levitus et al. (2004) Blood 103: 2498-2503). FA-I mutant cells were previously identified to not ubiquitinate FANCD2, precluding its localization to repair foci. Like FA-D2 cells, FA-I cell lines have been demonstrated to exhibit normal FA E3 ligase complex formation (Levitus et al. (2004) Blood 103: 2498-2503). Identification of a gene that complements FA-I mutant cells will prove advantageous for the improvement of existing therapies and development of new therapies for Fanconi anemia and also cancer, as the FA pathway has been shown to be particularly relevant to cancers that resist chemotherapeutic treatment.

Many kinds of cancer resist effective chemotherapeutic treatment. In ovarian cancer, resistance is observed towards chemotherapeutic agents such as cisplatin. Cisplatin (cis-diamminedichloroplatinum, or CDDP), discovered originally in the late 1960s, is a cytotoxic drug used to treat many cancers, including ovarian cancer. Cisplatin acts by platination of DNA, resulting in DNA crosslinking. Up to 50% of ovarian carcinomas are intrinsically resistant to conventional chemotherapeutic agents such as cisplatin or other related platinum therapies. Many mechanisms of resistance have been postulated. However, the precise mechanism(s) underlying the intrinsic and extrinsic resistance to chemotherapy has not been elucidated. One method of reversing resistance to chemotherapy involves the use of chemosensitizers. Chemosensitizers generally inhibit the mechanism of resistance. Examples include verapamil, reserpine, tamoxifen and cremophor, inhibitors of efflux pumps conferring multidrug resistance (MDR1, P-glycoprotein). However, such chemosensitizers are effective only in a subset of tumors where drug efflux is the main mechanism of resistance. In addition, a number of these chemosensitizers have undesirable side effects.

### SUMMARY OF THE INVENTION

The present invention, at least in part, is based upon the discovery and characterization of FANCI as a component the Fanconi anemia (FA) pathway. Defects in the FA pathway have been identified as critical not only to Fanconi anemia, but also in cancer predisposition. In addition, the FA pathway has been described as critical to inducing resistance to chemotherapeutic agents, e.g., cisplatin, in cancer patients. Identification of FANCI as a monoubiquitinated phosphoprotein that is closely associated with the FANC D2 protein not only provides a key marker of Fanconi anemia, neuroplasia and chemotherapeutic resistance, but also provides a critical therapeutic target. Accordingly, the instant invention, at least in part, provides for use of FANCI as a prognostic and diagnostic disease marker, a genetic marker, and as a therapeutic target for use in screening methods for agents capable of modulating FANCI activity and/or levels.

In one aspect, the invention provides a method of diagnosing or determining if a subject has cancer or is at increased risk of cancer involving testing a sample from the subject for the presence of FANCI-containing foci using an antibody specific for FANCI, with presence of FANCI-containing foci indicative of cancer or an increased risk of cancer in the subject.

In certain embodiments, the antibody or antigen binding fragment thereof is a monoclonal antibody or a polyclonal antibody. In one embodiment, the antibody or antigen binding fragment thereof is anti-KLAA1794 antibody BL999 or BL1000. In another embodiment, the antibody or antigen binding fragment thereof is detectably labeled, with the detectable label optionally a radioactive, enzymatic, biotinylated or fluorescent label.

In one embodiment, the sample is derived from heart, brain, placenta, liver, skeletal muscle, kidney, pancreas, spleen, thymus, prostate, testis, uterus, small intestine, colon, peripheral blood or lymphocytes. In another embodiment, the sample is a blood sample or biopsy sample of tissue from the subject or a cell line. In an additional embodiment, the cancer is a melanoma, leukemia, astocytoma, glioblastoma, lymphoma, glioma, Hodgkins lymphoma, chronic lymphocyte leukemia or cancer of the pancreas, breast, thyroid, ovary, uterus, testis, pituitary, kidney, stomach, esophagus or rectum.

In another aspect, the invention provides a method of diagnosing or determining if a subject has cancer or is at increased risk of cancer involving testing a FANCI gene of the subject for the presence of a cancer-associated coding change, with presence of one or more cancer-associated coding changes indicative of cancer or an increased risk of cancer in the subject.

In one embodiment, the cancer-associated coding change encodes a change in the FANCI polypeptide at K523, K1269, R1285, S730, T952, S1121, or P55. In a related embodiment, the change in the FANCI polypeptide is R1285Q.

In an additional aspect, the invention provides a method of determining if a subject has cancer, or is at increased risk of developing cancer, by providing a DNA sample from the subject, amplifying the FANCI gene from said subject with any of the FANCI gene-specific polynucleotide primers shown in Example 1, sequencing the amplified FANCI gene, and comparing the FANCI gene sequence from the subject to a reference FANCI gene sequence, where a discrepancy between the two gene sequences indicates the presence of a cancer-associated defect, with one or more such defects indicative of the subject having cancer or being at an increased risk of developing cancer.

In one embodiment, the patient has no known cancer causing defect in the BRCA 1 or BRCA-2 genes.

In a further aspect, the invention provides a method of diagnosing or determining if a subject has Fanconi anemia or is at increased risk of developing Fanconi anemia involving testing a FANCI gene of the subject for the presence of a Fanconi anemia-associated coding change, with the presence of one or more Fanconi anemia-associated coding changes indicative of Fanconi anemia or an increased risk of Fanconi anemia in the subject.

In one embodiment, the Fanconi anemia-associated coding change encodes a change in the FANCI polypeptide at K523, K1269, R1285, S730, T952, S1121, AND P55.

In another aspect, the invention provides a method of determining if a subject has cancer, or is at increased risk of developing cancer, involving providing a DNA sample from the subject, amplifying the FANCI gene from the subject with FANCI gene-specific polynucleotide primers, sequencing the amplified FANCI gene, and comparing the FANCI gene sequence from the subject to a reference FANCI gene sequence, where a discrepancy between the two gene sequences indicates the presence of a cancer-associated coding change, with presence of one or more cancer-associated coding changes indicative of cancer or an increased risk of developing cancer in the subject.

In one embodiment, the FANCI gene-specific polynucleotide primers are selected from the group consisting of SEQ ID NOs: 1-8.

In an additional aspect, the invention provides a method of predicting whether a subject with a neoplastic disorder will respond to a genotoxic anti-neoplastic agent involving determining the size or number of FANCI-containing foci in a sample from the subject using an antibody or antigen binding fragment thereof specific for FANCI, wherein if the number or size of the foci is reduced relative to the number or size of such foci in a sample from a control subject, the subject is predicted to respond to a genotoxic anti-neoplastic agent.

In one embodiment, the subject was exposed to the genotoxic anti-neoplastic agent prior to the sample being obtained from the subject. In a related embodiment, the exposure is less than or equal to a therapeutically effective dose. In another embodiment, the exposure is about 50% or less of the therapeutically effective dose. In an additional embodiment, the sample was exposed to the genotoxic anti-neoplastic agent prior to determining the number or size of foci. In a further embodiment, the genotoxic anti-neoplastic agent is selected from the group consisting of 1,3-bis(2-chloroethyl)-1-nitrosourea, busulfan, carboplatin, carmustine, chlorambucil, cisplatin, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, oxaliplatin, temozolomide, topotecan, or ionizing radiation.

In one embodiment, the number or size of said foci in a sample from the subject is less than about 70% of the number or size of said foci in a sample from a control subject.

In another aspect, the invention provides a method of predicting whether a subject with a neoplastic disorder will respond to a genotoxic anti-neoplastic agent involving determining the degree of ubiquitination of FANCI polypeptide in a sample from the subject, wherein if the degree of ubiquitination of the FANCI polypeptide in the sample is reduced when compared with a sample from a control subject, the subject is predicted to respond to a genotoxic anti-neoplastic agent.

In one embodiment, the sample was exposed to the genotoxic anti-neoplastic agent prior to determining the degree of ubiquitination of FANCI polypeptide. In another embodiment, the degree of monoubiquitination of FANCI polypeptide is determined by immunoblot analysis using an antibody or antigen binding fragment thereof specific for FANCI.

In an additional aspect, the invention provides a method of identifying a tumor that is sensitive to a genotoxic anti-neoplastic agent involving determining the size or number of FANCI-containing foci in a sample from a test subject, wherein if the number or size of foci is reduced relative to the number or size of foci in a sample from a control subject, then the sample from the test subject is identified as a tumor that is sensitive to a genotoxic anti-neoplastic agent.

In one embodiment, the sample was exposed to the genotoxic anti-neoplastic agent prior to determining the number or size of the foci.

In certain embodiments, the subject is human.

In another aspect, the invention provides a method of identifying an inhibitor of a Fanconi anemia DNA repair pathway involving contacting a cell with a test compound; before, after or simultaneously contacting the cell with a genotoxic anti-neoplastic compound; and quantifying FANCI-containing foci in the cell using an antibody or antigen binding fragment thereof specific for FANCI, wherein if the quantity of foci is less than in a control cell contacted with the genotoxic anti-neoplastic agent but not with the test compound, then the test compound is identified as an inhibitor of a Fanconi anemia DNA repair pathway.

In one embodiment, the method of the invention further comprises for a test compound identified as an inhibitor, determining the degree of monoubiquitination of FANCI polypeptide in the cell, wherein if the degree of monoubiquitination of FANCI polypeptide is less than in the control cell, then the test compound is further identified as an inhibitor of a Fanconi anemia DNA repair pathway. In a related embodiment, the method of the invention further comprises for a test compound further identified as an inhibitor, contacting a test cell that has a functional Fanconi anemia pathway with the test compound and the genotoxic anti-neoplastic agent, measuring the sensitivity of the test cell to the genotoxic anti-neoplastic agent, and comparing the sensitivity of the test cell to the agent to that of a second control cell, wherein the second control cell is isogenic to the test cell but has a defective Fanconi anemia pathway, and wherein if the sensitivity of the test cell is comparable to the sensitivity of the second control cell, the test compound is further identified as an inhibitor of a Fanconi anemia DNA repair pathway.

In one embodiment, the number of FANCI-containing foci is determined while quantifying FANCI-containing foci. In another embodiment, the size of FANCI-containing foci is determined while quantifying FANCI-containing foci. In certain embodiments, quantification of FANCI-containing foci is performed in high throughput format. In another embodiment, the degree of monoubiquiti nation of FANCI polypeptide is determined by immunoblot analysis. In an additional embodiment, the sensitivity of the test cell and the second control cell to the anti-neoplastic agent is determined by measuring cell survival at one or more concentrations of the anti-neoplastic agent. In another embodiment, the test cell and the second control cell are human cells.

In an additional aspect, the invention provides a method of identifying an inhibitor of a non-Fanconi anemia DNA repair pathway involving contacting a test cell that has a functional Fanconi anemia pathway with a test compound and a genotoxic anti-neoplastic agent, measuring the sensitivity of the test cell to the genotoxic anti-neoplastic agent, and comparing the sensitivity of the test cell to the agent to that of a control cell, wherein the control cell is isogenic to the test cell but has a mutant FANCI gene, and if the sensitivity of the test cell is greater than the sensitivity of the control cell, the test compound is identified as an inhibitor of a non-Fanconi anemia DNA repair pathway.

In one embodiment, the sensitivity of the test cell and the control cell to the anti-neoplastic agent is determined by measuring cell survival at one or more concentrations of the anti-neoplastic agent. In another embodiment, the test compound does not inhibit the Fanconi anemia pathway. In an additional embodiment, the mutant FANCI gene comprises a coding change that encodes a change in the FANCI polypeptide at K523, K1269, R1285, S730, T952, S1121, or P55. In another embodiment, the test cell and the control cell are human cells.

In another aspect, the invention provides a method of screening for a cancer therapeutic involving providing one or more cells containing a FANCI gene having one or more cancer associated defects, growing the cells in the presence of a potential cancer therapeutic, and determining the rate of growth of the cells in the presence of the potential cancer therapeutic relative to the rate of growth of equivalent cells grown in the absence of said potential cancer therapeutic, wherein a reduced rate of growth of the cells in the presence of the potential cancer therapeutic, relative to the rate of growth of equivalent cells grown in the absence of the potential cancer therapeutic, indicates that the potential cancer therapeutic is a cancer therapeutic.

In one embodiment, the FANCI gene having one or more cancer associated defects comprises a coding change that encodes a change in the FANCI polypeptide at K523, K1269, R1285, S730, T952, S1121, or P55. In another embodiment, the cells are human cells. In a related embodiment, the cells are BD0952 cells.

In an additional aspect, the invention provides a method of screening for a chemosensitizing agent involving providing a potential inhibitor of FANCI, providing a tumor cell line that is resistant to one or more anti-neoplastic agents, contacting the tumor cell line and the potential inhibitor of FANCI with the one or more anti-neoplastic agents, and measuring the growth rate of the tumor cell line in the presence of the inhibitor of FANCI and the anti-neoplastic agent, wherein a reduced growth rate of the tumor cell line, relative to cells of the tumor cell line in the presence of the anti-neoplastic agent and the absence of said inhibitor of FANCI, is indicative that the potential inhibitor is a chemosensitizing agent.

In a further aspect, the invention provides a method of sensitizing a subject to treatment with a genotoxic anti-neoplastic agent involving administering an inhibitor of FANCI to a subject who is receiving a genotoxic anti-neoplastic agent but is resistant to the agent.

In one embodiment, the inhibitor of FANCI is an antibody or antigen binding fragment thereof specific for FANCI or an anti-FANCI RNA interference agent. In another embodiment, the anti-FANCI RNA interference agent targets SEQ ID NO: 22, SEQ ID NO: 23, or SEQ ID NO: 24 in FANCI.

In another aspect, the invention provides a method of sensitizing a subject to treatment with a genotoxic anti-neoplastic agent involving administering an inhibitor of FANCI to a subject who is receiving treatment with a genotoxic anti-neoplastic agent but is resistant to the agent, and administering an inhibitor of a non-Fanconi anemia DNA repair pathway to the subject.

In one embodiment, the inhibitor of a non-Fanconi anemia DNA damage repair pathway is a PARP inhibitors, a DNA-PK inhibitor, an mTOR inhibitor, an ERCC1 inhibitor, an ERCC3 inhibitor, an ERCC6 inhibitor, an ATM inhibitor, an XRCC4 inhibitor, a Ku80 inhibitor, a Ku70 inhibitor, an XPA inhibitor, a CHK1 inhibitor, or a CHK2 inhibitor. In another embodiment, the genotoxic anti-neoplastic agent is admistered simultaneously with the inhibitor of FANCI and the inhibitor of a non-Fanconi anemia DNA repair pathway.

In an additional aspect, the invention provides a method of predicting the efficacy of a therapeutic agent in a cancer patient involving providing a tissue sample from the cancer patient who is being treated with the therapeutic agent, inducing DNA damage in the cells of the tissue sample, and detecting the presence of ubiquitinated FANCI protein in the cells, wherein presence of ubiquitinated FANCI is indicative of a reduced efficacy of the therapeutic agent in the cancer patient.

In a further embodiment, the invention provides a kit for determining whether a subject has cancer or is at increased risk of cancer, comprising an antibody or antigen binding fragment thereof specific for FANCI, packaging materials therefor, and instructions for performing a method of diagnosing or determining if a subject has cancer or is at increased risk of cancer. In another embodiment, the invention provides a kit for determining whether a subject with a neoplastic disorder will respond to a genotoxic anti-neoplastic agent, comprising an antibody or antigen binding fragment thereof specific for FANCI, packaging materials therefor, and instructions for performing a method of determining whether a subject with a neoplastic disorder will respond to a genotoxic anti-neoplastic agent. In an additional embodiment, the invention provides a kit for identifying an inhibitor of the Fanconi anemia pathway of an inhibitor of a non-Fanconi anemia pathway, comprising a test cell and a control cell for performance of screening methods as described in the methods of the invention, and packaging materials therefor.

In another aspect, the invention provides an isolated nucleotide or polypeptide sequence comprising the mutant FANCI nucleotide sequence of BD0952 cells.

In an additional aspect, the invention provides an isolated polypeptide sequence comprising GST fused to the N-terminal 200 amino acid residues of the FANCI polypeptide.

In a further aspect, the invention provides an anti-FANCI siRNA targeted to SEQ ID NO: 22, SEQ ID NO: 23, or SEQ ID NO: 24 of FANCI.

### BRIEF DESCRIPTION OF THE FIGURES

FIGS. 1A-1D show the experimental results used to identify KIAA1794 as the FANCI protein.

FIGS. 2A-2C show the identification of evolutionarily conserved regions of KIAA1794/FANCI.

FIGS. 3A-3F demonstrate checkpoint and repair defects in cells with reduced levels of FANCI.

FIGS. 4A-4D show that FANCI was identified to localize and interact with FANCD2.

FIGS. 5A-5I show FANCI ubiquitination and its dependence on the Fanconi anemia (FA) pathway.

FIGS. 6A-6F show complementation of BD0952 (FA-1) cells with the KIAA1794/FANCI gene.

FIGS. 7A and 7B demonstrate the localization of mutant FANCI alleles.

FIG. 8 shows the result of a MCA assay after ATM and ATR knockdown.

FIG. 9 shows cross-species conservation of FANCI sequence.

FIG. 10 shows conservation of FANCI and FANCD2 sequences.

FIGS. 11A-11E show that FANCI was identified to co-localize and interact with FANCD2.

FIGS. 12A-12C show FANCI ubiquitination.

FIG. 13 shows the localization of WT, P55L, R1285Q, and P55L, R1285Q mutant proteins in BD0952 (FA-I) cells.

### DETAILED DESCRIPTION OF THE INVENTION

The present invention, at least in part, is based on a series of discoveries showing the critical role played by the FA pathway in the sensitivity of cancers to anti-neoplastic agents. An additional role for DNA damage signaling in the FA pathway was discovered, and through a proteomic screen for substrates for the ATM and ATR kinases (Matsuoka et al., submitted) combined with a DNA damage sensitivity screen, the FANCI gene was identified. FANCI was identified a FANCD2 paralog, and was also shown to be monoubiquitinated on a lysine critical for its function. Accordingly, the present invention discloses that the FANCI protein is likely the second critical FA ligase substrate; and the FANCI polypeptide was shown to bind FANCD2 to form the ID complex that loads onto chromatin in response to DNA damage.

The role of other FA pathway components in modulating the sensitivity of neoplastic disorders and/or cancer cells to anti-neoplastic agents has been demonstrated using cell lines deficient in FA pathway components, and using inhibitors of the FA pathway. As a newly-identified component of the FA pathway that closely interacts with FANC D2, FANCI provides an attractive prognostic and diagnostic disease marker, genetic marker, and therapeutic target for use in screening methods to identify compounds capable of modulating FANCI activity and/or levels. Therefore, in one embodiment, a method for diagnosing or determining if a subject has cancer or is at increased risk of cancer is provided. One such method comprises monitoring the ubiquitination state and/or localization of FANCI to FANCI-comprising foci in an assessment of FANCI activity. Other aspects of the invention provide methods for predicting whether a subject with a neoplastic disorder and/or a tumor will respond to a genotoxic anti-neoplastic agent, involving assessment of the activity and/or polypeptide or nucleic acid sequence of FANCI in the subject. In certain embodiments, the method involves administering an effective dose of a FANCI inhibitor in combination with a genotoxic anti-neoplastic agent. Another method comprises administering an effective dose of a FANCI inhibitor in combination with an inhibitor of a non-FA DNA damage repair pathway.

Also provided are methods of identifying agents which modulate FANCI activity. Such methods are useful in identifying inhibitors of FANCI. Inhibitors thus identified are potentially useful as chemosensitizing and/or radiosensitizing agents. Also provided in the present invention are methods for identifying a non-FA DNA damage repair pathway inhibitor to be used in combination with the FANCI inhibitor. The combination of the inhibitors may be useful to administer to patients receiving anti-neoplastic agents.

### I. DEFINITIONS

As used herein, the terms “neoplasm”, “neoplastic disorder”, “neoplasia” “cancer,” “tumor” and “proliferative disorder” refer to cells having the capacity for autonomous growth, i.e., an abnormal state or condition characterized by rapidly proliferating cell growth which generally forms a distinct mass that show partial or total lack of structural organization and functional coordination with normal tissue. The terms are meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (relates to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures (including external epithelia (e.g., skin and linings of the gastrointestinal tract, lungs, and cervix), and internal epithelia that line various glands (e.g., breast, pancreas, thyroid). Examples of neoplasms that are particularly susceptible to treatment by the methods of the invention include leukemia, and hepatocellular cancers, sarcoma, vascular endothelial cancers, breast cancers, central nervous system cancers (e.g. astrocytoma, gliosarcoma, neuroblastoma, oligodendroglioma and glioblastoma), prostate cancers, lung and bronchus cancers, larynx cancers, esophagus cancers, colon cancers, colorectal cancers, gastro-intestinal cancers, melanomas, ovarian and endometrial cancer, renal and bladder cancer, liver cancer, endocrine cancer (e.g. thyroid), and pancreatic cancer.

A “genotoxic agent” or “genotoxin” refers to any chemical compound or treatment method that induces DNA damage when applied to a cell. Such agents can be chemical or radioactive. A genotoxic agent is one for which a primary biological activity of the chemical (or a metabolite) is alteration of the information encoded in the DNA. Genotoxic agents can vary in their mechanism of action, and can include: alkylating agents such as ethylmethane sulfonate (EMS), nitrosoguanine and vinyl chloride; bulky addition products such as benzo(a)pyrene and aflatoxin B1; reactive oxygen species such as superoxide, hydroxyl radical; base analogs such as 5-bromouracil; intercalating agents such as acridine orange and ethidium bromide.

A “genotoxic anti-neoplastic agent”, as used herein, is a genotoxic agent used for chemotherapy, for example, to treat cancer. In particular, “genotoxic anti-neoplastic agents” encompass agents, both chemical or otherwise, which cause damage to DNA. These agents include DNA alkylating agents, intercalating agents, and the like. Non-limiting examples of “genotoxic anti-neoplastic agents” include 1,3-Bis(2-Chloroethyl)-1-NitrosoUrea (BCNU), Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolomide, and Topotecan. “Genotoxic anti-neoplastic agents” also include radiation, in particular the types used in radiation therapy for the treatment of cancer, in a dosages sufficient to cause damage to DNA in a subject.

“DNA damage”, as used herein, refers to chemical and/or physical modification of the DNA in a cell, including methylation, alkylation double-stranded breaks, cross-linking, thymidine dimers caused by ultraviolet light, and oxidative lesions formed by oxygen radical binding to DNA bases.

As used herein, a “chemosensitizer” and “chemosensitizing agent” refer to a compound which, when administered in a therapeutically effective amount in a subject, increases the sensitivity to chemotherapy compounds, and/or increases the therapeutic efficacy of the compounds, for example, in the treatment of a disease, such as neoplastic diseases, benign and malignant tumors, and cancerous cells. An increase in sensitivity to chemotherapy compounds, including genotoxic anti-neoplastic agents, can be measured, for example, by measuring the decrease in LD₅₀ of a cell towards a compound in the presence of the chemosensitizer.

Similarly, a “radiosensitizer” and “radiosensitizing agent”, as used herein, refer to a compound which, when administered in a therapeutically effective amount in a subject, increases the sensitivity to radiation therapy (treatment by electromagnetic radiation), and/or increases the therapeutic efficacy of radiation therapy, for example, in the treatment of a disease, such as neoplastic diseases, benign and malignant tumors and cancer cells. Also contemplated are electromagnetic radiation treatment of other diseases not listed herein.

“Cancer-Associated Coding Change” refers to any sequence change in the amino acid sequence of a protein encoded by a FANC/BRCA gene, as defined herein, harbors a defect, as defined herein, that can cause or is associated with a cancer in a patient.

Similarly, “Fanconi Anemia-Associated Coding Change” refers to any sequence change in the amino acid sequence of a protein encoded by a Fanconi anemia pathway gene, as defined herein, harbors a defect, as defined herein, that can cause or is associated with Fanconi anemia in a patient.

As used herein, “testing a FANCI gene for the presence of a cancer-associated defect” refers to the method of determining if a protein encoded by a FANCI gene harbors a defect, as defined herein, that can cause or is associated with a cancer in a subject.

As used herein, the term “defect” refers to any alteration of a gene or protein within the Fanconi Anemia BRCA pathway, and/or proteins, with respect to any unaltered gene or protein within the Fanconi Anemia/BRCA pathway.

“Alteration” of a gene includes, but is not limited to: a) alteration of the DNA sequence itself, i.e., DNA mutations, deletions, insertions, substitutions; b) DNA modifications affecting the regulation of gene expression such as regulatory region mutations, modification in associated chromatin, modications of intron sequences affecting mRNA splicing, modification affecting the methylation/demethylation state of the gene sequence; c) mRNA medications affecting protein translation or mRNA transport or mRNA splicing.

“Alteration” of a protein includes, but is not limited to, amino acid deletions, insertions, substitutions; modification affecting protein phosphorylation or glycosylation; modifications affecting protein transport or localization; modifications affecting the ability to form protein complexes with one or more associated proteins or changes in the amino acid sequence caused by changes in the DNA sequence encoding the amino acid.

As used herein, the term “increased risk” or “elevated risk” refers to the greater incidence of cancer in those patients having altered Fanconi Anemia/BRCA genes or proteins as compared to those patients without alterations in the Fanconi Anemia/BRCA pathway genes or proteins. “Increased risk” also refers to patients who are already diagnosed with cancer and may have an increased incidence of a different cancer form. According to the invention, “increased risk” of cancer refers to cancer-associated defects in a Fanconi Anemia/BRCA pathway gene that contributes to a 50%, preferably 90%, more preferably 99% or more increase in the probability of acquiring cancer relative to patients who do not have a cancer-associated defect in a Fanconi Anemia/BRCA pathway gene.

As used herein, the term “inducing DNA damage” refers to both chemical and physical methods of damaging DNA. Chemicals that damage DNA include, but are not limited to, acids/bases and various mutagens, such as ethidium bromide, acridine orange, as well as free radicals. Physical methods include, but are not limited to, ionizing radiation, such as X rays and gamma rays, and ultraviolet (UV) radiation. Both methods of “inducing DNA damage” can result in DNA mutations that typically include, but are not limited to, single-strand breaks, double-strand breaks, alterations of bases, insertions, deletions or the cross-linking of DNA strands.

By “sample” or “biological sample” is meant any cell or tissue, or cell or tissue-containing composition or isolate derived from the subject. The sample may be derived from heart, brain, placenta, liver, skeletal muscle, kidney, pancreas, spleen, thymus, prostate, testis, uterus, small intestine, or colon. Another type of biological sample may be a preparation containing white blood cells, e.g., peripheral blood, sputum, saliva, urine, etc., for use in detecting the presence or absence of DNA damage in a subject that has been exposed to a genotoxic agent, such as radiation, chemicals, etc.

As used herein, the term “tissue biopsy” refers to a biological material, which is isolated from a patient. The term “tissue”, as used herein, is an aggregate of cells that perform a particular function in an organism and encompasses cell lines and other sources of cellular material including, but not limited to, a biological fluid for example, blood, plasma, sputum, urine, cerebrospinal fluid, lavages, and leukophoresis samples.

As used herein, “degree of ubiquitination” of the FANCI polypeptide refers generally to the level of activation of the FA pathway, as measured by the degree of monoubiquitination of the FANCI polypeptide within a subject or biological sample therefrom. As used herein, the “degree of ubiquitination” of the FANCI polypeptide can encompass the proportion of total FANCI polypeptide within a sample that is monoubiquitinated, and can be expressed on a fractional or percentage basis. As used herein, the “degree of ubiquitination” of the FANCI polypeptide can also be measured using any substitute methods of detecting activation of the FA pathway, including the degree of foci formation.

As used herein, “degree of foci formation” refers to the total number or the rate of formation of FANCI-containing foci in a sample. FANCI-containing foci are nuclear protein complexes formed in response to the activation of the FA pathway, for example by exposure to a genotoxic agent. FANCI-containing foci can be detected, for example, by immunofluorescence microscopy using a labeled antibody directed against the FANCI polypeptide, as further described herein. In certain cases, FANCI-containing foci can also be detected in cells expressing a functional fusion protein comprising GFP and the FANCI polypeptide. In these cells, FANCI-containing foci can be detected using fluorescence microscopy without the use of anti-FANCI antibodies. The degree of foci formation can be normalized from one sample to another, for example, to total number of cells, total number of intact nuclei, total sample volume, or total sample mass.

By “difference in foci formation” is meant a difference, whether higher or lower, in the number, size or persistence of FANCI-containing foci, when comparing a test sample with either a control sample or reference sample. A difference includes an increase or decrease that is 2-fold or more, or less, for example 5, 10, 20, 100, 1000-fold or more as compared to a control or reference sample. A difference also includes an increase or decrease that is 5% more or less, for example, 10%, 20%, 30%, 50%, 75%, 100%, as compared to a control or reference sample.

“Modulate” formation of FANCI-containing foci, as used herein, refers to a change or an alteration in the formation of FANCI-containing foci in a biological sample. Modulation may be an increase or a decrease in foci number, size or persistence within a biological sample, and includes an increase or decrease that is 2-fold or more, or less, for example 5, 10, 20, 100, 1000-fold or more as compared to a control or reference sample. Modulation may also be an increase or decrease that is 5% more or less, for example, 10%, 20%, 30%, 50%, 75%, 100%, as compared to a control or reference sample.

As used herein, exposure to a “low level” of a genotoxic anti-neoplastic agent refers to exposure to a dose of a particular genotoxic anti-neoplastic agent which results in no more than 20% of the maximal number of FANCI-containing foci in biological samples. Because of the multitude of genotoxic anti-neoplastic agents to which a sample may be exposed, as well as the varying sensitivities of different samples to such genotoxic anti-neoplastic agents, it is preferable to express the dosage relative to the formation of FANCI-containing foci, rather than in the absolute dose of a particular genotoxic anti-neoplastic agent.

The term “modulator” refers to a chemical compound (naturally occurring or non-naturally occurring), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Modulators are evaluated for potential activity as inhibitors or activators (directly or indirectly) of a biological process or processes (e.g., agonist, partial antagonist, partial agonist, antagonist, anti-neoplastic agents, cytotoxic agents, inhibitors of neoplastic transformation or cell proliferation, cell proliferation-promoting agents, and the like) by inclusion in screening assays described herein. The activities (or activity) of a modulator may be known, unknown or partially-known. Such modulators can be screened using the methods described herein.

The term “candidate modulator” refers to a compound to be tested by one or more screening method(s) of the invention as a putative modulator. Usually, various predetermined concentrations are used for screening such as 0.0 μM, 0.1 μM, 1.011M, and 10.0 μM, as described more fully below. Test compound controls can include the measurement of a signal in the absence of the test compound or comparison to a compound known to modulate the target.

As used herein, an “FA pathway inhibitor” and “inhibitor of the FA pathway” refer to a chemical compound (naturally occurring or non-naturally occurring), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. An “FA pathway inhibitor” and “inhibitor of the FA pathway” refer broadly to compounds which inhibit the ability of the FA pathway to repair DNA damage. Inhibition of the FA pathway by an “FA pathway inhibitor” or an “inhibitor of the FA pathway” can be assessed using the techniques described herein, including without limitation, the detection of FANCI-containing foci and detection of monoubiquitination of the FANCI polypeptides. As will be appreciated by one skilled in the art, the method contemplates any other method currently known or known in the future, for the detection of the inhibition of the FA pathway. Inhibition may be a decrease in number, size or persistence of FANCI-containing foci, and includes a decrease that is 2-fold or more, for example, 2, 5, 10, 20, 100, 1000-fold or more as compared to a control or reference. Inhibition may also be an decrease of 5% or more, for example 5%, 10%, 20%, 30%, 50%, 75%, or up to 100%, as compared to a control or reference. In addition, as used herein, an “FA pathway inhibitor” and “inhibitor of the FA pathway” encompass the pharmaceutically acceptable salts, solvates, esters, derivatives or prodrugs.

A “non-FA DNA damage repair pathway”, as used herein, refers to any of the DNA damage repair pathways selected from the group consisting of the direct reversal, non-homologous end joining (NHEJ), base excision repair (BER), nucleotide excision repair (NER), and mismatch repair (MR) pathways.

As used herein, the term “amplifying”, when applied to a nucleic acid sequence, refers to a process whereby one or more copies of a particular nucleic acid sequence is generated from a template nucleic acid, preferably by the method of polymerase chain reaction (Mullis and Faloona, 1987, Methods Enzymol., 155:335). “Polymerase chain reaction” or “PCR” refers to an in vitro method for amplifying a specific nucleic acid template sequence. The PCR reaction involves a repetitive series of temperature cycles and is typically performed in a volume of 50-100.mu.l. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers” or “a set of PCR primers” can comprise two, three, four or more primers.

Other methods of amplification include, but are not limited to, ligase chain reaction (LCR), polynucleotide-specific base amplification (NSBA), or any other method known in the art.

As used herein, the term “polynucleotide primer” refers to a DNA or RNA molecule capable of hybridizing to a nucleic acid template and acting as a substrate for enzymatic synthesis under conditions in which synthesis of a primer extension product which is complementary to a nucleic acid template is catalyzed to produce a primer extension product which is complementary to the target nucleic acid template. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer is preferably single-stranded for maximum efficiency in amplification. “Primers” useful in the present invention are generally between about 10 and 35 nucleotides in length, preferably between about 15 and 30 nucleotides in length, and most preferably between about 18 and 25 nucleotides in length.

As used herein, the term “antibody” refers to an immunoglobulin having the capacity to specifically bind a given antigen. The term “antibody” as used herein is intended to include whole antibodies of any isotype (IgG, IgA, IgM, IgE, etc), and fragments thereof which are also specifically reactive with a vertebrate, e.g., mammalian, protein. Antibodies can be fragmented using conventional techniques and the fragments screened for utility in the same manner as whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab, Fv, and single chain antibodies (scFv) containing a V[L] and/or V[H] domain joined by a peptide linker. The scFv's may be covalently or non-covalently linked to form antibodies having two or more binding sites. Antibodies may be labeled with detectable moieties by one of skill in the art. In some embodiments, the antibody that binds to an entity one wishes to measure (the primary antibody) is not labeled, but is instead detected by binding of a labeled secondary antibody that specifically binds to the primary antibody.

A patient is “treated” according to the invention if one or preferably more symptoms of cancer as described herein are eliminated or reduced in severity, or prevented from progressing or developing further.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions.

As used herein, the term “cancer therapeutic” refers to a compound that prevents the onset or progression of cancer or prevents cancer metastasis or reduces, delays, or eliminates the symptoms of cancer.

“Ubiquitination” is defined as the covalent linkage of ubiquitin to a protein by a E3 mono-ubiquitin ligase.

As used herein, the term “cisplatin” refers to an agent with the following chemical structure:

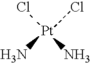

Cisplatin, also called cis-diamminedichloroplatinum(II), is one of the most frequently used anticancer drugs. It is an effective component of several different combination drug protocols used to treat a variety of solid tumors. These drugs are used in the treatment of testicular cancer (with bleomycin and vinblastine), bladder cancer, head and neck cancer (with bleomycin and fluorouracil), ovarian cancer (with cyclophosphamide or doxorubicin) and lung cancer (with etoposide). Cisplatin has been found to be the most active single agent against most of these tumors. Cisplatin is commercially available as ‘Platinol’ from Bristol Myers Squibb Co. Cisplatin is one of a number of platinum coordination complexes with antitumor activity. The platinum compounds are DNA cross-linking agents similar to but not identical to the alkylating agents. The platinum compounds exchange chloride ions for nucleophilic groups of various kinds. Both the cis and trans isomers do this but the trans isomer is known to be bioligically inactive for reasons not completely understood. To possess antitumor activity a platinum compound must have two relatively labile cis-oriented leaving groups. The principal sites of reaction are the N7 atoms of guanine and adenine. The main interaction is formation of intrastrand cross links between the drug and neighboring guanines. Intrastrand cross linking has been shown to correlate with clinical response to cisplatin therapy. DNA/protein cross linking also occurs but this does not correlate with cytotoxicity. Cross-resistance between the two groups of drugs is usually not seen indicating that the mechanisms of action are not identical. The types of cross linking with DNA may differ between the platinum compounds and the typical alkylating agents.

As used herein, “resistance to one or more anti-neoplastic agents” refers the ability of cancer cells to develop resistance to anticancer drugs. Mechanisms of drug resistance include decreased intracellular drug levels caused by an increased drug efflux or decreased inward transport, increased drug inactivation, decreased conversion of drug to an active form, altered amount of target enzyme or receptor (gene amplification), decreased affinity of target enzyme or receptor for drug, enhanced repair of the drug-induced defect, decreased activity of an enzyme required for the killing effect (topoisomerase II). In a preferred embodiment of the invention, drug resistance refers to the enhanced repair of DNA damage induced by one or more anti-neoplastic agents. In another preferred embodiment of the invention, the enhanced repair of DNA damage induced by one or more anti-neoplastic agents is due to a constitutively active Fanconi Anemia/BRCA DNA repair pathway.

As used herein, the term “anti-neoplastic agent” refers to a compound that is used to treat cancer. According to the invention, an “anti-neoplastic agent” encompasses chemotherapy compounds as well as other anti-cancer agents known in the art. In a preferred embodiment, the “anti-neoplastic agent” is cisplatin. Anti-neoplastic agents according to the invention also include cancer therapy protocols using chemotherapy compounds in conjunction with radiation therapy and/or surgery. Radiation therapy relies on the local destruction of cancer cells through ionizing radiation that disrupts cellular DNA. Radiation therapy can be externally or internally originated, high or low dose, and delivered with computer-assisted accuracy to the site of the tumor. Brachytherapy, or interstitial radiation therapy, places the source of radiation directly into the tumor as implanted “seeds.”

As used herein, the term “a reduced growth rate” refers to a decrease of 50%, preferably 90%, more preferably 99% and most preferably 100% in the rate of cellular proliferation of a tumor cell line that is being treated with a potential inhibitor of the Fanconi Anemia/BRCA pathway and one or more chemotherapy compounds relative to cells of a tumor cell line that is not being treated with a potential inhibitor of the Fanconi Anemia/BRCA pathway and one or more chemotherapy compounds.

The pharmaceutical compositions of the present invention can be administered using any amount and any route of administration effective for increasing the therapeutic efficacy of drugs. As used herein, “therapeutically effective amount,” when used in combination with a chemosensitizer or radiosensitizer, refers to a sufficient amount of the chemosensitizing agent to provide the desired effect against target cells or tissues. The exact amount required will vary from subject to subject, depending on the species, age, and general condition of the subject; the particular chemosensitizing agent; its mode of administration; and the like.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “therapeutically effective dose” refers to that amount of protein or its antibodies, antagonists, or inhibitors which prevent or ameliorate the symptoms or conditions, for example, a neoplastic disorder. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. Pharmaceutical compositions which exhibit large therapeutic indices are preferred. The data obtained from cell culture assays and animals studies is used in formulating a range of dosage for human use. The dosage of such compounds lies preferably within a range of circulating concentrations that include the ED50 with little or no toxicity. The dosage varies within this range depending upon the dosage from employed, sensitivity of the patient, and the route of administration.

The exact dosage is chosen by the individual physician or veterinarian in view of the patient to be treated. Dosage and administration are adjusted to provide sufficient levels of the active moiety or to maintain the desired effect. Additional factors which may be taken into account include the severity of the disease state; age, weight and gender of the subject; diet, time and frequency of administration, drug combination(s), reaction sensitivities, and tolerance/response to therapy. Long acting pharmaceutical compositions might be administered every 3 to 4 days, every week, or once every two weeks depending on a half-life and clearance rate of the particular formulation.

The term “pharmaceutically acceptable salt” refers to both acid addition salts and base addition salts. The nature of the salt is not critical, provided that it is pharmaceutically acceptable. Exemplary acid addition salts include, without limitation, hydrochloric, hydrobromic, hydroiodic, nitric, carbonic, sulfuric, phosphoric, formic, acetic, citric, tartaric, succinic, oxalic, malic, glutamic, propionic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric, galacturonic acid and the like. Suitable pharmaceutically acceptable base addition salts include, without limitation, metallic salts made from aluminum, calcium, lithium, magnesium, potassium, sodium and zinc or organic salts made from N,N′-dibenzylethylenediamine, chloroprocaine, choline, diethanolamine, ethylenediamine, N-methylglucamine, lysine, procaine and the like. Additional examples of pharmaceutically acceptable salts are listed in Journal of Pharmaceutical Sciences (1977) 66:2. All of these salts may be prepared by conventional means from a modulator of FANCI-containing foci by treating the compound with the appropriate acid or base.

The term “subject” is intended to include living organisms in which neoplasia can occur. Examples of subjects include, but are not limited to, humans, monkeys, cows, sheep, goats, dogs, cats, mice, rats, and transgenic species thereof.

The term “RNA interference” or “RNAi” (also referred to in the art as “gene silencing” and/or “target silencing”, e.g., “target mRNA silencing”), as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNAi agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “RNAi agent”, as used herein, refers to an RNA (or analog thereof), having sufficient sequence complementarity to a target RNA (i.e., the RNA being degraded) to direct RNAi. A RNAi agent having a “sequence sufficiently complementary to a target RNA sequence to direct RNAi” means that the RNAi agent has a sequence sufficient to trigger the destruction of the target RNA by the RNAi machinery (e.g., the RISC) or process. A RNAi agent having a “sequence sufficiently complementary to a target RNA sequence to direct RNAi” is also intended to mean that the RNAi agent has a sequence sufficient to trigger the translational inhibition of the target RNA by the RNAi machinery or process. A RNAi agent having a “sequence sufficiently complementary to a target RNA encoded by the target DNA sequence such that the target DNA sequence is chromatically silenced” means that the RNAi agent has a sequence sufficient to induce transcriptional gene silencing, e.g., to down-modulate gene expression at or near the target DNA sequence, e.g., by inducing chromatin structural changes at or near the target DNA sequence.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. Preferably, an siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs).

It will be appreciated by the skilled artisan that even a single substitution in a nucleic acid or gene sequence (e.g., a base substitution that encodes an amino acid change in the corresponding amino acid sequence) can dramatically affect the activity of an encoded polypeptide or protein as compared to the corresponding wild-type polypeptide or protein. A mutant nucleic acid or mutant gene (e.g., encoding a mutant polypeptide or protein), as defined herein, is readily distinguishable from a nucleic acid or gene encoding a protein homologue or paralog in that a mutant nucleic acid or mutant gene encodes a protein or polypeptide having an altered activity, optionally observable as a different or distinct phenotype in a microorganism, cell or organism expressing said mutant gene or nucleic acid or producing said mutant protein or polypeptide (i.e., a mutant cell line) as compared to a corresponding microorganism, cell or organism expressing the wild-type gene or nucleic acid or producing said mutant protein or polypeptide. By contrast, a protein homolog or paralog has an identical or substantially similar activity, optionally phenotypically indiscemable when produced in a microorganism, cell or organism, as compared to a corresponding microorganism, cell or organism expressing the wild-type gene or nucleic acid. Accordingly it is not, for example, the degree of sequence identity between nucleic acid molecules, genes, protein or polypeptides that serves to distinguish between homologues (or paralogs) and mutants, rather it is the activity of the encoded protein or polypeptide that distinguishes between homologues and mutants: homologues and/or paralogs having, for example, low (e.g., 30-50% sequence identity) sequence identity yet having substantially equivalent functional activities, and mutants, for example sharing 99% sequence identity yet having dramatically different or altered functional activities.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes.

Various aspects of the invention are described in further detail in the following subsections.

### II. FANCI FOCI

The cellular response to DNA damage is a complex interacting network of pathways that mediate cell cycle checkpoints, DNA repair, and apoptosis. A model lesion for the investigation of these pathways has been DNA double-strand breaks, which rapidly induce cell cycle checkpoints and are repaired by a number of different pathways. In mammalian cells, both homologous recombination and nonhomologous recombination pathways are utilized. Extensive studies in mammalian cells have shown that complexes of DNA repair and cell cycle checkpoint proteins rapidly localize to sites of double-strand breaks induced by ionizing radiation. These proteins create foci that can be detected by immunofluorescent analyses.

The Fanconi anemia complementation group I (FANCI), like its paralog, FANC D2, is a component of a protein complex involved in chromosome stability and repair. Fanconi anemia (FA) is a hereditary disorder characterized, in part, by a deficient DNA-repair mechanism that increases a person's risk for a variety of cancers. In response to DNA damage, the FA complex activates FANC D2, which then associates with Breast Cancer, Type 1 polypeptide (BRCA1). Activation of FANC D2 occurs by phosphorylation of a serine 222 residue by the Ataxia-Telangiectasia Mutated (ATM) kinase. In addition, activation via the FA pathway occurs via monoubiquitination of FANC D2 at lysine 561. In its unmodified form, FANC D2 is diffusely located throughout the nucleus. When ubiquitinated, FANC D2 forms dots, or foci, in the nucleus. The ubiquitination of FANC D2 and subsequent formation of nuclear foci occurs in response to DNA damage. By coimmunoprecipitation, Nakanishi et al. found constitutive interaction between FANC D2 and Nijmegen Breakage Syndrome 1 (NBS1), providing evidence that these proteins interact in two distinct assemblies to mediate S-phase checkpoint and resistance to mitomycin C-induced chromosome damage (Nakashini et al., (2002) Nat Cell Biol. 4:913-20). The instant identification of FANCI as a monoubiquitinated phosphoprotein that is phosphorylated by ATM and co-localizes with FANC D2 in foci indicates that FANCI, like FANC D2, also interacts with BRCA1 and constitutively interacts with NBS1, to mediate S-phase checkpoint and resistance to MMC-induced chromosome damage.

At least two types of ionizing radiation-induced foci have been observed: one containing the Rad51, BRCA1 and BRCA2 proteins, and another containing the Mre11-Rad50-NBS1 complex. Rad51 foci, which contain the tumor suppressor proteins BRCA1 and BRCA2, also appear during S phase in the absence of exogenous induction of DNA damage.

Mre11-Rad50-NBS1 foci can be detected as early as 10 min after irradiation and are clearly present at sites of DNA breaks, while DNA repair is ongoing. These foci also colocalize with the BRCA1 protein, which has been shown to be required for their formation, possibly through its physical interaction with human Rad50 (hRad50). In addition, coimmunoprecipitation experiments performed with BRCA1 have indicated the presence of a large number of additional proteins in this complex (referred to as the BRCA1-associated surveillance complex). These include the mismatch repair proteins Msh2, Msh6, and Mlh1, the checkpoint kinase ATM, the product of the Bloom's syndrome gene BLM, and replication factor C. BRCA1, NBS1, and hMre11 have all been shown to be substrates of the ATM kinase and to become phosphorylated in response to the presence of DNA breaks.

The present invention is related to the discovery that cells exposed to genotoxic anti-neoplastic agents form FANCI-containing foci that correspond to the FANC D2-containing foci previously identified and described, e.g., in U.S. application Ser. No. 11/441,289, U.S. App. No. 60/684,136, U.S. application Ser. No. 11/046,346, and U.S. App. No. 60/540,380, the contents of which are incorporated in their entirety herein by reference. Multiple DNA damage response proteins have now been identified which form nuclear foci, also called IRIFs (Ionizing-Radiation Inducible foci) in response to DNA damage. Methods of detecting FANC D2-containing foci, as well as detecting and quantitating the relative amount of ubiquitinated FANC D2 polypeptide are described in U.S. application Ser. No. 10/165,099 and U.S. App. No. 60/540,380, the contents of which are incorporated in their entirety herein by reference.

### III. MEANS OF DETECTING FANCI ACTIVATION

### 1. Detection Using FANCI-Binding Ligands

As disclosed herein, FANCI can be readily detected using antibodies that specifically bind FANCI. Commercially available antibodies disclosed herein to specifically bind to FANCI include anti-KIAA1794 antibodies BL999 and BL1000 (Bethyl). Additional antibodies that specifically bind FANCI can be readily prepared by the methods described herein, including, e.g., monoclonal antibodies to FANCI.

The antibodies employed in the invention specifically bind to FANCI. As used herein in reference to antibody binding, FANCI includes the FANCI protein, and fragments thereof. Such fragments may be entire domains, and may also include contiguous and noncontiguous epitopes in any domain of the FANCI protein. Examples of antigens used to raise antibodies specific for FANCI include, but are not limited to the amino acid sequences described in Example 1.

Once antibodies to FANCI are generated, binding of the antibodies to FANCI may be assayed using standard techniques known in the art, such as ELISA, while the localization of FANCI within a cell may be assayed using the techniques disclosed in the Examples. Any other techniques of measuring such binding may alternatively be used.

This invention employs antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for FANCI or fragments thereof. The terms “specific” and “selective,” when used to describe binding of the antibodies of the invention, indicate that the variable regions of the antibodies of the invention recognize and bind FANCI polypeptides. It will be understood that specific antibodies of the invention may also interact with other proteins (for example, S. aureus protein A or other antibodies in ELISA techniques) through interactions with sequences outside the variable regions of the antibodies, and, in particular, in the constant regions of the molecule.

Screening assays to determine binding specificity of an antibody of the invention (e.g., antibodies that specifically bind to a FANCI epitope) are well known and routinely practiced in the art. For a comprehensive discussion of such assays, see Harlow et al. (Eds.), Antibodies A Laboratory Manual; Cold Spring Harbor Laboratory; Cold Spring Harbor, N.Y. (1988), Chapter 6. Antibodies that recognize and bind fragments of FANCI protein are also included, provided that the antibodies are specific for FANCI polypeptides. Antibodies of the invention can be produced using any method well known and routinely practiced in the art.

It should be emphasized that antibodies that can be generated from other polypeptides that have previously been described in the literature and that are capable of fortuitously cross-reacting with FANCI (e.g., due to the fortuitous existence of a similar epitope in both polypeptides) are considered “cross-reactive” antibodies. Such cross-reactive antibodies are not antibodies that are “specific” for FANCI. The determination of whether an antibody specifically binds to an epitope of FANCI is made using any of several assays, such as western blotting assays, that are well known in the art. For identifying cells that express FANCI and also for inhibiting FANCI activity, antibodies that specifically bind to an epitope of the FANCI protein are particularly useful.

In certain embodiments, the invention employs polyclonal antibodies, wherein at least one of the antibodies is an antibody specific for FANCI. Antiserum isolated from an animal is an exemplary composition, as is a composition comprising an antibody fraction of an antiserum that has been resuspended in water or in another diluent, excipient, or carrier.

In other embodiments, the invention employs monoclonal antibodies. Monoclonal antibodies are highly specific, being directed against a single antigenic site. Further, in contrast to polyclonal preparations which typically include different antibodies directed against different epitopes, each monoclonal antibody is directed against a single determinant on the antigen. Monoclonal antibodies are useful to improve selectivity and specificity of diagnostic and analytical assay methods using antigen-antibody binding. Another advantage of monoclonal antibodies is that they can be synthesized by cultured cells such as hybridomas, uncontaminated by other immunoglobulins. Recombinant cells and hybridomas that produce such antibodies are also intended for use within certain aspects of the invention.

In still other related embodiments, the invention can employ an anti-idiotypic antibody specific for an antibody that is specific for FANCI. For a more detailed discussion of anti-idiotypic antibodies, see, e.g., U.S. Pat. Nos. 6,063,379 and 5,780,029.

It is well known that antibodies contain relatively small antigen binding domains that can be isolated chemically or by recombinant techniques. Such domains are useful FANCI binding molecules themselves, and also may be reintroduced into human antibodies, or fused to a chemotherapeutic or polypeptide. Thus, in still another embodiment, the invention employs a polypeptide comprising a fragment of a FANCI-specific antibody, wherein the fragment and associated molecule, if any, bind to FANCI. By way of non-limiting example, the invention can employ polypeptides that are single chain antibodies and CDR-grafted antibodies. For a more detailed discussion of CDR-grafted antibodies, see, e.g., U.S. Pat. No. 5,859,205 and discussion below.

In other embodiments, non-human antibodies may be humanized by any of the methods known in the art. Humanized antibodies are useful for in vivo therapeutic applications. In addition, recombinant “humanized” antibodies can be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences in which the regions responsible for specific antigen-binding have been replaced.

Various forms of antibodies may be produced using standard recombinant DNA techniques (Winter and Milstein, 1991, Nature 349:293-99). For example, the monoclonal antibodies of this invention can be generated by well known hybridoma technology. For instance, animals (e.g., mice, rats or rabbits) can be immunized with purified or crude FANCI preparations, cells transfected with cDNA constructs encoding FANCI, cells that constitutively express FANCI, and the like. In addition, the antigen can be delivered as purified protein, protein expressed on cells, protein fragment or peptide thereof, or as naked DNA or viral vectors encoding the protein, protein fragment, or peptide. Sera of the immunized animals are then tested for the presence of anti-FANCI antibodies. B cells are isolated from animals that test positive, and hybridomas are made with these B cells.

Antibodies secreted by the hybridomas are screened for their ability to bind specifically to FANCI (e.g., binding to FANCI-transfected cells and not to untransfected parent cells) and for any other desired features, e.g., having the desired CDR consensus sequences, inhibiting (or not in the case of nonblockers) the binding between FANCI and FANC D2 or inhibiting formation of FANCI-containing foci.

Hybridoma cells that test positive in the screening assays are cultured in a nutrient medium under conditions that allow the cells to secrete the monoclonal antibodies into the culture medium. The conditioned hybridoma culture supernatant is then collected and antibodies contained in the supernatant are purified. Alternatively, the desired antibody may be produced by injecting the hybridoma cells into the peritoneal cavity of an unimmunized animal (e.g., a mouse). The hybridoma cells proliferate in the peritoneal cavity, secreting the antibody which accumulates as ascites fluid. The antibody may then be harvested by withdrawing the ascites fluid from the peritoneal cavity with a syringe.

The monoclonal antibodies can also be generated by isolating the antibody-coding cDNAs from the desired hybridomas, transfecting mammalian host cells (e.g., CHO or NSO cells) with the cDNAs, culturing the transfected host cells, and recovering the antibody from the culture medium.

The monoclonal antibodies employed in this invention can also be generated by engineering a cognate hybridoma (e.g., murine, rat or rabbit) antibody. For instance, a cognate antibody can be altered by recombinant DNA technology such that part or all of the hinge and/or constant regions of the heavy and/or light chains are replaced with the corresponding components of an antibody from another species (e.g., human). Generally, the variable domains of the engineered antibody remain identical or substantially so to the variable domains of the cognate antibody. Such an engineered antibody is called a chimeric antibody and is less antigenic than the cognate antibody when administered to an individual of the species from which the hinge and/or constant region is derived (e.g., a human). Methods of making chimeric antibodies are well known in the art. Human constant regions include those derived from IgG1 and IgG4.

The monoclonal antibodies employed in this invention also include fully human antibodies. They may be prepared using in vitro-primed human splenocytes, as described by Boerner et al., 1991, J. Immunol. 147:86-95, or using phage-displayed antibody libraries, as described in, e.g., U.S. Pat. No. 6,300,064.

Alternatively, fully human antibodies may be prepared by repertoire cloning as described by Persson et al., 1991, Proc. Natl. Acad. Sci. USA 88:2432-36; and Huang and Stollar, 1991, J. Immunol. Methods 141:227-36. In addition, U.S. Pat. No. 5,798,230 describes preparation of human monoclonal antibodies from human B cells, wherein human antibody-producing B cells are immortalized by infection with an Epstein-Barr virus, or a derivative thereof, that expresses Epstein-Barr virus nuclear antigen 2 (EBNA2), a protein required for immortalization. The EBNA2 function is subsequently shut off, resulting in an increase in antibody production.

Some other methods for producing fully human antibodies involve the use of non-human animals that have inactivated endogenous Ig loci and are transgenic for un-rearranged human antibody heavy chain and light chain genes. Such transgenic animals can be immunized with FANCI and hybridomas made from B cells derived therefrom. These methods are described in, e.g., the various GenPharm/Medarex (Palo Alto, Calif.) publications/patents concerning transgenic mice containing human Ig miniloci (e.g., U.S. Pat. No. 5,789,650); the various Abgenix (Fremont, Calif.) publications/patents with respect to XENOMICE™ (e.g., U.S. Pat. Nos. 6,075,181, 6,150,584 and 6,162,963; Green et al., 1994, Nature Genetics 7:13-21; and Mendez et al., 1997, Nature Genetics 15:146-56); and the various Kirin (Japan) publications/patents concerning “transomic” mice (e.g., EP 843 961, and Tomizuka et al., 1997, Nature Genetics 16:1433-43). See also, e.g., U.S. Pat. No. 5,569,825, WO00076310, WO00058499 and WO00037504, incorporated by reference herein in their entireties.

The monoclonal antibodies employed in this invention also include humanized versions of cognate anti-FANCI antibodies derived from other species. A humanized antibody is an antibody produced by recombinant DNA technology, in which some or all of the amino acids of a human immunoglobulin light or heavy chain that are not required for antigen binding (e.g., the constant regions and the framework regions of the variable domains) are used to substitute for the corresponding amino acids from the light or heavy chain of the cognate, nonhuman antibody. By way of example, a humanized version of a murine antibody to a given antigen has on both of its heavy and light chains (1) constant regions of a human antibody; (2) framework regions from the variable domains of a human antibody; and (3) CDRs from the murine antibody. When necessary, one or more residues in the human framework regions can be changed to residues at the corresponding positions in the murine antibody so as to preserve the binding affinity of the humanized antibody to the antigen. This change is sometimes called “back mutation.” Humanized antibodies generally are less likely to elicit an immune response in humans as compared to chimeric human antibodies because the former contain considerably fewer non-human components.

The methods for making humanized antibodies are described in, e.g., Winter EP 239 400; Jones et al., 1986, Nature 321:522-25; Riechmann et al., 1988, Nature 332:323-27 (1988); Verhoeyen et al., 1988, Science 239:1534-36; Queen et al., 1989, Proc. Natl. Acad. Sci. USA 86:10029-33; U.S. Pat. No. 6,180,370; and Orlandi et al., 1989, Proc. Natl. Acad. Sci. USA 86:3833-37. See also, e.g., PCT patent application No. 94/04679. Primatized antibodies can be produced similarly using primate (e.g., rhesus, baboon and chimpanzee) antibody genes. Further changes can then be introduced into the antibody framework to modulate affinity or immunogenicity. See, e.g., U.S. Pat. Nos. 5,585,089, 5,693,761, 5,693,762, and 6,180,370.

Generally, the transplantation of murine (or other non-human) CDRs onto a human antibody is achieved as follows. The cDNAs encoding heavy and light chain variable domains are isolated from a hybridoma. The DNA sequences of the variable domains, including the CDRs, are determined by sequencing. The DNAs encoding the CDRs are transferred to the corresponding regions of a human antibody heavy or light chain variable domain coding sequence by site directed mutagenesis. Then human constant region gene segments of a desired isotype (e.g, .gamma.1 for CH and kappa. for CL) are added. The humanized heavy and light chain genes are co-expressed in mammalian host cells (e.g., CHO or NSO cells) to produce soluble humanized antibody. To facilitate large scale production of antibodies, it is often desirable to produce such humanized antibodies in bioreactors containing the antibody-expressing cells, or to produce transgenic mammals (e.g., goats, cows, or sheep) that express the antibody in milk (see, e.g., U.S. Pat. No. 5,827,690).

At times, direct transfer of CDRs to a human framework leads to a loss of antigen-binding affinity of the resultant antibody. This is because in some cognate antibodies, certain amino acids within the framework regions interact with the CDRs and thus influence the overall antigen binding affinity of the antibody. In such cases, “back mutations” (supra) should be introduced into the framework regions of the acceptor antibody in order to retain the antigen-binding activity of the cognate antibody.

The general approach of making back mutations is known in the art. For instance Queen, et al., 1989, Proc. Natl. Acad. Sci. USA 86:10029-33, Co et al., 1991, Proc. Natl. Acad. Sci. USA 88:2869-73, and WO 90/07861 (Protein Design Labs Inc.) describe an approach that involves two key steps. First, the human V framework regions are chosen by computer analysis for optimal protein sequence homology to the V region framework of the cognate murine antibody. Then, the tertiary structure of the murine V region is modeled by computer in order to visualize framework amino acid residues that are likely to interact with the murine CDRs, and these murine amino acid residues are then superimposed on the homologous human framework.

Under this two-step approach, there are several criteria for designing humanized antibodies. The first criterion is to use as the human acceptor the framework from a particular human immunoglobulin that is usually homologous to the non-human donor immunoglobulin, or to use a consensus framework from many human antibodies. The second criterion is to use the donor amino acid rather than the acceptor if the human acceptor residue is unusual and the donor residue is typical for human sequences at a specific residue of the framework. The third criterion is to use the donor framework amino acid residue rather than the acceptor at positions immediately adjacent to the CDRs.

One may also use a different approach as described in, e.g., Tempest, 1991, Biotechnology 9: 266-71. Under this approach, the V region frameworks derived from NEWM and REI heavy and light chains, respectively, are used for CDR-grafting without radical introduction of mouse residues. An advantage of using this approach is that the three-dimensional structures of NEWM and REI variable regions are known from X-ray crystallography and thus specific interactions between CDRs and V region framework residues can be readily modeled.

A humanized antibody employed in this invention may contain a mutation (e.g., deletion, substitution or addition) at one or more (e.g., 2, 3, 4, 5, 6, 7 or 8) of certain positions in the heavy chain such that an effector function of the antibody (e.g., the ability of the antibody to bind to a Fc receptor or a complement factor) is altered without affecting the antibody's ability to bind to FANCI (U.S. Pat. No. 5,648,260). These heavy chain positions include, without limitation, residues 234, 235, 236, 237, 297, 318, 320 and 322 (EU numbering system). The humanized antibody can, for instance, contain the mutations L234A (i.e., replacing leucine at position 234 of an unmodified antibody with alanine) and L235A (EU numbering system) in its heavy chain.

In addition, the humanized antibody employed in this invention may contain a mutation (e.g., deletion or substitution) at an amino acid residue that is a site for glycosylation, such that the glycosylation site is eliminated. Such an antibody may be clinically beneficial for having reduced effector functions or other undesired functions while retaining its FANCI binding affinity. Mutations of glycosylation sites can also be beneficial for process development (e.g., protein expression and purification). For instance, the heavy chain of the antibody may contain the mutation N297Q (EU numbering system) such that the heavy chain can no longer be glycosylated at this site.

In still other embodiments, the heavy and/or light chains of the antibody used in this invention contain mutations that increase affinity for binding to FANCI and thereby increase potency for treating FANCI-mediated disorders.

The monoclonal antibodies of this invention may further include other moieties to effect or enhance a desired function. For instance, the antibodies may include a toxin moiety (e.g., tetanus toxoid or ricin) or a radionuclide (e.g., .sup.111 In or .sup.90Y) for killing of cells targeted by the antibodies (see, e.g., U.S. Pat. No. 6,307,026). The antibodies may include a moiety (e.g., biotin, fluorescent moieties, radioactive moieties, histidine tag or other peptide tags) for easy isolation or detection. The antibodies may also include a moiety that can prolong their serum half life, for example, a polyethylene glycol (PEG) moiety, and a member of the immunoglobulin super family or fragment thereof (e.g., a portion of human IgG1 heavy chain constant region such as the hinge, CH2 and CH3 regions).

Antibody fragments and univalent antibodies may also be used in the methods and compositions of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′) as well as tetramers which correspond to the two branch segments of the antibody Y (commonly known as F(ab)₂) whether any of the above are covalently or non-covalently aggregated, so long as the aggregation is capable of specifically reacting with a particular antigen or antigen family.

### 2. Detection Using GFP-FANCI Fusion Proteins

An alternative approach for the detection of FANC I activation and foci formation is the use of a FANC I protein fused with a fluorescent protein, for example, GFP. A functional fusion protein of FANC I and GFP is able to form foci upon exposure to genotoxic anti-neoplastic agents. These foci are then visible by fluorescence microscopy. Therefore, formation of FANCI-containing foci can be measured as a surrogate marker for activation of the FA pathway in response to exposure to genotoxic anti-neoplastic agents. Methods of generating such fusion protein constructs, as well as methods for detecting formation of FANCI-containing foci can be performed using the methods described herein, as well as via adaptation of the methods previously applied to FANC D2 as described in U.S. App. No. 60/540,380, which is incorporated herein by reference in its entirety.

### 3. Detection Using FANCI-Binding Ligands Specific for Ubiquitinated (Activated) FANCI

The total cellular level of FANCI protein does not significantly change in response to DNA damage. Rather, DNA damage results in monoubiquitination of FANCI, as well as recruitment into FANCI-containing foci. It will be appreciated by one skilled in the art that an alternative to measuring the presence of FANCI-containing foci is to use a ligand which specifically binds the monoubiquitinated, but not the unubiquitinated form of FANCI. To detect the presence of monoubiquitinated FANCI, the ligand is preferably associated with a detectable label as described above. The main advantage of using such a ligand, as will be appreciated by one skilled in the art, is that, due to the typically low basal level of monoubiquitinated FANCI in cells with undamaged DNA, the level of FANCI-containing foci can be measured in a sample taken from a living subject using the level of monoubiquitinated FANCI as a surrogate marker. An antibody which specifically recognizes the monoubiquitinated form of FANCI (FANCI-L) has considerable utility as a rapid diagnostic. For instance, this antibody could be used for:
1) Immunohistochemistry (1H). This antibody could be used to examine tissue sections prepared from solid tumors (e.g., breast, ovarian, lung tumors). A positive signal by IH would predict that the tumor will be resistant to cisplatin and related drugs.
2) FACS analysis. Peripheral blood lymphocytes (PBLs) could be screened with this antibody. A positive signal suggests the presence of activated FANCI, consistent with a recent exposure of an individual to IR. or toxin. Thus, this antibody is a useful extension of the radiation dosimeter assay described in this application.
3) A high-throughput assay to screen for inhibitors of the purified FA complex. These inhibitors will block the ability of the FA complex to monoubiquitinate FANCI in vitro. The new monoclonal antibody will be a useful reagent for end product detection. Additional methods of measuring FANCI-containing foci using a ligand which specifically recognizes monoubiquitinated FANCI include immunoblot analysis, or Enzyme linked immunosorbant assays (ELISA) using extracts of samples collected from living subjects, or FACS analysis (Harlow et al, 1999, Using Antibodies: A Laboratory Manual, Cold Spring Harbor Laboratory Press, NY).

A sensitive measure of IR exposure is the increased monoubiquitination of FANCI. In undamaged cells, the ratio of FANCI-L (monoubiquitinated isoform) to FANCI-S (unubiquitinated isoform) is approximately 0.5-0.6. This ratio (L/S) is readily calculated by comparing the density of the L band to the S band on a western blot. A sensitive indicator of increased FANCI monoubiquitination and IR exposure is the conversion of the L/S ratio to 1.0 or greater.

### IV. IDENTIFYING INHIBITORS OF THE FA PATHWAY

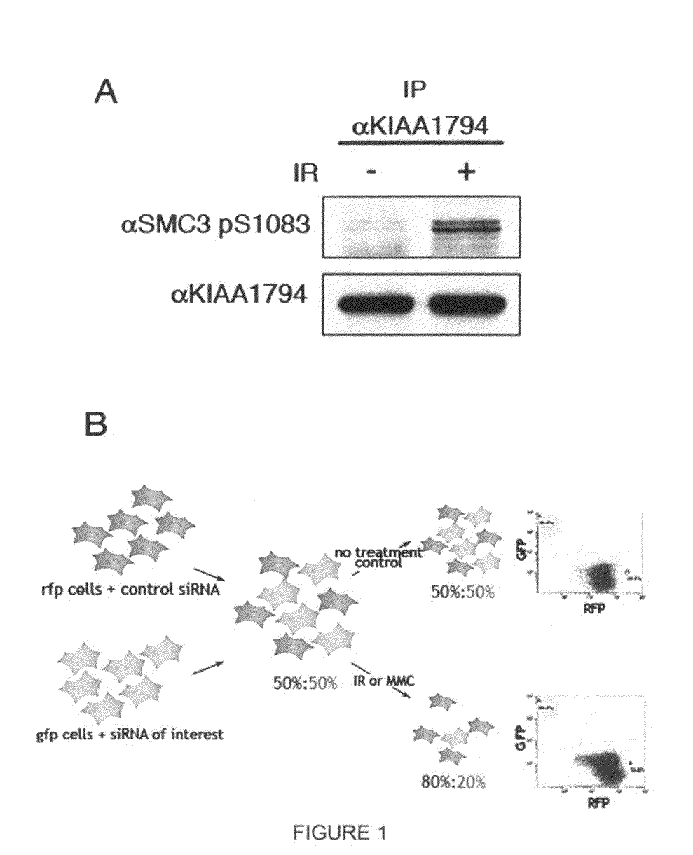

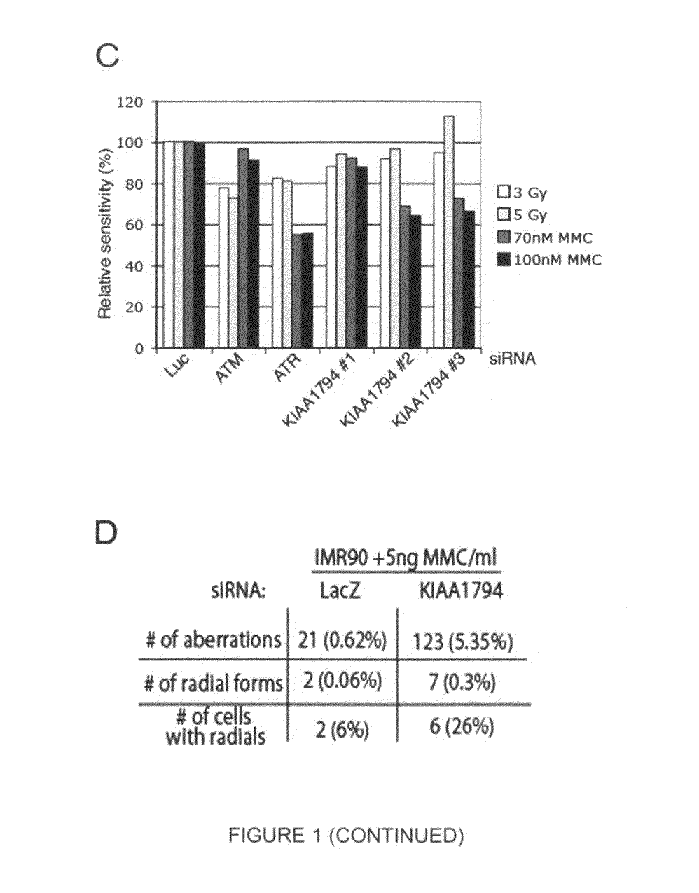

The present invention encompasses methods and compositions useful for the treatment of neoplastic diseases using inhibitors of the FA pathway. Inhibitors of the FA pathway can be identified by methods described herein, and also methods previously described, for example, in U.S. application Ser. No. 10/165,099 and U.S. App. No. 60/540,380, the contents of which are incorporated herein by reference. For example, inhibitors of the FA pathway can be identified systematically using a three-tiered approach, as summarized in FIG. 1 of U.S. App. No. 11,441,289, the contents of which is incorporated herein by reference.

The first tier of screening comprises a high-throughput method to identify agents which alter the formation of FANCI-containing foci. Detection of FANCI-containing foci, for example by using a FANCI ligand such as anti-FANCI antibodies or cell lines expressing a functional eGFP-FANCI fusion protein, can be performed as described for FANC D2 in U.S. application Ser. No. 10/165,099 and U.S. App. No. 60/540,380, the contents of which are incorporated herein by reference. The method comprises contacting cells or a biological sample with a test compound simultaneously with, before or after exposure to a genotoxic anti-neoplastic agent, for example ionizing radiation (IR), mitomycin C or cisplatin, at a dosage which induces formation of FANCI-containing foci. The number and size FANCI-containing foci are then detected in cells and compared with control cells which were not contacted with the test compound. A decrease in the number and/or size of FANCI-containing foci relative to control cells is indicative of an agent which is an inhibitor of the FA pathway, whereas an increase in the number and/or size of FANCI-containing foci relative to control cells is indicative of an agent which is an agonist of the FA pathway. Potential agonists and inhibitors thus identified can be further tested to determine whether they exert their effects directly on the FA pathway, or act indirectly, for example, by directly causing damage to DNA (in the case of potential agonists of the FA pathway), or by reducing the effect of the genotoxic anti-neoplastic agent that was used in the screen.

The second tier of screening involves the detection of ubiquitinated FANCI polypeptides. As disclosed herein, activation of the FA pathway results in monoubiquitination of the FANCI polypeptide. Activation of the FA pathway can therefore be measured by detecting the relative amount of ubiquitinated FANCI compared with unubiquitinated FANCI polypeptide. The ubiquitination of FANCI can be detected by performing immunoblot analysis of protein extracts. Ubiquitinated FANCI migrates at a higher molecular weight band on immunoblot analyses, and can be detected using a labeled FANCI ligand, for example an anti-FANCI antibody. Therefore, the second tier of the screening comprises contacting cells or a biological sample with a test compound simultaneously with, before or after exposure to a genotoxic anti-neoplastic agent, for example ionizing radiation (IR), mitomycin C or cisplatin, at a dosage which induces formation of FANCI-containing foci. The amount of ubiquitinated FANCI polypeptide relative to unubiquitinated FANCI polypeptide is detected, and compared with samples from control cells or biological samples which were not contacted with the test compound. A difference in the relative amount of ubiquitinated FANCI relative to control cells indicates that the test compound is a modulator of the FA pathway. An increase in the relative amount of ubiquitinated FANCI polypeptide compared with control cells or biological samples is indicative of an agonist of the FA pathway, whereas a decrease in the relative amount of ubiquitinated FANCI polypeptide compared with control cells or biological samples is indicative of an inhibitor of the FA pathway. As described previously, the potential agonists and inhibitors thus identified can be further tested to determine whether they exert their effects directly on the FA pathway, or act indirectly, for example, by directly causing damage to DNA (in the case of potential agonists of the FA pathway), or by reducing the effect of the genotoxic anti-neoplastic agent that was used in the screen.

The third tier of screening comprises in vitro testing of compounds for sensitivity to genotoxic anti-neoplastic agents. Contacting cells or biological samples with inhibitors of the FA pathway would be expected to increase the sensitivity of the samples/cells to genotoxic anti-neoplastic agents. Specific inhibition of the FA pathway by a test agent is expected to increase the sensitivity to a degree comparable to, for example, a cell line with a specific defect in one or more components of the FA pathway. Cell lines useful for this type of assay include the ovarian cancer cell line, 2008, which is deficient in FANCF. 2008 cells deficient in FANCF show heightened sensitivity to genotoxic anti-neoplastic agents, as described, e.g., in U.S. application Ser. No. 11/441,289, and this sensitivity is restored to wild-type levels by overexpression of the FANCF. The role of FANCF in restoring wild-type levels of genotoxin sensitivity is then abolished by contacting with a test agent which inhibits the FA pathway, while leaving the sensitivity to the genotoxic anti-neoplastic agent unaffected in the absence of the FANCF transfection.

The three tiers of screening described above provide a stream-lined approach to rapidly identifying and characterizing potential modulators of the FA pathway. It should be understood that methods to identify modulators are not limited to the particular embodiments of the invention described above, and variations of the embodiments can be made and still fall within the scope of the invention. In addition, the terms used herein are for the purpose of describing the particular embodiments and are not intended to be limiting.

### V. INHIBITORS OF THE FA PATHWAY

The present invention contemplates the use of inhibitors of the FA pathway. An inhibitor of the FA pathway includes any compound which results in the inhibition of formation of FANCI-containing foci, when administered before, after or concomitantly with a genotoxic anti-neoplastic agent(s) which normally cause formation of FANCI-containing foci. Examples of genotoxic anti-neoplastic agents which induce formation of FANCI-containing foci include, but are not limited to, ionizing radiation (IR) and DNA alkylating agents such as cisplatin or mitomycin C. Inhibition of the FA pathway can also be detected by measuring the relative amounts of ubiquitinated and unubiquitinated FANCI polypeptide of samples subjected to an agent which normally induces ubiquitination. Detection of FANCI-containing foci using, for example, microscopic detection means, as well as determination of the relative ubiquitination state of the FANCI polypeptide, can be performed as described for detection of FANC D2-containing foci in U.S. Ser. No. 10/165,099, filed Jun. 6, 2002, and U.S. Ser. No. 60/540,380, filed Jan. 30, 2004, the contents of which are incorporated herein by reference. Briefly, FANCI-containing foci can be detected using immunofluorescence microscopy, using anti-FANCI antibodies. Alternatively, a fluorescent protein-tagged version of FANCI can be transfected into the cells of interest, and formation of FANCI-containing foci measured microscopically be detecting fluorescent ‘foci’, again, as described for FANC D2 in U.S. Ser. No. 60/540,380. Compounds which inhibit the FA pathway, such as wortmannin and Trichostatin A, have previously been disclosed, for example in U.S. Ser. No. 60/540,380, filed Jan. 30, 2004.

### VI. INHIBITORS OF OTHER DNA DAMAGE REPAIR PATHWAYS

Cells are continuously subjected to different kinds of DNA damage. These damages can arise from exposure to a variety of internal and external chemicals and radiation, including reactive oxygen species such as superoxide (O₂⁻), hydrogen peroxide (H₂O₂). In addition, humans are constantly exposed a vast array of carcinogens, many of which act by causing damage to the DNA. It has been shown that at least six distinct mechanisms exist for DNA damage repair in humans, depending upon the type of damage incurred.

Many cancers have a defect in at least one of the six major DNA damage repair pathways. In addition to causing increased genomic instability, disruption of any of these DNA repair mechanisms can lead to increased sensitivity to genotoxic anti-neoplastic agents. Therefore, these cancers have increased dependence on one of the other five DNA damage repair pathways for survival. Hence, disruption of a second, non-FA DNA damage repair pathway in these neoplastic disorders, for example by a small molecule inhibitor may result in selective cancer cell death. Stated differently, many cancers may turn out to have a dominant (primary) DNA damage repair pathway. Since one DNA damage repair pathway is already abolished or significantly reduced in the cancer, an extra burden is placed on the dominant pathway in order to maintain the high proliferation rate and to prevent DNA damage of these cells. Disruption of the dominant pathway in a cancer cell in which a major DNA damage repair pathway is abolished or diminished, by means of an exogenous inhibitor, may therefore have a profound cytotoxic effect on the tumor cells but a relatively small cytotoxic effect on the surrounding normal cells.

Loss of the FA/BRCA pathway leads to chromosome instability, increased cisplatin sensitivity, thus resulting in increased activity of the remaining non-FA DNA damage repair pathways, including the Base Excision Repair (BER) pathway. Accordingly, an inhibitor of a non-FA DNA damage repair pathway, for example, BER (such as a PARP1 inhibitor or an inhibitor of a specific kinase in the BER pathway) would be lethal to those cells, but may have little effect on normal (non-tumor) cells.

The present invention also contemplates the use of inhibitors of various other DNA damage repair pathways. As previously described, there are several major pathways for DNA damage repair, including but not limited to, non-homologous end joining (NHEJ), base excision repair (BER), nucleotide excision repair (NER), and mismatch repair (MR). These mechanisms are described, for example, in Hoeijmakers J H J (2001) Nature 411: 366-374, Svejstrup J Q (2002) Nat Rev Mol Cell Biol. 3: 21-29, and in Panasci, DNA Repair in Cancer Therapy Humana Press, 2004, Totowa, N.J., which are incorporated herein by reference.

A. Non-Homologous End Joining (NHEJ)

DNA double strand breaks (DSBS) can be caused by any number of environmental or other factors, including reactive oxygen species, ionizing radiation (IR) and certain anti-neoplastic drugs like bleomycin. Failure to repair DSBs can lead to a number of consequences, including mutations, chromosomal aberrations, and eventually cell death. Non-homologous end-joining (NHEJ), also called illegitimate recombination, is one major pathway of repairing DSBs. Some members of the NHEJ pathway are shown in Table 1.

TABLE 1
Genes and Proteins Important for NHEJ

| Gene name | Protein name; function |
|---|---|
| MRE11 | Exonuclease (3′ to 5′) |
| NBS1 | Mre11-interaction |
| RAD50 | Role in stimulation of MRE11 exonuclease activity |
| XRCC4 | Unknown function; interacts with DNA ligase IV |
| XRCC5 | Ku80; forms heterodimer with Ku70 which binds to |
| | DS DNA ends and DS/SS DNA junctions |
| XRCC6 | Ku70; forms heterodimer with Ku 70; deficiency |
| | correlated with elevated frequency of T-cell |
| | lymphoma |
| XRCC7 | DNA-protein kinase; regulates Ku heterodimer |

The DNA-dependent protein kinase (DNA-PK) consists of the catalytic subunit (DNA-PKcs) and the regulatory subunit (the Ku70/Ku80 heterodimer). The DNA-PKcs subunit is a serine/threonine kinase which belongs to the phosphatidyl inositol-3 kinase family. The Ku80/Ku70 heterodimer (Ku) exhibits sequence-independent affinity for double-stranded termini and, upon binding to DNA, recruits and activates the DNA-PKcs catalytic subunit. Several candidate inhibitors of the DNA-PK have been described, for example viridins (Hanson, J. R. Nat. Prod. Rep., 12: 381-384, 1995), wortmannin, quercitins (Izzard et al. (1999) Cancer. Res., 59: 2581-2586), LY294002 (Vlahos et al. (1994) J. Biol. Chem., 269: 5241-5248), which are incorporated herein by reference. Other inhibitors of NHEJ include inhibitors of ATM disclosed within U.S. Ser. No. 2004/0002492, which are incorporated herein by reference.

B. Base Excision Repair (BER)

Single Strand DNA breaks (SSBs) are one of the most frequent lesions occurring in cellular DNA. SSBs can occur spontaneously or as intermediates of enzymatic repair of base damage during Base Excision Repair (BER) (Caldecott (2001) Bioessays 23(5): 447-55). In this repair pathway, which follows the removal of a damaged base by a DNA glycosylase, the resulting apurinicu/apyrimidinic (AP) site is processed first by the Ape1 AP endonuclease, leaving a 5′ deoxyribose-phosphate; then by an AP lyase activity leaving a 3′ β-elimination product. The subsequent removal of these AP sites by DNA Polymerase β, or by a PCNA-dependent polymerase, allows the repair synthesis to fill-in either a single nucleotide (for Pol β) or a longer repair patch (for Pol δ/ε), which are then re-ligated (Wilson (1998) Mutat Res. 407:203-15). If SSB sites are not efficiently processed and removed, clusters of damaged sites and stalled replication forks will form, resulting in the formation of DSBs with potentially lethal consequences for the cell (Chaudhry & Weinfeld (1997) J Biol. Chem. 272:15650-5; Harrison, Hatahet et al. (1998) Nucleic Acids Res. 26:932-41).

Poly(ADP-ribose) polymerase (PARP) is a DNA binding zinc finger protein that catalyzes the transfer of ADP-ribose residues from NAD+ to itself and different chromatin constituents, forming branched ADP-ribose polymers. The enzymatic activity of PARP is induced upon DNA damage, suggesting a role of PARP in DNA repair and DNA damage-induced cell death. Numerous inhibitors of PARP have been disclosed, some of which are commercially available. For example, PJ-34 N-(6-oxo-5,6-dihydrophenanthridin-2-yl)-N,N-dimethylacetamide.Hcl, INHBP 5-iodo-6-amino-1,2-benzopyrone, 3-Aminobenzamide, Benzamide, 4-Amino-1,8-naphthalimide, 6(5H)-Phenanthridinone, 5-Aminoisoquinolinone (5-AIQ). hydrochloride, 4-Hydroxyquinazoline, 4-Quinazolinol, 1,5-Isoquinolinediol, 5-Hydroxy-1 (2H)-isoquinolinone, 3,4-Dihydro-5-[4-(1-piperidinyl)butoxy]-1 (2H)-isoquinolinone (DPQ) are all available from Inotek Pharmaceuticals (Beverly, Mass.). Other compounds, such as GPI 15427 (Tentori et al. (2003) Proceedings of the AACR, 44, Abs No. 5466) and methoxyamine (Liuzzi et al., (1985) J. Biol. Chem. 260, 5252-5258; Rosa et al. (1991) Nucleic Acids Res., 19, 5569-5574; and Horton et al. (2000) J. Biol. Chem., 275, 2211-2218) have been reported to enhance the anti-neoplastic efficacy of both chemotherapy and radiation therapy.

C. Nucleotide Excision Repair (NER)

Nucleotide excision repair (NER) acts on a variety of helix-distorting DNA lesions, caused mostly by exogenous sources that interfere with normal base pairing. The primary function of NER in man appears to be the removal of damage, for example pyrimidine dimers, which are induced by ultraviolet light (UV). Members of the NER pathway, defects of which can cause an autosomal recessive disease called xeroderma pigmentosum (XP), have been identified, including seven different genes, XPA, XPB, XPC, XPD, XPE, XPF and XPG, all of which function in the NER pathway (Hoeijmakers (2001) Mutat Res. 485:43-59).

Eukaryotic NER includes two major branches, transcription-coupled repair (TCR) and global genome repair (GGR) (de Laat et al. (1999) Genes Dev. 13:768-85, Tomaletti & Hanawalt (1999) Biochimie. 81:139-46). GGR is a slow random process of inspecting the entire genome for injuries, while TCR is highly specific and efficient and concentrates on damage-blocking RNA polymerase II. The two mechanisms differ in substrate specificity and recognition. In GGR, the XPC—HR23B complex recognizes damage located in nontranscribed regions (Sugasawa et al. (2001) Genes Dev. 15:507-21), whereas the arrest of RNA polymerase II (RNAPII) serves as the recognition signal in TCR. The molecular mechanism of RNAPII displacement is currently unclear, but essential factors, such as the Cocayne's syndrome proteins CSA, CSB, XPA-binding protein 2 (XAB2), TFIIH and XPG (Svejstrup 2002), have been identified to function in TCR. Subsequently, both in GGR and TCR, an open unwound structure forms around the lesion. This creates specific cutting sites for XPG and ERCC1-XPF nucleases, and the resulting gap is filled in by PCNA-dependent polymerase and sealed by DNA ligase (de Laat et al., id).

D. Mismatch Repair (MR)

Mismatch repair (MMR) removes both nucleotides mispaired by DNA polymerases and insertion/deletion loops caused by slippage during replication of repetitive sequences (Harfe & Jinks-Robertson (2000) Annu Rev Genet. 34: 359-399). Initially, the heterodimeric MSH complex recognizes the nucleotide mismatch, subsequently followed by interaction with MLH1/PMS2 and MLH1/MLH3 complexes. Several proteins participate in process of the nucleotide excision and resynthesis. Tumor cells deficient in mismatch repair have much higher mutation frequencies than normal cells (Parsons et al. (1993) Cell 75: 1227-1236, Bhattacharyya et al. (1994) Proc Natl Acad. Sci. USA 91: 6319-6323). At least six genes MSH2, MLH1, PMS2, MSH3, MSH6 and MLH3 have been identified in humans which are involved in mismatch repair. Defects in these genes except for MSH3 leads to hereditary nonpolyposis colon cancer (HNPCC) (Hoeijmakers 2001).

Other inhibitors to DNA damage repair have been disclosed, including aphidicolin, (Gera (1993) J Immunol. 151:3746-57), rapamycin (mTOR inhibitor, Sabers et al., (1995) J. Biol. Chem. 270:815-22), the AGT inhibitor 06-benzylguanine (Bronstein et al., (1992) Cancer Res. 52:3851-6).

### VII. IDENTIFYING INHIBITORS OF NON-FA DNA DAMAGE REPAIR PATHWAYS

As previously described, in certain situations the DNA damage repair pathways of the cell can be partially redundant. This presents difficulties in identifying agents which specifically block one pathway. Inhibitors identified using cell-based methods wherein the cells have functional DNA damage repair pathways may therefore have multiple targets, including in a plurality of DNA damage repair pathways. Therefore, use of cell lines deficient in one or more DNA damage repair pathways may greatly accelerate the identification of novel, specific inhibitors. Therefore, according to one aspect, a method of identifying agents which inhibit a non-FA DNA damage repair pathway is provided. The method employs cells which have a lesion in the FA pathway. The method comprises contacting cells with an agent, and testing for sensitivity to a genotoxic anti-neoplastic agent. An agent which confers enhanced sensitivity to the genotoxic anti-neoplastic agent in test cells containing a lesion in the FA pathway when compared with control cells containing functional DNA damage repair pathways indicates that the agent inhibits a non-FA DNA damage repair pathway other than the pathway in which the test cell contains a lesion. In one embodiment, test and control cells are isogenic, except that the test cell contains a lesion in at least one component of the FA/BRCA pathway, for example, in FANCA, FANCB, FANCC, FANCD, FANC D2, FANCE, FANCF, FANCG, FANCL, and the ATR protein kinase, among others. (It is noted that ATR appears to directly regulate the FA pathway. ATR is required for monoubiquitination of FANCD2 (Andreassen et al. (2004) Genes Dev 18: 1958-1963) and phosphorylates FANCD2 on several sites required for FANCD2 function (Ho et al. (2006) Mol Cell Biol 26: 7005-7015; Taniguchi et al. (2002) Cell 109: 459-472).)

According to one embodiment, the method comprises comparing the sensitivities to genotoxic anti-neoplastic agents of two isogenic cell lines which differ in the functionality of the FA pathway. The availability of isogenic cell lines also permits the identification of gene products which are involved in DNA damage repair pathways other than the FA pathway. In one embodiment, genes affecting the viability of the parental but not the control cells are tested by systematic, mass inhibition using an siRNA library. For example, a bar-coded siRNA library can be used to for stable transfection of the two cell lines. Genes that are required for viability of the 2008 cells, but not for the corrected cells. Genes which are important for DNA damage repair pathways other than the FA pathway, for example in the BER pathway, is expected to have the result that siRNA knockdown of such a gene will be lethal in the parental 2008 cells, but not in the control 2008 cells which have been transfected with the FANCF cDNA.

Agents thus identified which can kill a cell in which one or more DNA damage repair pathways is disrupted but do not kill an isogenic cell line in which the disruption is restored can be used in the treatment of cancer. Disruption of two or more of the six major DNA damage repair pathways can result in cell death. Since many cancers already have the one pathway knocked out or repressed, a relatively non-toxic inhibitor of the second pathway, for example the BER pathway, may be sufficient to cause cytoreduction of the cancer, even in the absence of a chemotherapeutic agent. In addition, in tumors cells in which the major DNA damage repair pathways are intact, using two inhibitors in combination (e.g., one inhibitor of the FA pathway and one inhibitor of the BER pathway) may be sufficient to cause significant cytoreduction, provided that the toxicity of such a combination is not toxic to normal (non-cancer) cells. In such a case, a pro-drug strategy to enhance uptake of these agents by cancer cells provide the necessary therapeutic index.

### VIII. ANTI-NEOPLASTIC AGENTS

Disclosed herein are methods of treating patients with neoplastic disorders using a combination of anti-neoplastic agents in combination with inhibitors of DNA damage repair pathways. Anti-neoplastic agents which are particularly useful include, but are not limited to, agents which cause damage to the DNA. These agents include DNA alkylating agents, intercalating agents, and the like. Further contemplated, therefore, is the use of DNA-damaging chemotherapeutic compounds including, but not limited to, 1,3-Bis(2-Chloroethyl)-1-NitrosoUrea (BCNU), Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolomide, and Topotecan. Furthermore, methods described herein can also employ radiotherapeutic methods of treating neoplastic disorders. In one embodiment, the genotoxic anti-neoplastic agents do not inhibit DNA damage repair at the concentrations administered.

### IX. IDENTIFYING RESPONDERS TO ANTI-NEOPLASTIC AGENTS

The efficacy of the FA pathway of a cell has been identified to strongly correlate with the cell's sensitivity to chemotherapeutic agents. Therefore, in one aspect, the invention provides a method of predicting whether a subject with a neoplastic disorder or disease will respond to a genotoxic anti-neoplastic agent. The method comprises obtaining a biological sample from the subject, and determining the localization (e.g., determining size and/or number of FANCI-containing foci) and/or degree of ubiquitination of FANCI polypeptide within the biological sample. A degree of ubiquitination of the FANCI polypeptide in the biological sample of the subject that is reduced (e.g., less than about 70%, less than about 50%, etc.) when compared with a biological sample from a control subject is indicative of a subject that will respond to a genotoxic anti-neoplastic agent. Similarly, a reduction in size and/or number of FANCI-containing foci when compared to control cells is also indicative of a subject that will respond to a genotoxic anti-neoplastic agent.

In another aspect, the invention provides a method of predicting whether a subject with a neoplastic disorder or disease will respond to a genotoxic anti-neoplastic agent that employs examination of FANCI sequence in a biological sample. The method comprises obtaining a biological sample from the subject, and determining the FANCI nucleic acid and/or polypeptide sequence within the biological sample. The finding of mutations, especially, e.g., functional coding sequence changes such as the R1285Q mutation, within a biological test sample as compared to control sequence is indicative of a subject that will respond to a genotoxic anti-neoplastic agent.

In one embodiment, the neoplastic disorder is selected from the group consisting of leukemia, acute myeloid leukemia, chronic myeloid leukemia, chronic lymphatic leukemia, myelodysplasia, multiple myeloma, Hodgkin's disease or non-Hodgkin's lymphoma, small or non-small cell lung carcinoma, gastric, intestinal or colorectal cancer, prostate, ovarian or breast cancer, head, brain or neck cancer, cancer in the urinary tract, kidney or bladder cancer, malignant melanoma, liver cancer, uterine or pancreatic cancer.

According to these aspects, the ability of a biological sample to activate the FA pathway, as determined by measuring the level of FANCI monoubiquitination, localization, nucleic acid and/or polypeptide sequence is determined to identify responders to chemotherapeutic agents, particularly genotoxic anti-neoplastic agents. The anti-neoplastic agents can be any which are used for the treatment of cancer, and in one embodiment, anti-neoplastic agents' mechanism of action is through the damage of DNA. These compounds include but are not limited to: 1,3-Bis(2-Chloroethyl)-1-NitrosoUrea (BCNU), Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolomide, and Topotecan and ionizing radiation.

In certain embodiments the subject or, alternatively, the biological sample obtained from the subject, can be exposed to the anti-neoplastic agent prior to determining the degree of ubiquitination of the FANCI polypeptide. In one embodiment, the subject or biological sample obtained from the subject is exposed at a dose that is less than or equal to the therapeutically effective dose. In another embodiment, the exposure is at 50% or less of the therapeutically effective dose of the anti-neoplastic agent.

The degree of ubiquitination of the FANCI polypeptide can be compared with that of a control subject. As used herein, a control subject can be a single subject that has previously been determined to be normal with respect to response to anti-neoplastic agents, or a number of normal subjects. Biological samples from either a single control subject or a number of control subjects can be used. In this aspect, a subject is deemed to be a responder to an anti-neoplastic agent if the percentage of FANCI ubiquitination is reduced when compared with a sample from a subject, for example, less than about 70%, less than 65%, less than 60%, less than 50%, less than 40%, less than 30%, less than 20%, less than 10% or less, when compared with a sample from a subject that has received the same or equivalent dose of anti-neoplastic agent as the test sample. Furthermore, in embodiments involving exposure to an anti-neoplastic agent prior to determining the degree of ubiquitination and/or localization of the FANCI polypeptide, control samples can be prepared prior to preparation of the test samples, or prepared simultaneously to preparation of the test samples.

In one embodiment, the subject, or alternatively the biological sample taken from the subject, can be treated with a genotoxic anti-neoplastic agent prior to measurement of the efficacy of the FA pathway. The dosage of the anti-neoplastic agent would be that necessary to induce the FA pathway in a normal subject. Typically, the dosage of the anti-neoplastic agent would be from between about 5% to 100% of the typical therapeutically effective dose, more typically between 20% to 100%, and most typically between about 35%-100%.

As described herein, there are a number of ways in which to measure the degree of ubiquitination and/or localization of the FANCI polypeptide in biological samples. The degree of ubiquitination of the FANCI polypeptide can be measured using immunoblot analysis as described herein and as previously described for FANC D2. Alternatively, one can detect the formation of FANCI-containing foci, for example using immunofluorescence microscopy of biological samples, as a surrogate marker for FANCI ubiquitination.

Subjects are considered responders if the formation of ubiquitinated FANCI polypeptide is significantly reduced, e.g., if the formation of ubiquitinated FANCI is about 70% or less when compared with normal subjects, 65% or less, 60% or less, 50% or less, 40% or less, 30% or less than in normal subjects.

### X. TREATMENT OF NEOPLASTIC DISORDERS

In certain embodiments, a subject or patient is administered with a therapeutically effective dose of a genotoxic anti-neoplastic agent, simultaneously, before or after administration with an inhibitor of a non-FA DNA damage repair pathway. Therapeutically effective dosages of many anti-neoplastic agents are well-established, and can be found, for example, in Cancer Chemotherapy and Biotherapy: A Reference Guide Edition Number: 2 Tenenbaum, ed. Saunders & CO (1994) which is incorporated herein by reference.

Also provided herein are methods for treating a neoplastic disorder in a subject in need thereof. In one aspect, the method comprises administering to the subject an effective amount of an inhibitor of FANCI and/or the FA pathway and a genotoxic anti-neoplastic agent. The anti-neoplastic agent can be selected from the group consisting of 1,3-Bis(2-Chloroethyl)-1-NitrosoUrea (BCNU), Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolomide, and Topotecan and ionizing radiation.

In another aspect, a method of treating a neoplastic disorder in a subject in need thereof is provided. The method comprises administering to the subject an effective amount of an inhibitor of FANCI and/or the FA pathway and an inhibitor of a non-FA DNA damage repair pathway. The inhibitor of a non-FA DNA damage repair pathway can be selected which inhibits any of the repair pathways, and can be selected from the group consisting of PARP inhibitors, DNA-PK inhibitors, mTOR inhibitors, ERCC1 inhibitors ERCC3 inhibitors, ERCC6 inhibitors, ATM inhibitors, XRCC4 inhibitors, Ku80 inhibitors, Ku70 inhibitors, XPA inhibitors, CHK1 inhibitors, CHK2 inhibitors, or pharmaceutically acceptable salts, esters, derivatives, solvates or prodrugs thereof. The inhibitor of FANCI and/or the FA pathway can be administered before, simultaneously with, or after administration of the inhibitor of the non-FA DNA damage repair pathway. The inhibitors can be administered parenterally, orally or directly into the tumor.

The inhibitor of FANCI and/or the FA pathway, as well as inhibitor of a non-FA DNA damage repair pathway, can act to increase the sensitivity of a neoplastic disorder to a genotoxic anti-neoplastic agent. Therefore, in another aspect, a method of increasing the sensitivity of a neoplastic disorder to a genotoxic anti-neoplastic agent is provided. The method comprises administering before, after or concurrently with a therapeutically effective dose of the agent a combination of an effective amount of an inhibitor of FANCI and/or the FA pathway and an inhibitor of a non-FA DNA damage repair pathway. The method can be useful for the treatment of many types of neoplastic disorders, and can be selected from the group consisting of leukemia, acute myeloid leukemia, chronic myeloid leukemia, chronic lymphatic leukemia, myelodysplasia, multiple myeloma, Hodgkin's disease or non-Hodgkin's lymphoma, small or non-small cell lung carcinoma, gastric, intestinal or colorectal cancer, prostate, ovarian or breast cancer, head, brain or neck cancer, cancer in the urinary tract, kidney or bladder cancer, malignant melanoma, liver cancer, uterine or pancreatic cancer.

Inhibitors of FANCI and/or the FA pathway are further useful as agents which increase the sensitivity of a neoplastic disorder to a genotoxic anti-neoplastic agent. Therefore, in another aspect, the invention provides a method of increasing the sensitivity of a neoplastic disorder to a genotoxic anti-neoplastic agent. The method comprises administering before, after or concurrently with a therapeutically effective dose of an genotoxic anti-neoplastic agent, an effective amount of an inhibitor of FANCI and/or the FA pathway. As previously described, the inhibitor of FANCI and/or the FA pathway can be administered before, simultaneously with, or after administration of the inhibitor of the non-FA DNA damage repair pathway, and can be administered parenterally, orally or directly into the tumor. In one embodiment, the method further comprises administering an inhibitor of a non-FA DNA damage repair pathway, in addition to the FANCI and/or FA inhibitor and genotoxic anti-neoplastic agent. The inhibitor of the non-FA DNA damage repair pathway can be administered before, after, or concurrently with a therapeutically effective dose of the FANCI and/or FA pathway inhibitor and genotoxic anti-neoplastic agent.

The efficacy of compositions disclosed herein in preventing or treating neoplastic disorders can be tested, for example, in animal models of specific neoplastic disorders. Numerous examples of animal models are well known to those skilled in the art, and are disclosed, for example, in Holland, Mouse Models of Cancer (Wiley-Liss 2004); Teicher, Tumor Models in Cancer Research (Humana Press; 2001); Kallman, Rodent Tumor Models in Experimental Cancer Therapy (Mcgraw-Hill, Tex., 1987); Hedrich, The Laboratory Mouse (Handbook of Experimental Animals) (Academic Press, 2004); and Arnold and Kopf-Maier, Immunodeficient Animals: Models for Cancer Research (Contributions to Oncology, Vol 51) (Karger, 1996), the contents of which are incorporated herein in their entirety.

### XI. TEST COMPOUNDS ACCORDING TO THE INVENTION

Whether in an in vitro or in vivo system, the invention encompasses methods by which to screen compositions which can inhibit the formation of FANCI-containing foci, as well as compositions which inhibit DNA damage repair pathways other than the FA pathway. Candidate modulator compounds from large libraries of synthetic or natural compounds can be screened. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Alternatively, libraries of natural compounds in the form of bacterial, fungal, plant and animal extracts are available from e.g., Pan Laboratories (Bothell, Wash.) or MycoSearch (NC), or are readily producible by methods well known in the art. Additionally, natural and synthetically produced libraries and compounds are readily modified through conventional chemical, physical, and biochemical means.

Useful compounds may be found within numerous chemical classes, though typically they are organic compounds, including small organic compounds. Small organic compounds have a molecular weight of more than 50 yet less than about 2,500 Daltons, preferably less than about 750, more preferably less than about 350 Daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g., for the amino group, acylation or alkylation, and for the carboxyl group, esterification or amidification, or the like.

Candidate modulators which may be screened according to the methods of the invention include receptors, enzymes, ligands, regulatory factors, and structural proteins. Candidate modulators also include nuclear proteins, cytoplasmic proteins, mitochondrial proteins, secreted proteins, plasmalemma-associated proteins, serum proteins, viral antigens, bacterial antigens, protozoan antigens and parasitic antigens. Candidate modulators additionally comprise proteins, lipoproteins, glycoproteins, phosphoproteins and nucleic acids (e.g., RNAs such as ribozymes, RNAi agents, or antisense nucleic acids). Proteins or polypeptides which can be screened using the methods of the present invention include hormones, growth factors, neurotransmitters, enzymes, clotting factors, apolipoproteins, receptors, drugs, oncogenes, tumor antigens, tumor suppressors, structural proteins, viral antigens, parasitic antigens, bacterial antigens and antibodies (see below).

Candidate modulators which may be screened according to the invention also include substances for which a test cell or organism might be deficient or that might be clinically effective in higher-than-normal concentration as well as those that are designed to eliminate the translation of unwanted proteins. Nucleic acids of use according to the invention not only may encode the candidate modulators described above, but may eliminate or encode products which eliminate deleterious proteins. Such nucleic acid sequences are RNAi agents, antisense RNA and ribozymes, as well as DNA expression constructs that encode them. Note that antisense RNAi agents, RNA molecules, ribozymes or genes encoding them may be administered to a test cell or organism by a method of nucleic acid delivery that is known in the art, as described below. Inactivating nucleic acid sequences may encode a ribozyme; RNAi agent, or antisense RNA specific for the target mRNA. Ribozymes of the hammerhead class are the smallest known, and lend themselves both to in vitro production and delivery to cells (summarized by Sullivan, (1994) J. Invest. Dermatol., 103: 85S-98S; Usman et al., (1996), Curr. Opin. Struct. Biol., 6: 527-533).

### XII. PHARMACEUTICAL COMPOSITIONS

In another aspect, the invention relates to methods and pharmaceutical compositions comprising an inhibitor of FANCI and/or the FA pathway in combination with an anti-neoplastic agent and/or inhibitor of a non-FA DNA damage repair pathway, as described in the preceding section, and a pharmaceutically acceptable carrier, as described below. The pharmaceutical composition comprising an inhibitor of the FANCI and/or the FA pathway is useful for treating a variety of diseases and disorders including cancer, and may be useful as protective agents against genotoxic anti-neoplastic agents.

In one embodiment, the invention provides for a method of treating a neoplastic disorder in a subject in need thereof comprising administering a combination of an effective amount of:

a) an inhibitor of FANCI or pharmaceutically acceptable salts, esters, derivatives, solvates or prodrugs thereof, and

b) a genotoxic anti-neoplastic agent.

Examples of inhibitors of FANCI include the siRNA molecules disclosed herein. Previously identified inhibitors of the FA pathway include, e.g., H-9, alsterpaullone and curcumin. However, it will be appreciated by those skilled in the art that additional inhibitors of FANCI and/or the FA pathway can be identified, for example, using the methods described herein. In this regard, an inhibitor of FANCI and/or the FA pathway can be a small molecule, and antibody, a ribozyme or RNAi agent (e.g., siRNA molecule).

The method can be used in the treatment of various neoplastic disorders, including leukemia, acute myeloid leukemia, chronic myeloid leukemia, chronic lymphatic leukemia, myelodysplasia, multiple myeloma, Hodgkin's disease or non-Hodgkin's lymphoma, small or non-small cell lung carcinoma, gastric, intestinal or colorectal cancer, prostate, ovarian or breast cancer, head, brain or neck cancer, cancer in the urinary tract, kidney or bladder cancer, malignant melanoma, liver cancer, uterine or pancreatic cancer. In one embodiment, the method is used to treat ovarian cancer.

The dosage of the inhibitor of FANCI and/or the FA pathway depends on several factors, including solubility, bioavailability, plasma protein binding, kidney clearance, and inhibition constants. In certain therapeutic applications, an adequate amount to accomplish at least partial inhibition of FANCI and/or the FA pathway is defined as an “effective dose”. Amounts needed to achieve this dosage will depend upon the severity of the disease and the general state of the patient's own immune system, but generally range from 0.005 to 5.0 mg of the inhibitorper kilogram of body weight, with doses of 0.05 to 2.0 mg/kg/dose being more commonly used. Alternatively, the dosage can be administered using a functional dosage, since the activation of FANCI and/or the FA pathway in a subject can be determined empirically using the ubiquitination of the FANCI polypeptide using the methods described herein. Additionally and/or alternatively, the activation state, e.g., ubiquitination state and/or localization, of FANC D2 can be used to assess activation of the FA pathway. Therefore, an “effective dose” of an inhibitor of FANCI and/or the FA pathway can mean a dose required to reduce the level of FANCI ubiquitination to about 70% or less when compared with a control sample, more typically to about 50% or less than a control sample. In this regard, a control sample is ideally taken from the same subject, before administration of the inhibitor.

The dosage of the inhibitor of FANCI and/or the FA pathway in relation to the dosage of the genotoxic anti-neoplastic agent can be expressed as a ratio. The inhibitor of FANCI and/or the FA pathway can be administered at a ratio of between about 100:1 to about 1:100, on a molar basis, in relation to the genotoxic anti-neoplastic agent, for example, at 1:100, 1:50, 1:10, 1:5, 1:2, 1:1, 2:1, 5:1, 10:1, 20:1, 50:1, or 100:1.

The genotoxic anti-neoplastic agent are agents which are used to treat neoplastic disorders, and include 1,3-Bis(2-Chloroethyl)-1-NitrosoUrea (BCNU), Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolomide, and Topotecan.

Dosages of the anti-neoplastic agents listed above have been well established for different types of neoplastic disorders. However, co-administration with inhibitors of the FA pathway can increase the sensitivity of the neoplastic disorders to the anti-neoplastic agents. Therefore, it is possible that the dosage of the anti-neoplastic agents will be less than is typically administered for the given neoplastic disorder. The lower dosage may have the additional advantage of reduced side effects. However, typically, the dosage of the anti-neoplastic agent is expected to be within about 20%-100% of the typical dosage for the given-neoplastic disorder, more typically between about 35%-100%.

In yet another embodiment, the present invention provides for a method of treating a neoplastic disorder in a subject in need thereof, comprising administering to the subject a combination of an effective amount of:

(a) an inhibitor of FANCI and/or the FA pathway or pharmaceutically acceptable salts, esters, derivatives, solvates or prodrugs thereof, and

(b) an inhibitor of a DNA damage repair pathway.

The inhibitor of a DNA damage repair pathway can be selected from the group consisting of PARP inhibitors, DNA-PK inhibitors, FA inhibitors, mTOR inhibitors, ERCC1 inhibitors, ERCC3 inhibitors, ERCC6 inhibitors, ATM inhibitors, XRCC4 inhibitors, Ku80 inhibitors, Ku70 inhibitors, XPA inhibitors, CHK1 inhibitors, CHK2 inhibitors, or pharmaceutically acceptable salts, esters, derivatives, solvates or prodrugs thereof.

In one embodiment, the non-FA DNA damage repair pathway is a pathway other than the FA pathway. In one embodiment, the inhibitor targets a pathway selected from the group consisting of the non-homologous end joining DNA damage repair pathway, the mismatch repair pathway, and the nucleotide excision pathway. In another embodiment, the inhibitor targets the non-homologous end joining DNA damage repair pathway. In yet another embodiment, the inhibitor targets the direct reversal pathway. In another embodiment, the inhibitor targets the mismatch repair pathway. In still another embodiment, the inhibitor targets the nucleotide excision repair pathway. In another embodiment, the inhibitor targets the base excision repair pathway.

Ideal dosages of the inhibitor of a DNA damage repair pathway, as described above for inhibitors of FANCI and/or the FA pathway, will depend upon the severity of the disease and the general state of the patient's own immune system, but generally range from 0.005 to 5.0 mg of the inhibitor per kilogram of body weight, with doses of 0.05 to 2.0 mg/kg/dose being more commonly used. Alternatively, the appropriate dosage can be determined empirically, inhibition of DNA damage repair pathways can be measured using biological samples taken from the subject. Therefore, an “effective dose” of an inhibitor of the DNA damage repair pathway can mean a dose required to reduce the level of the specific pathway, e.g., to about 70% or less when compared with a control sample, more typically to about 50% or less than a control sample. In this regard, a control sample is ideally taken from the same subject, before administration of the inhibitor.

In yet another embodiment, the present invention provides for a method of treating a neoplastic disorder in a subject in need thereof, comprising administering to said subject a combination of an effective amount of:

(a) an inhibitor of FANCI and/or the FA pathway or pharmaceutically acceptable salts, esters, derivatives, solvates or prodrugs thereof,

(b) an inhibitor of a non-FA DNA damage repair pathway, and

(c) a genotoxic anti-neoplastic agent or pharmaceutically acceptable salts, esters, derivatives, solvates or prodrugs thereof.

The inhibitor of FANCI and/or the FA pathway, its dosage and method of administration, are as described previously. Likewise, the inhibitor of a non-FA DNA damage repair pathway, as well as its dosage and method of administration are the same as previously described. However, as previously described, administration of inhibitors of the FA pathway, as well as of a non-FA DNA damage repair pathway, can heighten the sensitivity to a genotoxic anti-neoplastic agent. Therefore, it is possible that the dosage of the anti-neoplastic agents will be less than is typically administered for the given neoplastic disorder. The lower dosage may have the additional advantage of reduced side effects. However, typically, the dosage of the anti-neoplastic agent is expected to be within about 20%-100% of the typical dosage for the given neoplastic disorder, more typically between about 35%-100%.

The compounds of the present invention, or pharmaceutically acceptable salts, esters, derivatives, solvates or prodrugs thereof, can be formulated for oral, intravenous, intramuscular, subcutaneous, topical and/or parenteral administration for the therapeutic or prophylactic treatment of diseases. For oral or parental administration, compounds of the present invention can be mixed with conventional pharmaceutical carriers and excipients and used in the form of tablets, capsules, elixirs, suspensions, syrups, wafers and the like. The compositions comprising a compound of this present invention will contain from about 0.1% to about 99.9%, about 1% to about 98%, about 5% to about 95%, about 10% to about 80% or about 15% to about 60% by weight of the active compound.

The compounds of the present invention can be administered at separate times, using separate methods of administration. For example, in certain situations, it may be advantageous to administer the inhibitor of the FA pathway before, simultaneously with, or after administration of the genotoxic anti-neoplastic agent or other agents. Likewise, the method of administration of each compound will depend on the optimal means of administration thereof.

The pharmaceutical preparations disclosed herein are prepared in accordance with standard procedures and are administered at dosages that are selected to reduce, prevent, or eliminate cancer, or to provide a protective effect against genotoxic anti-neoplastic agents such as ionizing radiation. (See, e.g., Remington's Pharmaceutical Sciences, Mack Publishing Company, Easton, Pa.; and Goodman and Gilman, Pharmaceutical Basis of Therapeutics, Pergamon Press, New York, N.Y., the contents of which are incorporated herein by reference, for a general description of the methods for administering various antimicrobial agents for human therapy). The compositions of the present invention can be delivered using controlled (e.g., capsules) or sustained release delivery systems (e.g., biodegradable matrices). Examples of delayed release delivery systems for drug delivery suitable for administering compositions of the invention are described in U.S. Pat. Nos. 4,452,775, U.S. Pat. No. 5,239,660, and U.S. Pat. No. 3,854,480.

The pharmaceutically acceptable compositions of the present invention comprise one or more compounds of the present invention in association with one or more non-toxic, pharmaceutically acceptable carriers and/or diluents and/or adjuvants and/or excipients, collectively referred to herein as “carrier” materials, and if desired other active ingredients. The compositions may contain common carriers and excipients, such as corn starch or gelatin, lactose, sucrose, microcrystalline cellulose, kaolin, mannitol, dicalcium phosphate, sodium chloride and alginic acid. The compositions may contain crosarmellose sodium, microcrystalline cellulose, sodium starch glycolate and alginic acid.

Tablet binders that can be included are acacia, methylcellulose, sodium carboxymethylcellulose, polyvinylpyrrolidone (Providone), hydroxypropyl methylcellulose, sucrose, starch and ethylcellulose.

Lubricants that can be used include magnesium stearate or other metallic. stearates, stearic acid, silicon fluid, talc, waxes, oils and colloidal silica.

Flavoring agents such as peppermint, oil of wintergreen, cherry flavoring or the like can also be used. It may also be desirable to add a coloring agent to make the dosage form more aesthetic in appearance or to help identify the product comprising a compound of the present invention.

For oral use, solid formulations such as tablets and capsules are particularly useful. Sustained released or enterically coated preparations may also be devised. For pediatric and geriatric applications, suspension, syrups and chewable tablets are especially suitable. For oral administration, the pharmaceutical compositions are in the form of, for example, a tablet, capsule, suspension or liquid. The pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica or talc: disintegrants, for example, potato starch, flavoring or coloring agents, or acceptable wetting agents. Oral liquid preparations generally are in the form of aqueous or oily solutions, suspensions, emulsions, syrups or elixirs and may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For intravenous (iv) use, compounds of the present invention can be dissolved or suspended in any of the commonly used intravenous fluids and administered by infusion. Intravenous fluids include, without limitation, physiological saline or Ringer's solution.

Formulations for parental administration can be in the form of aqueous or non-aqueous isotonic sterile injection solutions or suspensions. These solutions or suspensions can be prepared from sterile powders or granules having one or more of the carriers mentioned for use in the formulations for oral administration. The compounds can be dissolved in polyethylene glycol, propylene glycol, ethanol, corn oil, benzyl alcohol, sodium chloride, and/or various buffers.

For intramuscular preparations, a sterile formulation of compounds of the present invention or suitable soluble salts forming the compound, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g. an ester of a long chain fatty acid such as ethyl oleate.

For topical use the compounds of present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the compounds of the present invention can be presented in liquid or semi-liquid form formulated in hydrophobic or hydrophilic bases as ointments, creams, lotions, paints or powders.

For rectal administration the compounds of the present invention can be administered in the form of suppositories admixed with conventional carriers such as cocoa butter, wax or other glyceride.

Alternatively, the compound of the present invention can be in powder form for reconstitution in the appropriate pharmaceutically acceptable carrier at the time of delivery. In another embodiment, the unit dosage form of the compound can be a solution of the compound or a salt thereof in a suitable diluent in sterile, hermetically sealed ampoules.

The amount of the compound of the present invention in a unit dosage comprises a therapeutically-effective amount of at least one active compound of the present invention which may vary depending on the recipient subject, route and frequency of administration. A subject refers to an animal such as an ovine or a mammal, including a human.

According to this aspect of the present invention, the novel compositions disclosed herein are placed in a pharmaceutically acceptable carrier and are delivered to a recipient subject (including a human subject) in accordance with known methods of drug delivery. In general, the methods of the invention for delivering the compositions of the invention in vivo utilize art-recognized protocols for delivering the agent with the only substantial procedural modification being the substitution of the compounds of the present invention for the drugs in the art-recognized protocols.

The compounds of the present invention provide a method for treating pre-cancerous or cancerous conditions, or for use as a protective agent against genotoxic anti-neoplastic agents. As used herein, the term “unit dosage” refers to a quantity of a therapeutically effective amount of a compound of the present invention that elicits a desired therapeutic response. The term “treating” is defined as administering, to a subject, a therapeutically effective amount of at least one compound of the present invention, both to prevent the occurrence of a pre-cancer or cancer condition, or to control or eliminate pre-cancer or cancer condition. The term “desired therapeutic response” refers to treating a recipient subject with a compound of the present invention such that a pre-cancer or cancer condition is reversed, arrested or prevented in a recipient subject.

The compounds of the present invention can be administered as a single daily dose or in multiple doses per day. The treatment regime may require administration over extended periods of time, e.g., for several days or for from two to four weeks. The amount per administered dose or the total amount administered will depend on such factors as the nature and severity of the disease condition, the age and general health of the recipient subject, the tolerance of the recipient subject to the compound and the type of cancer, the sensitivity of the cancer to therapeutic agents, and, if used in combination with other therapeutic agent(s), the dose and type of therapeutic agent(s) used.

A compound according to this invention may also be administered in the diet or feed of a patient or animal. The diet for animals can be normal foodstuffs to which the compound can be added or it can be added to a premix.

The compounds of the present invention may be taken in combination, together or separately with any known clinically approved agent to treat a recipient subject in need of such treatment.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Methods

### Cell Lines

Complemented cell lines PD20 and GM6914 were described previously (Taniguchi et al. (2002) Cell 109: 459-472), DR-U2OS were provided by Maria Jasin (Xia et al. (2006) Mol Cell 22: 719-729). GM02188 was obtained from Coriell, BD0952 from European Collection of Cell Cultures (http://www.ecacc.org.uk), and U20S from American Cell Culture Collection (ATCC). The adherent cell lines were grown in Dulbecco Modified Eagle medium (DMEM) supplemented with 100 units of penicillin per ml, 0.1 mg streptomycin per ml, L-glutamine (2 mM), non-essential amino acids (0.1 mM), and 10% or 15% (v/v) FBS (Invitrogen) depending on the cell line, and lymphoblastoid lines were grown in RPMI with the same supplementation. Retroviral transduction of the lymphocytes was performed by spirning 1×10⁶ with a freshly-collected virus supplemented with 8 μg of polybrene per ml of supernatant at 2500 rpm for 45 minutes at room temperature.

### Antibodies

Antibodies were as follows: KIAA1794; BL999 and BL1000 (Bethyl), rabbit FANCD2 (Novus), mouse FANCD2 (Santa Cruz), FANCA (Rockland), ORC2 (BD Bioscience), Vinculin (Sigma), HA (Covance), MYC (Covance), SMC3pS1083 (Bethyl), PhosphoH3 (Upstate), Ran (BD Bioscience), γH2AX (Upstate). For the EPs, anti-HA affinity matrix (Roche), anti-FLAG M2 agarose (Sigma), c-MYC (Santa Cruz) and Protein A/G PLUS-Agarose (Santa Cruz) were used. Secondary antibodies for IF were from Molecular Probes and Amersham and for western blots were from Jackson Laboratories.

### FANCI Cloning

PCR was performed using Platinum Taq DNA Polymerase High Fidelity (Invitrogen) on a human cDNA library (Elledge et al. (1991) Proc Natl Acad Sci USA 88: 1731-1735). The total RNA from BD0952 cells was isolated using Trizol (Invitrogen). The RNA was reverse transcribed with Superscript III (Invitrogen) and dT primers. The PCR step was performed using Platinum Pfx DNA Polymerase (Invitrogen). Genomic DNA was prepared using DNeasy Tissue kit (Qiagen). Primers used for KIAA1794 cDNA cloning were 5′-CCGCTCGAGGACCAGAAGATTTTATCTCTAGCAG-3′ (SEQ ID NO: 1) and 5′-CCGGTTAACTTAACTCAGGCATTTCATTTATTTT-3′ (SEQ ID NO: 2). The same primers were also used for cloning the cDNA from BD0952 (FANC I) cells. The genomic PCR primers were: 1st coding exon: 5′-TTCAGGATTATTTTGGTTAGGTTA-3′ (SEQ ID NO: 3) and 5′-GGTCACAAATGCCCTCAAG-3′ (SEQ ID NO: 4) 3rd coding exon: 5′-TCAAAGCCCTTAACCATTGC-3′ (SEQ ID NO: 5) and 5′-TGCCATCTTACCTCCAGCAT-3′ (SEQ ID NO: 6) 36^{th} coding exon: 5′-TCTTGATCTGATGACCTGAACC-3′ (SEQ ID NO: 7) and 5′-GTCGGGGCAACTTCATAGGAT-3′ (SEQ ID NO: 8).

### Mutagenesis

The QuikChange® II XL Site-Directed Mutagenesis Kit (Stratagene) or QuikChange® Multi Site-Directed Mutagenesis Kit (Stratagene) was used to make mutation(s) in FANCI. The K523R mutation was generated using the QuikChange® II XL Site-Directed Mutagenesis Kit with mutagenic primers

(SEQ ID NO: 9)

5′-GCTTGATACTTGTCCTTCGGCGAGCTATGTTTGCCAACCAGC-3′
and

(SEQ ID NO: 10)

5′-GCTGGTTGGCAAACATAGCTCGCCGAAGGACAAGTATCAAGC-3′.

The QuikChange® Multi Site-Directed Mutagenesis Kit was used to make the P55L mutation (primer 5′-CTTCAAAGGTTCCCTCTGCTCTGAGGAAGCTGG-3′ (SEQ ID NO: 11)) and the R1285Q mutation (5′-GCTCAGCACCTCACAAGACTTCAAGATCAAAGG-3′ (SEQ ID NO: 12)) in FANCI.

siRNAs

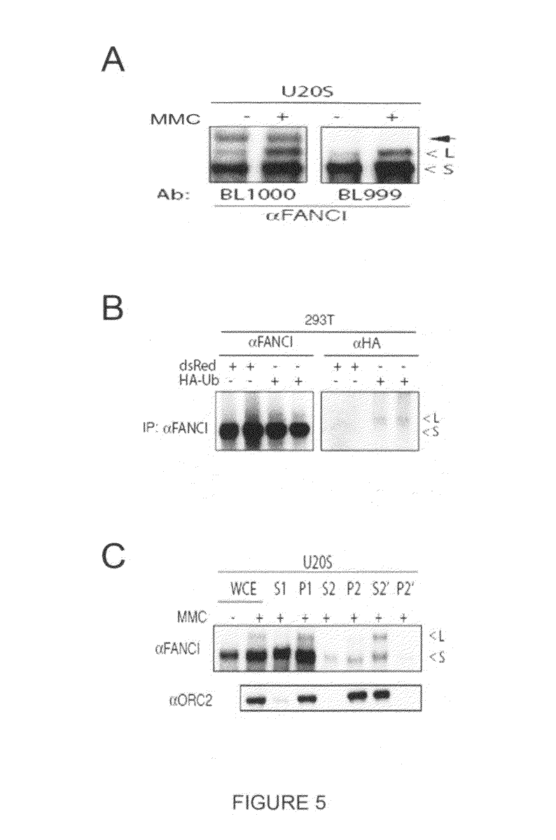

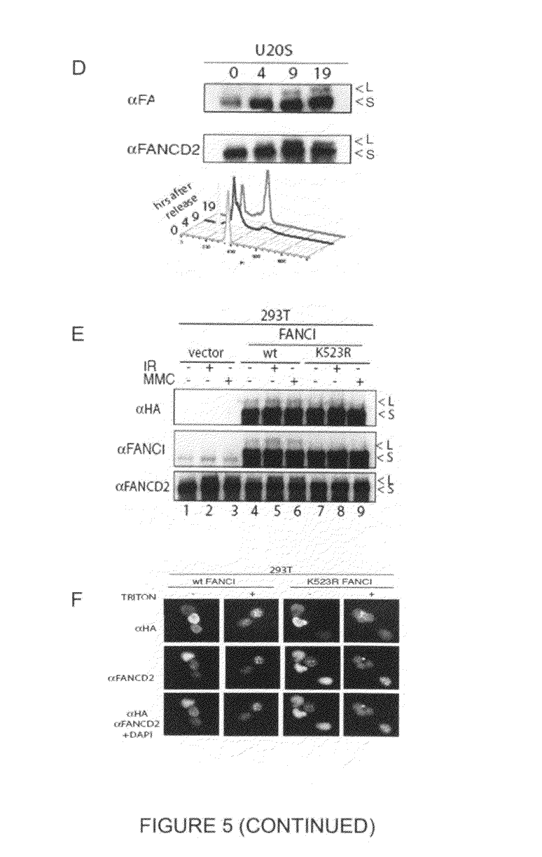

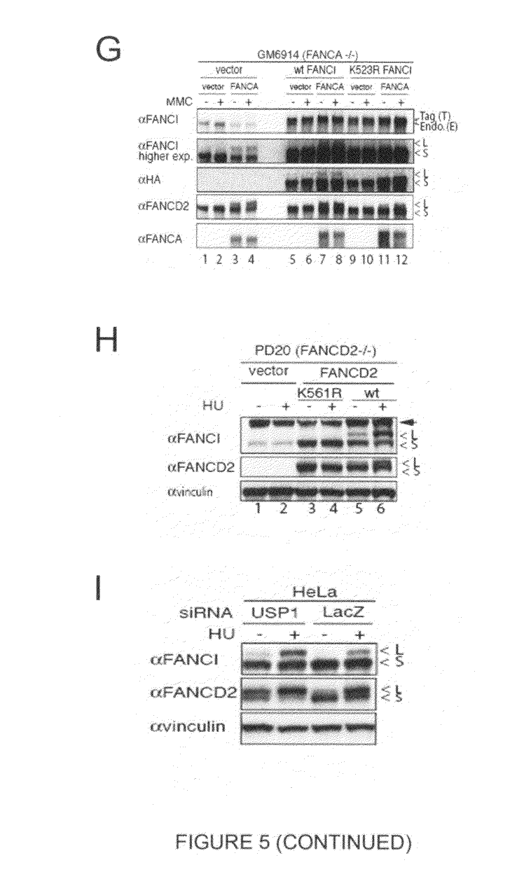

Stealth siRNAs (Invitrogen) were transfected using Oligofectamine (Invitrogen) at final concentration of 85 nM total siRNAs. Assays were done 48-72 hours after transfection. Unless indicated otherwise, a combination of three siRNAs against the same gene were used. Target sequences were as follows. siRNAs were purchased from Invitrogen unless otherwise stated (siRNAs used in the experiments shown in FIGS. 1D and 5I were purchased from Qiagen):

lacZ (Qiagen)

(SEQ ID NO: 13)

5′-AACGTACGCGGAATACTTCGA-3′
FANCI (Qiagen)

(SEQ ID NO: 14)

5′-CTGGCTAATCACCAAGCTTAA-3′
USP1 (Qiagen)

(SEQ ID NO: 15)

5′-TCGGCAATACTTGCTATCTTA-3′
ATM:

(SEQ ID NO: 16)

5′-GCGCAGTGTAGCTACTTCTTCTATT-3′,

(SEQ ID NO: 17)

5′-GGGCCTTTGTTCTTCGAGACGTTAT-3′,

(SEQ ID NO: 18)

5′-GCAACATTTGCCTATATCAGCAATT-3′
ATR:

(SEQ ID NO: 19)

5′-GGGAAATAGTAGAACCTCATCTAAA-3′,

(SEQ ID NO: 20)

5′-GGTCTGGAGTAAAGAAGCCAATTTA-3′,

(SEQ ID NO: 21)

5′-CCACCTGAGGGTAAGAACATGTTAA-3′
FANCI #1:

(SEQ ID NO: 22)

5′-TCTCCTCAGTTTGTGCAGATGTTAT-3′
FANCI #2:

(SEQ ID NO: 23)

5′-GGCAGCTGTGTGGACACCTTGTTAA-3′
FANCI #3:

(SEQ ID NO: 34)

5′-GCTGGTGAAGCTGTCTGGTTCTCAT-3′
FANCD2 #2:

(SEQ ID NO: 25)

5′-TTAGTTGACTGACAATGAGTCGAGG-3′
FANCD2 #3:

(SEQ ID NO: 26)

5′-AATAGACGACAACTTATCCATCACC-3′
BRCA1:

(SEQ ID NO: 27)

5′-AAATGTCACTCTGAGAGGATAGCCC-3′,

(SEQ ID NO: 28)

5′-TTCTAACACAGCTTCTAGTTCAGCC-3′,

(SEQ ID NO: 29)

5′-TAGAGTGCTACACTGTCCAACACCC-3′
FANCA:

(SEQ ID NO: 30)

5′-GGAAGATATCCTGGCTGGCACTCTT-3′,

(SEQ ID NO: 31)

5′-CCAGCATATTCAGGAGGCCTTACTA-3′,

(SEQ ID NO: 32)

5′-TCCCTCCTCACAGACTACATCTCAT-3′

In these experiments, cells were transfected at a concentration of 20 nM using Hyperfect according to manufacturer's instructions.

### Immunofluorescence

Cells grown on autoclaved cover slips were processed were rinsed with phosphate-buffered-saline (PBS) and fixed in 3.7% (w/v) formaldehyde (Sigma) diluted in PBS for 10 minutes at room temperature. Cells were washed once with PBS, permeabilized in 0.5% (v/v) NP40 in PBS for 10 minutes, washed again in PBS, and blocked with PBG (0.2% [w/v] cold fish gelatin, 0.5% [w/v] BSA in PBS) for 20 minutes. Coverslips were incubated for 2 hours at room temperature or at 4° C. overnight in a humidified chamber with a primary antibody and after washing 3 times for 5 minutes in PBG, then were incubated with the appropriate secondary antibody. After three additional washes in PBG, the coverslips were embedded in Vectashield (Vector Laboratories) supplemented with DAPI. Triton pre-extraction was performed by incubating cells for 5 minutes at room temperature with 0.5% Triton in PBS. After gentle rinse with PBS, cells were fixed and processed as above. Images were captured with Axioplan2 Zeiss microscope with a AxioCam Mrm Zeiss digital camera supported by Axovision 4.5 software. For the IF on lymphoblastic cell lines the coverslips were treated with sterile Poly-D-lysine hydrobromide, molecular weight >300,000 (Sigma), as suggested by the manufacturer. After the cells attached (several hours), the coverslips were processed as indicated above. Any co-staining experiments included proper controls to exclude crossing of signal between different channels.

### Chromatin Fractionation and Immunoprecipitations

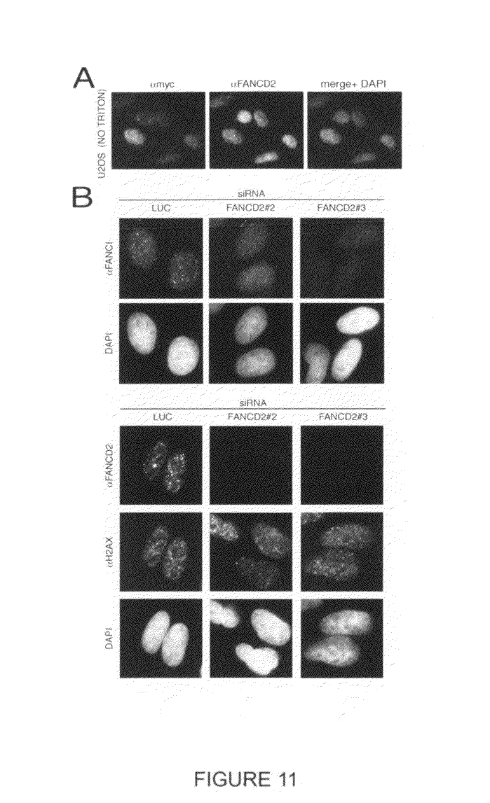

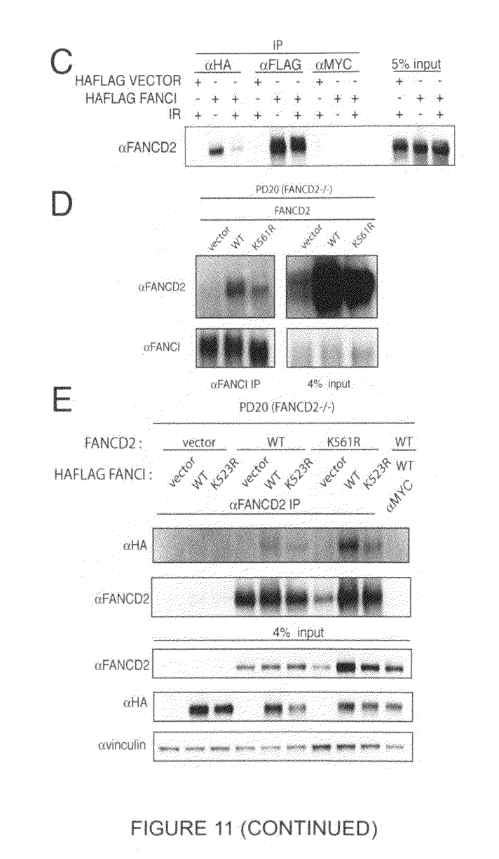

Chromatin fractionation was performed as described (Mendez and Stilhman (2000) Mol Cell Biol 20: 8602-8612; Zou et al. (2002) Genes Dev 16: 198-208). For immuoprecipitations, cells were lysed in TBS (20 mM Tris +150 mM NaCl) supplemented with 0.5% NP-40, protease Inhibitors (Roche), 1 mM PMSF, 5 mM NaF, and 5 mM Na3VO4 and 50U of Benzonase (Novagen) per ml of lysis buffer. The experiment shown in FIG. 11C was performed without addition of Benzonase. 1 mg protein extract was incubated with 2 μg of the indicated antibody and 5 μl of Protein A/G PLUS-Agarose (Santa Cruz). Following three washes in lysis buffer, the immunoprecipitates were eluted in tris-Glycine SDS sample buffer and size-fractionated on a Tris-Glycine gel (Invitrogen). Streptavidin immunoprecipitation under denaturating conditions was performed as described (Tagwerker et al., 2006) except the His-purification step was omitted. Streptavidin sepharose (GE Healthcare) was used with lysis and wash buffer consisting of 8 M urea, 200 mM NaCl, 100 mM Tris pH 8, 0.5% SDS, 0.5% NP40.

### Multicolor Competition Assay

U2OS cells were infected with MSCVgfp or MSCVdsRed, which were packaged in 293T by co-transfection with VSVG vector using TransIT-293 (Mirus). Without selection, the cells were sorted using the Aria Sorter (BD) for intermediate expression. The gfp cells grew slightly faster than the rfp cells and this was taken into account when calculating the changes in survival due to treatment with DNA damaging agents. siRNA transfections were performed as described above with gfp cells being transfected with a control siRNA (luciferase) and rfp cells with an siRNA of interest. On the third day after transfections, gfp and rfp cells were counted and mixed in Ito 1 ratio and were left untreated or were treated with IR or MMC. The concentration of Mitomycin C (Sigma) was chosen to result in about 50% survival of non-transfected cells, which was about 70 nM MMC for U2OS cells. After 7 days of culture, all cells were collected and analyzed using Cytomix FC500 Analyzer (Beckman Coulter). Relative survival of Luc siRNA-treated cells after damage was set to 100%.

### G2/M Checkpoint Assay

U2OS cells were transfected with individual siRNAs for three days in a 96 well format. Cells were irradiated with 5 Gy and allowed to recover for 1 hr before the addition of 100 ng nocodazole per ml of media to trap cells that bypass the G2/M checkpoint. Cells were fixed and stained with an antibody against Phospho-H3 9 hours after irradiation. Plates were imaged on an automated ImageXpress Micro (Molecular Dynamics) at 10× and the mitotic index was calculated using the MetaExpress Software package. An average of 1000 cells was counted per well. Wells scoring above control levels were visually inspected to verify accurate scoring by the software.

### Radioresistant DNA Synthesis Assay

RDS assays to evaluate the intra-S phase checkpoint were done as described previously (Silverman et al. (2004) Genes Dev 18: 2108-2119). Briefly, U20S cells were transfected with control siRNA or siRNAs against KIAA1794 (combination of 3 siRNAs, approximately 30 nM of each) using oligofectamine (Invitrogen). 24 hours later, medium containing 10 nCi/mL of [methyl-14C] thymidine (Amersham, CFA532) was added and cells were incubated for 24 hours. Then, medium without label was added for 24 hours. The cells were then irradiated (Cesium 137 source) with 5-15 Gy. Following a 30-minute incubation at 37 degrees, the cells were pulse labeled with 2.5 uCi/mL [methyl-3H] thymidine (Amersham, TRK758) for 20 minutes and then washed twice with medium containing 2.5 mM cold thymidine (no serum). Cells were harvested by trypsinization and TCA precipitation was performed on Whatman glass microfibre filters (GF/C, 25 mM, Fisher) using a vacuum manifold. Following an ethanol wash, the filters were dried and counted using a liquid scintillation counter (Beckman LS6000). The ratio of ³H counts per minute to ¹⁴C counts per minute, corrected for those counts per minute that were the result of channel crossover, were a measure of DNA synthesis.

### Homologous Recombination Assay

HR assay was performed as described (Nakanishi et al. (2005) Proc Natl Acad Sci USA 102: 1110-1115; Xia et al. (2006) Mol Cell 22: 719-729), except instead of transfecting cells with an I-SceI expressing plasmid, an adenovirus AdNGUS24i (provided by Frank Graham, McMaster University) expressing the I-SceI enzyme was used. Control adenovirus AdCA36 (Addison et al. (1997) J Gen Virol 78: 1653-1661) expressed β-galactosidase. Five or 10 pfu of adenovirus per cell was used since this level of virus resulted in 100% infection but had no visible deleterious effects on cells. Events were gated to exclude any doublets. Both gated and non-gated analysis gave similar results.

### Cell Cycle Synchronization

U2OS cells were treated with 2.5 mM thymidine for 24 hours, washed three times and released into 100 ng nocodazole per ml of media, incubated for 12 hours and collected by mitotic shakeoff. Cells were washed three times, counted and plated for collection at different times. For cell cycle analysis, collected cells were resuspended in 100 μl (PBS). While vortexing, 2 ml of ice cold 70% (v/v) ethanol were added drop-wise and the suspension was stored at 4° C. at least overnight. 30 min before FACS, cells were spun down, resuspended in propidium iodine (PI) mix (500 μl PBS, 10 μl RNase [of stock solution of 10 mg/ml], 25 μl PI [of stock solution of 1 mg/ml]), and analyzed using LSR2 (Becton Dickinson). Cell cycle analysis was performed using FlowJo.

### Mitomycin C Sensitivity Assay

Logarithmically growing cells were counted and diluted to 2×10⁵ cells per ml, plated in triplicate for each drug dose and treated with different concentrations of freshly made Mitomycin C. After 6 days in culture, cells were harvested and counted using a Z2 Coulter Couhter (Beckman Coulter). Cell numbers in the samples treated with the drug were normalized to the cell numbers in the untreated sample.

### Bioinformatics

BLAST was used for homology searches (http://www.ncbi.nlm.nih.gov/BLAST/; Altschul et al. (1997) Nucleic Acids Res 25: 3389-3402). The SCOP database can be found at http://scop.mrc-lmb.cam.ac.uk/scop/ (Murzin et al. (1995) J Mol Biol 247: 536-540). Alignments were performed in ClustalX and were rendered using ESPript 2.2 (http://espript.ibcp.fr; Gouet et al. (1999) Bioinformatics 15: 305-308). The GenBank accession number for FANCI is EF469766.

### Antibodies to the FANCI Protein

Rabbit polyclonal antisera to the human FANCI protein are available commercially from Bethyl and Abcam.

In addition, rabbit polyclonal and mouse monoclonal antibodies are generated to FANCI using (1) full-length human FANCI protein that has been synthesized in insect (SF9) cells and injected into rabbits and mice, for generation of polyclonal and murine monoclonal antibodies, respectively and (2) a GST-FANCI fusion protein, containing the N-terminal 200 amino acids of FANCI fused to GST, that has been generated for use as antigen.

### Example 2

### KIAA1794/FANCI was Identified as a Phosphoprotein

KLAA1794/FANCI was identified as a protein whose phosphorylation was induced upon IR treatment (Matsuoka et al., submitted). In that study, SILAC (reviewed in (Mann (2006) Nat Rev Mol Cell Biol 7: 952-958)) and peptide immunoprecipitation (Rush et al. (2005) Nat Biotechnol 23: 94-101) using phosphospecific antibodies followed by mass spectrometry before and after DNA damage was used to identify those proteins that were inducibly phosphorylated on SQ or TQ motifs. Three phosphorylation sites were detected in a human KIAA1794 protein: S730, T952, S1121, and two other sites in the mouse protein S555, T558. The KIAA1794 protein was renamed FANCI, since, as shown below, the locus encoding this protein was identified as mutated in an individual with Fanconi anemia complementation group I. Immunoblotting of FANCI after IR with a phospho-SQ antibody confirmed its inducible phosphorylation (refer to FIG. 1A, showing Western analysis with an antibody raised against a phosphorylated form of SMC3 (SMC3 pS1083) on immunoprecipitates performed with FANCI antibody (BL999) from 293T extracts before and after DNA damage), thus placing it in the ATM/ATR pathway.

### Example 3

### Multicolor Competition Assay (MCA) Used to Study DNA Damage Sensitivity

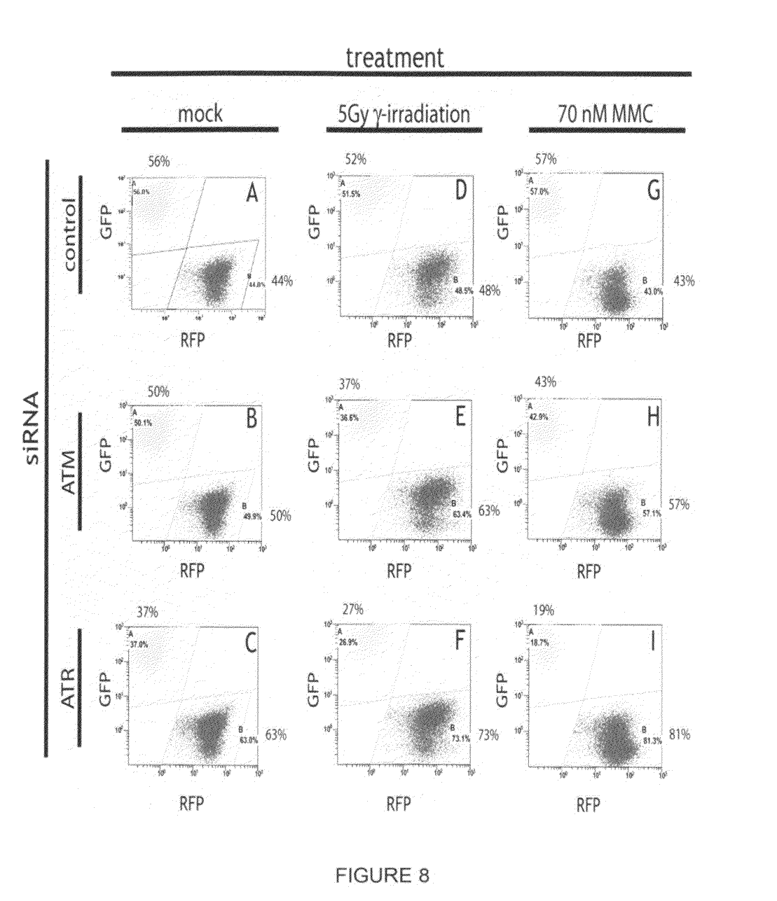

To efficiently study DNA damage sensitivity of cells with a variety of genetic perturbations, a simple competition assay was developed that proved both quantitative and fast (refer to FIG. 1B, which schmatically illustrates the multi-color competition assay (MCA)—here, the knockdown of a protein of interest caused the gfp cells to become DNA damage sensitive without influencing their proliferative capacity in the absence of damage. The relative resistance to damage of the si-treated cells was 40% of the non-si treated cells). Two populations of U20S (osteosarcoma) cells differing only in their color were created by expression of red fluorescent protein (RFP) or green fluorescent protein (GFP). siRNA depletion of the protein of interest was carried out in the green cells while the red cells were transfected with control siRNA. Equal numbers of green and red cells were mixed, left untreated or treated with gamma-irradiation or mitomycin C (MMC). After 7 days, cells were harvested and a ratio of red to green cells was determined using flow cytometry. The green to red ratio in untreated cells acted as a control for the relative cell growth. The assay was validated using siRNAs targeting ATM (IR-sensitivity) and ATR (MMC- and IR-sensitivity; refer to FIGS. 1C and 8.

FIG. 1C presents the results of MCA analysis in U2OS cells treated with siRNAs against ATM and ATR and three different siRNAs against FANCI, while FIG. 8 shows raw data from the multicolor competition assay performed with cells that were depleted of ATM or ATR).

MCA was applied to study a subset of ATM and ATR substrates (Matsuoka et al., submitted). Cells treated with a combination of three siRNAs against one of the tested proteins, FANCI (KIAA1794 a.k.a. FLJ10719), demonstrated 60% survival after 70 nM MMC treatment and 91% of survival after 3 Gy IR treatment relative to control siRNA transfected cells (data not shown). To exclude off target effects, three siRNAs were tested independently. Two of three siRNAs reproduced the phenotype of MMC-sensitivity with only a slight effect on the IR sensitivity (refer to FIG. 1C). This decreased survival was due to a DNA repair defect, as metaphase spreads of primary fibroblasts transfected with FANCI siRNA and treated with MMC revealed frequent cytogenetic abnormalities including chromatid and chromosome breaks as well as radial forms (refer to FIG. 1D, which displays cytogenetic abnormalities in IMR90 cells transfected with siRNA against KIAA1794 or LacZ control and treated with 0.5, or 7.5 ng MMC per ml; an asterisk in FIG. 1D indicates a statistically significant difference in means as calculated by the t-test; the experiment with 7.5 ng MMC per ml was performed once), hallmarks of Fanconi anemia.

### Example 4

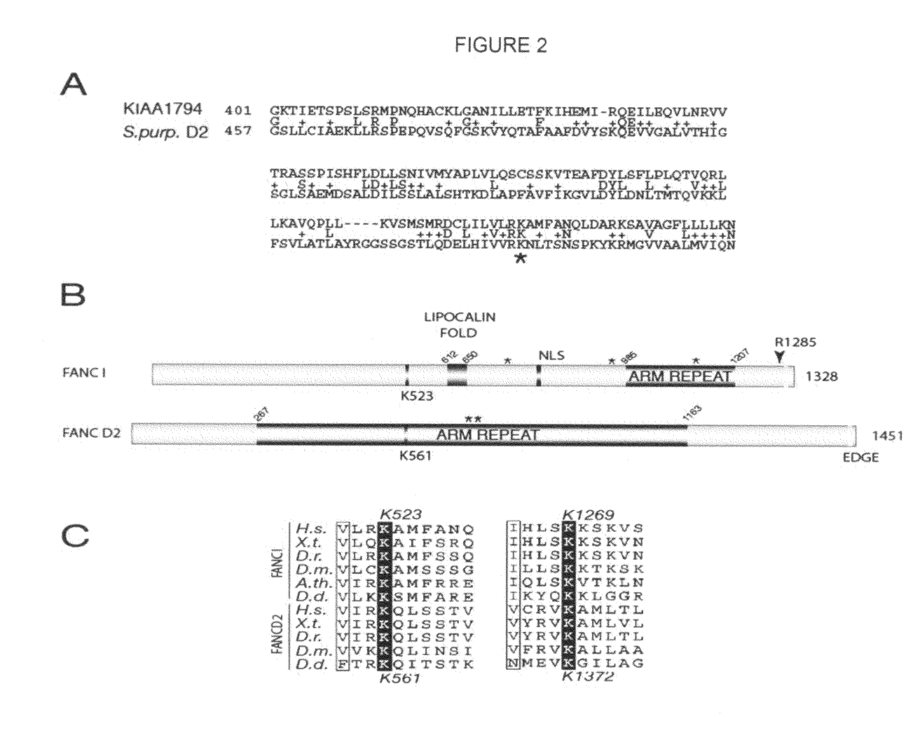

### FANCI was Identified as Homologous to FANCD2 BLAST analysis with FANCI revealed high conservation among eukaryotes from human to Dictyostelium but not yeasts and limited conservation to a predicted partial S. purpuratus sequence similar to FANCD2 (refer to FIG. 2A, showing a BLAST alignment identifying human KIAA1794 conservation with a portion of the Strongylocentrotus purpuratus (S.p.) ortholog of FANCD2. A star in FIG. 2A indicates the lysine corresponding to K561 in FANCD2). The homology region extended over 151 amino acids with 19% identity, 45% similarity. The coding region of FANCI was amplified from a human lymphocyte cDNA library (Elledge et al. (1991) Proc Natl Acad Sci USA 88: 1731-1735) and recovered an open reading frame of 3984 nucleotides, coding for a 1328 AA protein of a calculated molecular weight 150 kDa. This cDNA corresponded to a putative splice variant isoform 3 of the KLAA1794 (Q9NVI1) locus on chromosome 15q25-q26.

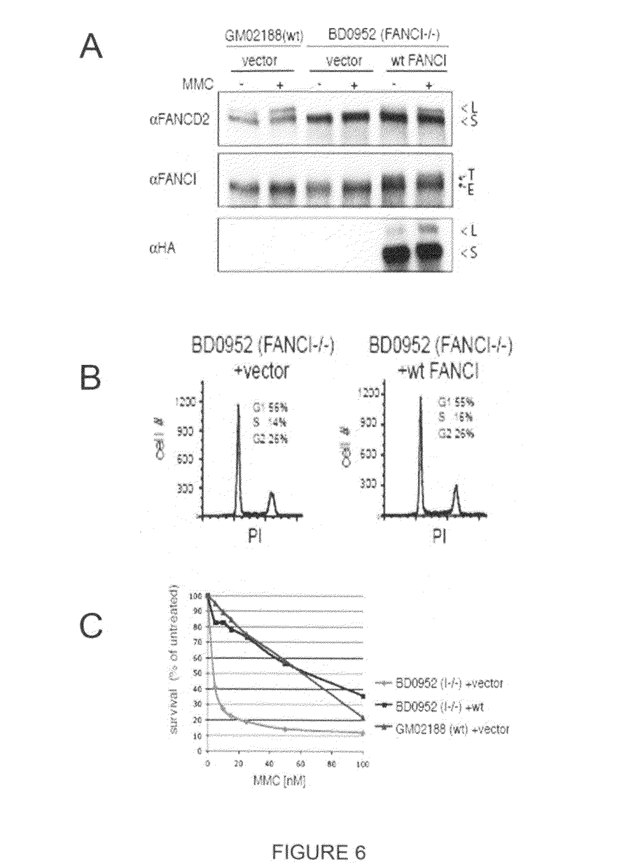

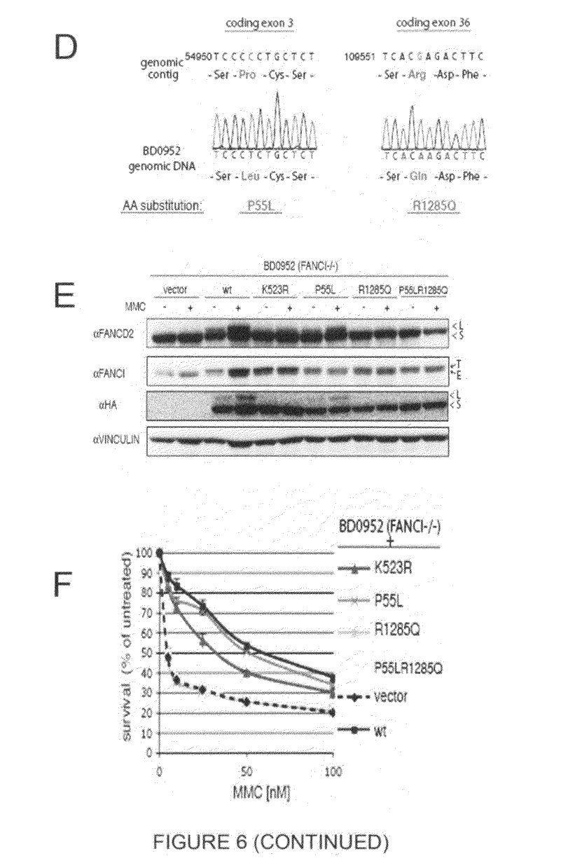

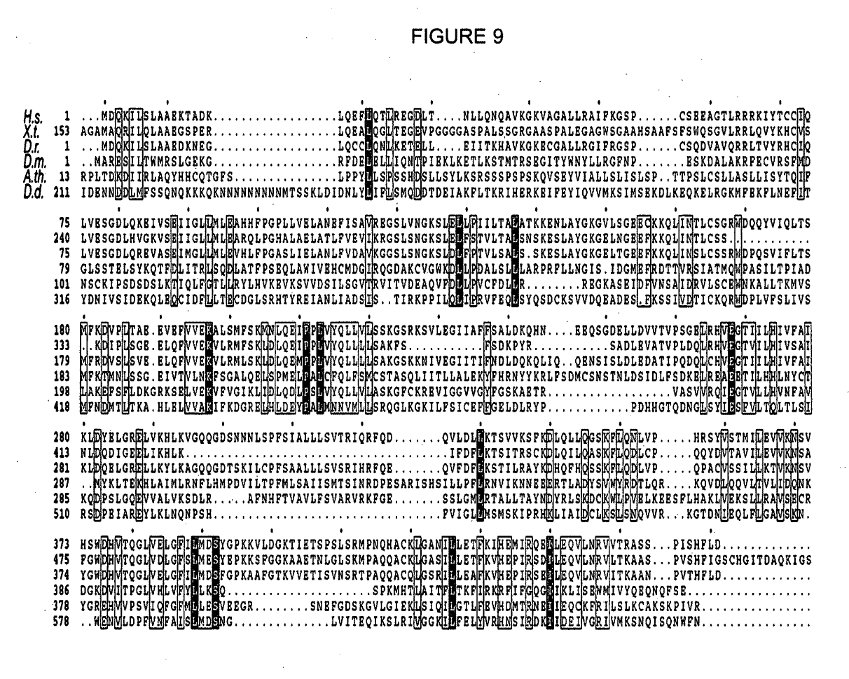

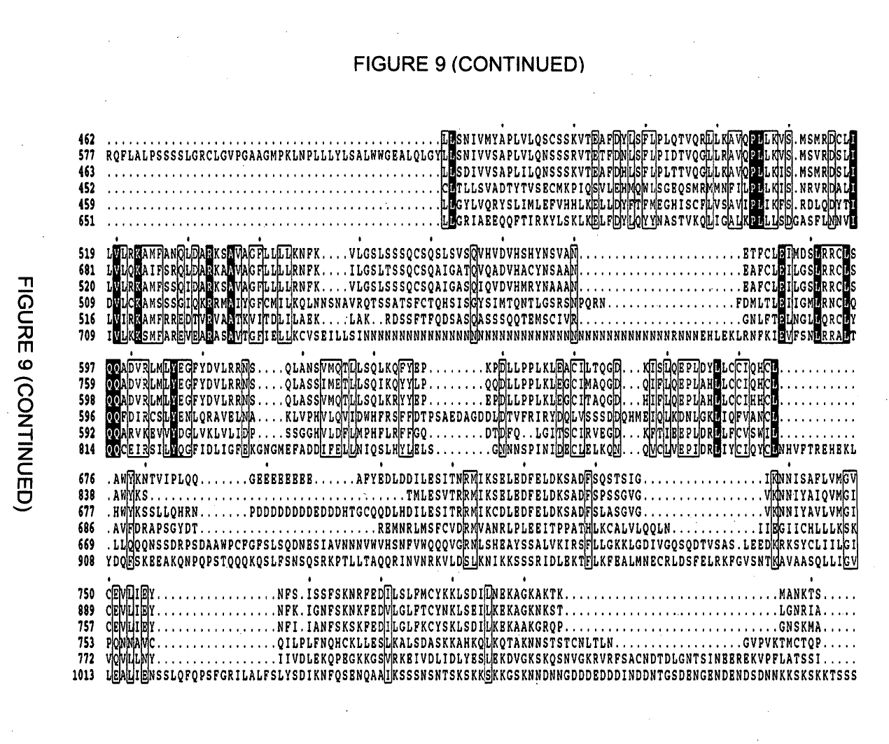

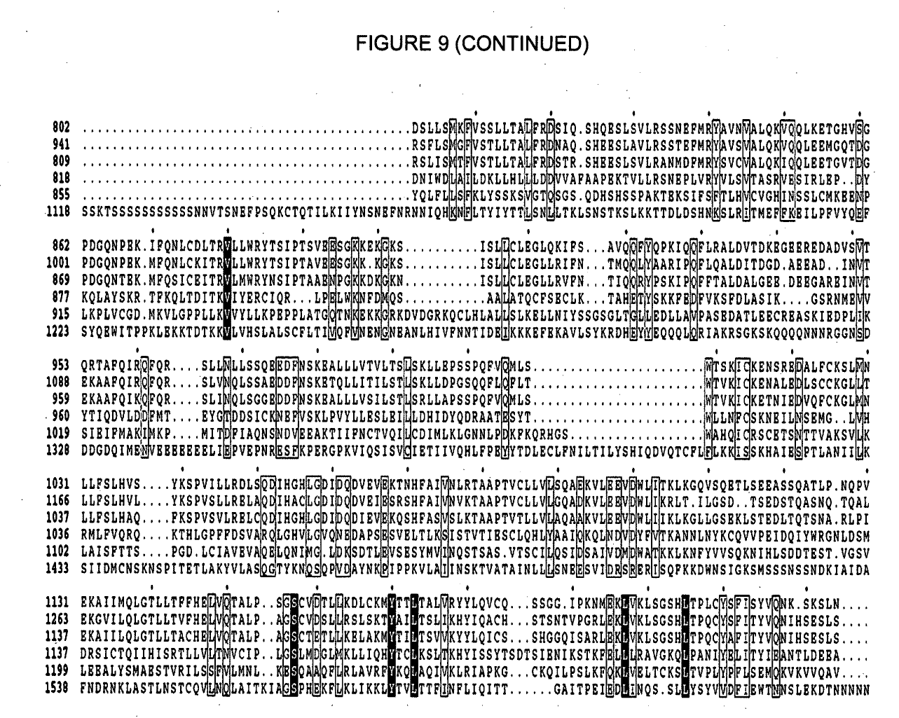

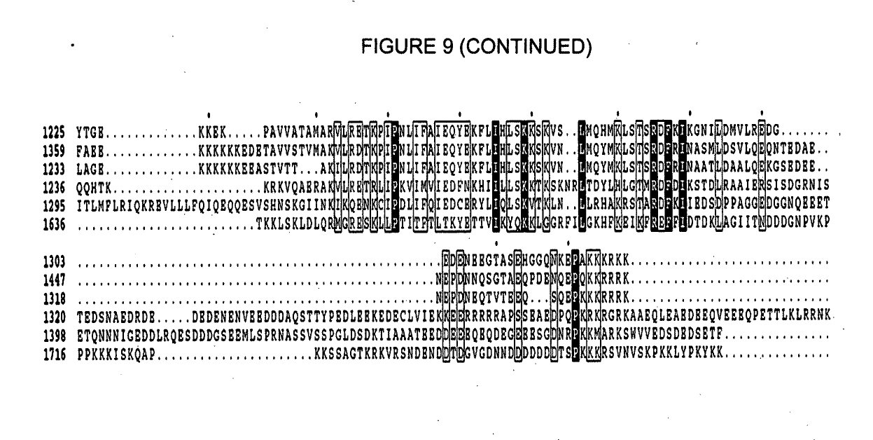

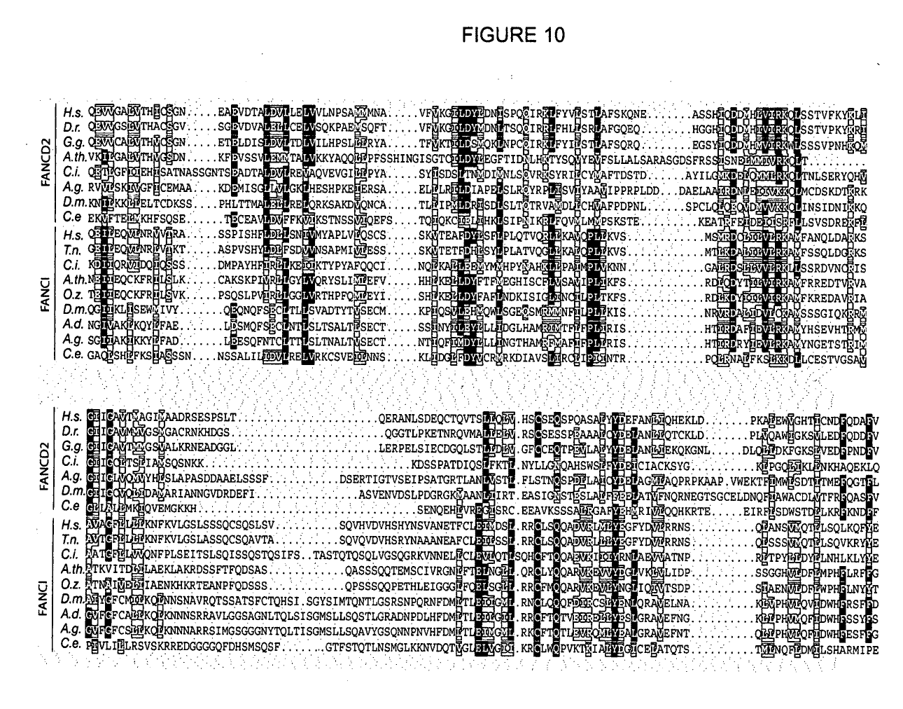

Alignment of FANCI and FANCD2 revealed a modest 13% identity and 20% similarity across the entire protein (refer to FIGS. 2B and 10. FIG. 2B presents an alignment of FANCI and FANCD2 that identified a conserved lysine K523, while FIG. 10 shows an alignment of FANCD2 and FANCI from Homo sapiens (H.s.), Danio rerio (D.r.), Gallus gallus (G.g.), Arabidopsis thaliana (A.th.), Ciona intestinalis (C.i.), Anopheles gambiae (A.g.), Drosophila melanogaster (D. m.), Caenorhabditis elegans (C.e.), Tetraodon nigroviridis (T.n.), Oryza sativa (O.z.), and Aedes aegypti (A.d.)). (It is noted that an alignment of FANCI sequence from Homo sapiens (H.s.), Xenopus tropicalis (X.t.), Danio rerio (D.r.), Drosophila melanogaster (D.m.), Arabidopsis thaliana (A.th.), and Dictyostelium discoideum (D.d.), with identities highlighted, is also presented in FIG. 9.) Comparable levels of similarity were found between the FANCD2 and FANCI paralogs in other species including A. thaliana, and D. melanogaster. The most striking conservation between FANCI and FANCD2 across the species surrounded the site that had been previously shown to be monoubiquitinated in FANCD2 and to be essential for the functionality of the FA pathway, K523 in FANCI and K561 in FANCD2 (refer to FIGS. 2B and 2C; Garcia-Higuera et al. (2001) Mol Cell 7: 249-262; FIG. 2C shows a schematic cross-species alignment of FANCI and FANCD2. Highlighted within FIG. 2C are two regions predicted by the SCOP database (Murzin et al. (1995) J Mol Biol 247: 536-540) as ARM repeats which represent alpha-alpha superhelix folds (aa 985-1207 in FANCI and aa 267-1163 in FANCD2) and a lipocalin fold (aa 612-650), which is predicted to bind hydrophobic ligands in its interior. Also shown is putative bipartite NLS (aa 779-795) identified in FANCI. Light stars indicate phosphorylation sites identified in human or mouse proteins (Matsuoka et al., submitted). Dark stars indicate the ATR sites in FAND2. The EDGE sequence was also identified to be conserved between the proteins. An arrowhead indicates the disease-causing mutation in a cell line of Fanconi anemia complementation group I (refer to FIG. 6)).

### Example 5

### Role of FANCI in Cell Cycle Checkpoints and DNA Repair Pathways

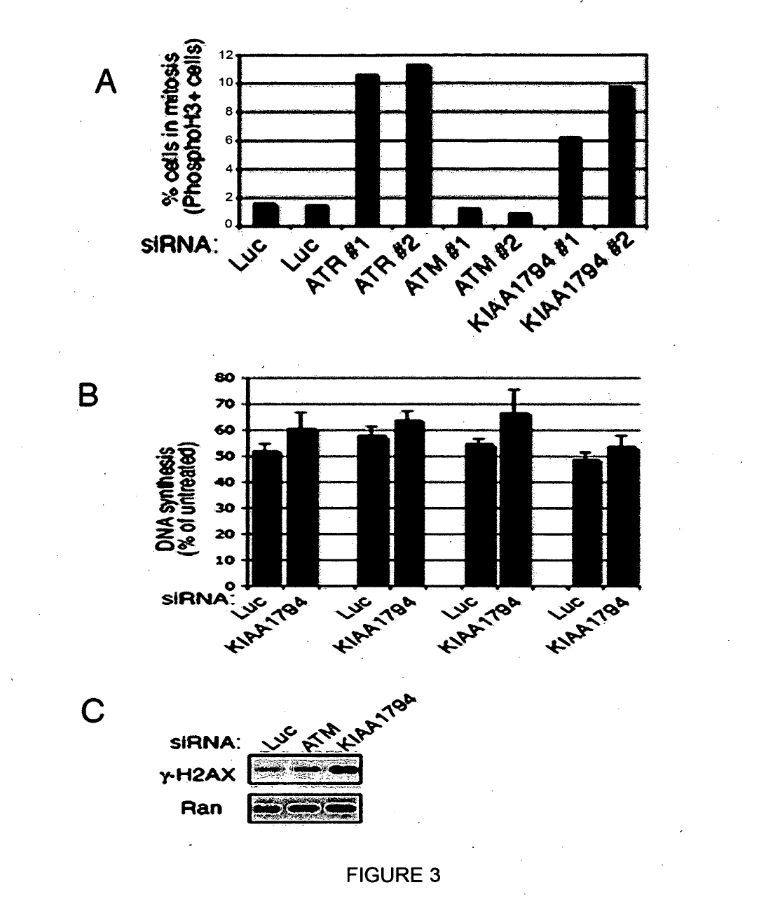

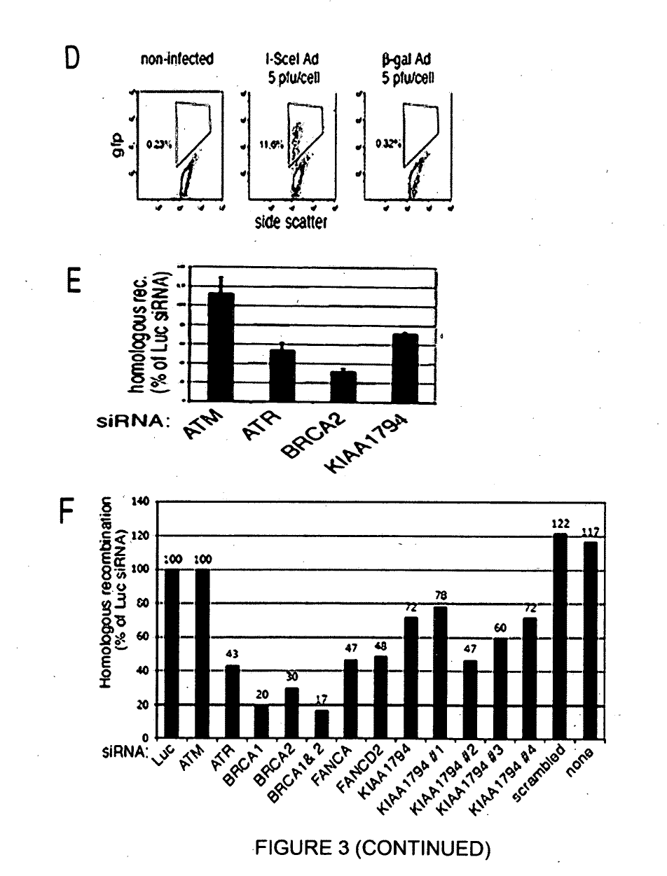

ATM/ATR pathways control multiple cellular responses. It was examined if FANCI participated in cell cycle control, DNA synthesis control, or homologous recombination following DNA damage. siRNA against FANCI abrogated the G2/M checkpoint in U2OS cells (refer to FIG. 3A) and also had a small but reproducible effect in the intra-S phase checkpoint (refer to FIG. 3B). (FIG. 3A shows that cells depleted for FANCI have checkpoint defects. For the experiments presented in FIG. 3A, U2OS cells were treated as shown in the schematic. Two separate fields of cells were examined. The mean and standard deviation from two fields are shown, and an average of 1000 cells per siRNA were scored. FIG. 3B demonstrates the effects of FANCI depletion on radio-resistant DNA synthesis. For the experiments presented in FIG. 3A, U2OS cells transfected with the indicated combination of three different siRNAs were irradiated with 5Gy or 10 Gy of γ-IR depending on an experiment, allowed to recover for 30 minutes and assayed in triplicate for DNA synthesis. The means and standard deviations of four separate experiments are shown. For comparison, IR treatment of the ATM siRNA-transfected cells caused DNA synthesis to be 70-80% of the level found in the untreated cells.) Interestingly, in unirradiated cells, FANCI depletion caused an increased basal level of damage as judged by γ-H2AX (refer to FIG. 3C, demonstrating that reduction of FANCI caused spontaneous DNA damage. In FIG. 3C, U2OS cells transfected with the indicated combinations of three different siRNAs were collected three days later and the level of γ-H2AX was assayed without inflicting any exogenous damage. Western analysis with Ran antibody acted as a loading control.), indicative of a role in maintenance of genomic stability.

The FA pathway has been previously implicated in homologous recombination (HR; Nakanishi et al. (2005) Proc Natl Acad Sci USA 102: 1110-1115; Niedzwiedz et al. (2004) Mol Cell 15: 607-620; Yamamoto et al. (2005) Mol Cell Biol 25: 34-43). FANCI was examined for a role in HR repair. DR-U2OS cells used in this assay (Xia et al. (2006) Mol Cell 22: 719-729) have an integrated HR reporter. Induction of a double-strand break resulted in a robust repair, as demonstrated by the appearance of 12% GFP positive cells (refer to FIG. 3D, showing the results of flow cytometric analysis of DR U2OS cells uninfected or infected with the AdNgus24i adenovirus carrying I-SceI (1-SceI-Ad) or AdCA36 carrying β-galactosidase (β-gal-Ad). For the experiments of FIG. 3D, infections were carried out at an M.O.I. of 5 and analysis for gfp positive cells was performed at 36 hours after infection.). All four siRNAs to FANCI reduced recombination from 78% to 47% of controls, similar to siRNAs to ATR, FANCA and FANCD2 (Nakanishi et al. (2005) Proc Natl Acad Sci USA 102: 1110-1115) but less than siRNAs to BRCA1 and BRCA2, which are thought to be more directly involved in the recombination process (refer to FIGS. 3E and 3F, which show that FANCI was required for homologous recombination. For the experimental results shown in FIG. 3E, DR U2OS cells were transfected with the indicated combination of three different siRNAs and three days later were infected with 10 pfu/cell of adenovirus carrying I-SceI. Flow cytometric analysis of gfp positive cells was carried out 36 hours after infection. Mean and standard deviation of 8 experiments (ATM), 7 experiments (ATR), 4 experiments (Brca2) and 3 experiments (FANCI) are presented in FIG. 3E. For the experimental results shown in FIG. 3F, DR U2OS cells were transfected with the indicated individual siRNAs, infected with 5 pfu/cell of adenovirus carrying I-SceI (AdNgus24i) and analyzed 24 hours later.). These results demonstrated FANCI to be an important component of the HR repair pathway.

### Example 6

### FANCI Localized to Damage-Induced Foci in Multiple Cell Types

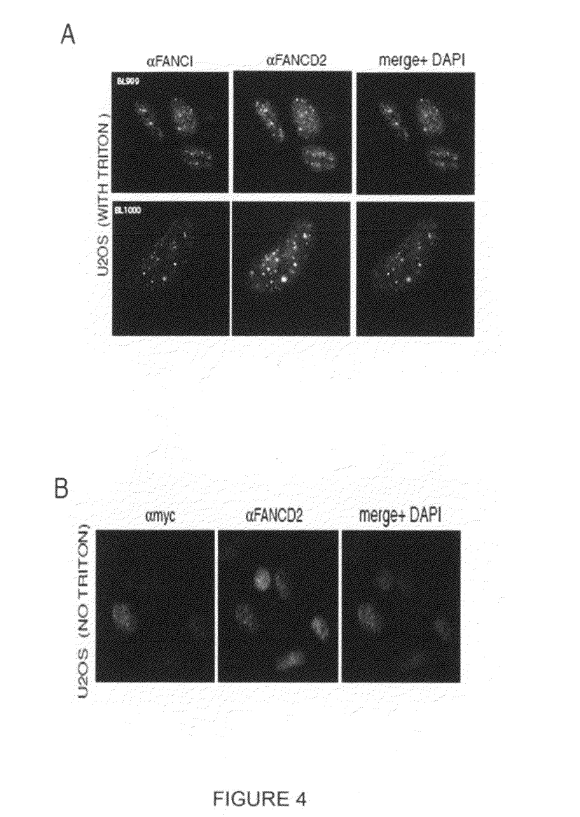

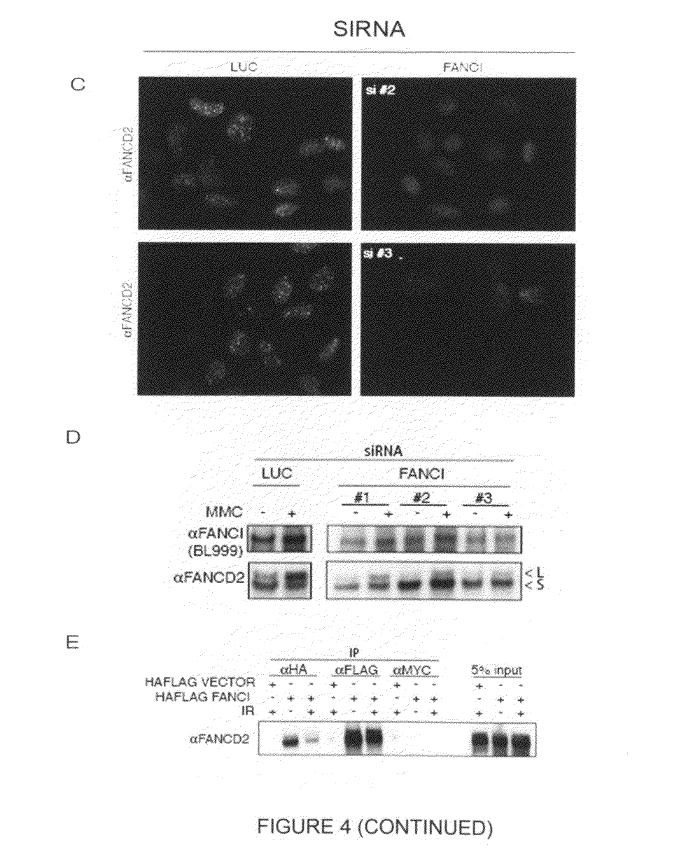

To assess FANCI localization, immunofluorescence experiments were performed on transformed (U2OS, HeLa, and 293T) and primary (BJ) cell lines. Analysis using two antibodies, BL999 and BL1000, revealed foci in a subset of untreated cells and in nearly all cells after DNA damage. In some experiments, a nuclear rim staining was also detected. These FANCI foci corresponded to damage-induced foci as they colocalized with FANCD2 staining (refer to FIG. 4A, showing the localization of endogenous FANCI using BL999 and BL1000 antibodies; Garcia-Higuera et al. (2001) Mol Cell 7: 249-262; for the results shown in FIG. 4A, U2OS cells treated with 1 μM mitomycin C for 24 hours were triton-extracted before co-staining with anti-FANCI (BL999 or BL1000) and anti-FANCD2 antibodies). Confirmation of the antibody specificity was achieved using transfected Myc-FANCI and anti-Myc antibodies (refer to FIG. 11A, showing the localization of exogenous myc-tagged FANCI. For the results shown in FIG. 11A, U2OS cells were transduced with a Myc-tagged FANCI-carrying retrovirus and treated with 1 μM mitomycin C. 24 hours later cells were co-stained with 9E10 antibody (Myc) and a rabbit antibody against human FANCD2 without triton pre-extraction). siRNA-treated cells showed decreased damage-induced foci staining with BL999 and BL1000 antibodies after Triton pre-extraction (data not shown).

### Example 7

### FANCI and FANCD2 were Identified to Form a Complex Required for FANCD2 Localization to Damage-Induced Foci

Depletion of FANCI in U2OS using three separate siRNAs resulted in diminished ubiquitination of FANCD2 upon damage (refer to FIG. 4C, showing Western analysis of FANCD2 in U2OS cells transfected with individual siRNAs against FANCI) and the loss of this modification corresponded to a prominent reduction in FANCD2 signal at damage-induced foci as well as appearance of cells with no visible FANCD2 foci (refer to FIG. 4B, showing the localization of FANCD2 in cells transfected with individual siRNAs against FANCI). (For the experimental results shown in FIG. 4B, U2OS cells were transfected with the indicated individual siRNAs against FANCI and treated with 1 μM mitomycin C. Twenty-four hours later, following triton extraction, the cells were co-stained with an antibody against FANCD2 and H2AX. For the results shown in FIG. 4C, “L” indicates the long (monoubiquitinated) form while “S” indicates the short form of the proteins. The asterisk (*) in FIG. 4C indicates a cross-reacting band.) Moreover, the steady state level of FANCD2 was decreased upon depletion of FANCI (refer to FIG. 4C). There was also a reciprocal relationship between FANCD2 and FANCI since the knockdown of FANCD2 also led to decreased foci formation of FANCI (refer to FIG. 11B, top panel, showing localization of FANCI in cells transfected with 2 different siRNAs against FANCD2. For the results shown in FIG. 11B, U2OS cells were transfected with the indicated individual siRNAs against FANCD2 and treated with 1 μM mitomycin C. Twenty-four hours later following 0.5% triton extraction the cells were stained with an antibody against FANCI, FANCD2, or H2AX.). Loss of FANCD2 upon depletion of FANCI was likely attributable to the two proteins being found in a complex. Immunoprecipitation of HA-FLAG-tagged FANCI expressed in 293T cells with antibodies against either HA or FLAG, but not MYC, resulted in co-immunoprecipitation of endogenous FANCD2 (refer to FIG. 11C, showing results of treating 293T cells stably transduced with a HA-FLAG FANCI retrovirus with 10 Gy of γ-IR. For the results shown in FIG. 11C, 1 mg total protein was immunoprecipitated with HA, FLAG or Myc antibodies. The immunoprecipitates were analyzed by western blotting with a rabbit anti-FANCD2 antibody). The interaction of FANCI and FANCD2 was independent of DNA damage and was robust, with 15-20% of total FANCD2 immunoprecipitated. Immunoprecipitation of endogenous FANCI was also able to co-immunoprecipitate FANCD2 (refer to FIG. 11D, showing the interaction of FANCD2 and endogenous FANCI) and immunoprecipitation with FANCD2 antibodies recovered FANCI (refer to FIG. 11E, showing the interaction of FANCD2 and FANCI within a FANCD2 IP). (For the results shown in FIG. 11D, 0.5 mg total protein from PD20 fibroblasts expressing WT or K561R allele of FANCD2 were immunoprecipitated with anti-FANCI antibody (BL1000) under non-damaged conditions. The immunoprecipitates were analyzed by western blotting with a rabbit anti-FANCD2 or rabbit anti-FANCD2 antibody. For the results shown in FIG. 11E, 0.5 mg total protein from PD20 fibroblasts expressing WT or K561R allele of FANCD2 and also expressing HAFLAG-tagged WT or K523R allele was immunoprecipitated with anti-FANCD2 antibodies under non-damaged conditions. The immunoprecipitates were analyzed by western blotting with a rabbit anti-FANCD2 or mouse anti-HA antibody) To test if monoubiquitination of FANCD2 was required for this interaction, PD20 cells complemented with WT FANCD2 or the K561R mutant of FANCD2 (that cannot be monoubiquitinated; Garcia-Higuera et al., 2001) were used in immunoprecipitation experiments. Immunoprecipitation of HA-FLAG-tagged FANCI expressed in these cells recovered both WT FANCD2 and the K561R mutant FANCD2 (refer to FIG. 4D, lanes 8 and 9, showing the interaction of FANCD2 and FANCI), demonstrating that ubiquitination of FANCD2 was not required for the interaction of FANCD2 with FANCI. (For the results shown in FIG. 4D, total protein (0.5 mg) from PD20 fibroblasts expressing indicated constructs was immunoprecipitated with FLAG or control Myc antibodies under non-damaged conditions. The immunoprecipitates were analyzed by western blotting with a rabbit anti-FANCD2 or mouse anti-HA antibody.)

### Example 8

### Ubiquitinated FANCI Appeared After Damage During an Unperturbed S Phase

It was examined if FANCI was ubiquitinated at a conserved lysine residue in FANCI corresponding to the FANCD2 ubiquitination site. A slower migrating band present on western blots performed with two non-overlapping anti-peptide antibodies in U2OS cells (refer to FIG. 5A, showing western blot analysis of FANCI in U20S cells) as well as in other cell lines including primary BJ fibroblasts (refer to FIG. 5 and data not shown) indicated that a modified form of FANCI was present in these cells. The slower migrating band (long form, L), although present in the untreated cells, increased in intensity following DNA damage inflicted by MMC (refer to FIG. 5A) or HU (refer to FIGS. 5G and H, respectively showing analysis of ubiquitination in PD20 (FA-D2) fibroblasts and ubiquitination of FANCD2 and FANCI in HeLa cells transfected with siRNA against USP1 and LacZ control, treated with 2 mM HU and collected 15 hours later). The molecular weight difference between the long form and the short form (S) was consistent with monoubiquitination. To directly test for ubiquitination, FANCI was immunoprecipitated from 293T cells expressing HA-tagged ubiquitin and immunoblotted with HA-antibodies (refer to FIG. 5B, showing in vivo ubiquitination of FANCI). A band of appropriate size was identified that corresponded to the long form of FANCI was found only in cells transfected with HA-tagged ubiquitin but not in control cells. To exclude the possibility that the monoubiquitinated protein seen in FIG. 5B was a FANCI-associated protein, pulldowns from HeLa extracts expressing His-biotin-ubiquitin were performed with Streptavidin under fully denaturing conditions (Tagwerker et al. (2006) Mol Cell Proteomics 5: 737-748). WT FANCI protein but not K523R FANCI mutant protein (see below) precipitated under these conditions (refer to FIG. 5C, showing in vivo ubiquitination of FANCI in HeLa cells). Accordingly, FANCI protein, like FANCD2 protein, was identified to be monoubiquitinated in vivo. (For the results shown in FIG. 5A, U2OS cells were treated with 1 μM MMC and 24 hour later cells were lysed directly in 2× Laemmlie buffer. Long (L) and short (S) forms of FANCI are shown in FIG. 5A, and the asterisk indicates a cross-reacting band. For the results shown in FIG. 5B, whole cell extracts of 293T cells transiently transfected with HA-tagged ubiquitin or control plasmid carrying dsRed marker were immunoprecipitated using antibodies raised against FANCI and analyzed by western blot with a FANCI antibody (left) and antibody recognizing the HA tag (right). For FIG. 5C, HeLa cells expressing ubiquitin tagged with His and a biotynylation signal were treated with 2 mM HU for 16 hours, lysed in 8M urea and precipitated using Streptavidin beads under denaturing conditions. For FIG. 5G, cells expressing vector, K561R mutant or WT FANCD2, were treated with 2 mM HU and collected 15 hours later. Western blotting was performed with the indicated antibodies including FANCD2 antibody to confirm absence (lane 1 and 2) or presence (lanes 3, 4, 5, and 6) of FANCD2 protein, and the asterisk in FIG. 5G indicates a cross-reacting band. For FIG. 5H, L/S indicates the ratio of the monoubiquitinated to non-ubiquitinated FANCI or FANCD2.)

Chromatin fractionation experiments revealed that the ubiquitinated form of FANCI, like FANCD2 (Montes de Oca et al. (2005) Blood 105, 1003-1009), was enriched in chromatin (refer to FIG. 5D, showing chromatin fractionation of FANCI in U2OS cells. For the results shown in FIG. 5D, cells were treated with 1 μM MMC and 24 hours later cells were collected and processed into cellular fractions. Whole cell extract (WCE), cytoplasmic proteins (S1), intact nuclei (P1), soluble nuclear proteins (S2), chromatin-enriched pellet (P2), soluble and insoluble fractions after micrococcal nuclease treatment (S2′ and P2′) are indicated. Orc2 antibody was used to follow the chromatin fraction). To examine whether FANCI was modified during the cell cycle, U2OS cells were synchronized and released from a mitotic block. Cells in mitosis and G1 phase of cell cycle lacked ubiquitinated FANCI or FANCD2 proteins (refer to FIG. 5E, showing cell cycle analysis of FANCI ubiquitination; for the results shown in FIG. 5E, after release from nocodazole, cells were collected at indicated times for the western analysis (top panel) and for cell cycle analysis using flow cytometry (lower panel)). By 9 hours after release, when most cells were in early S phase, FANCI appeared ubiquitinated. Because the experiment was performed in the absence of exogenous damage, it was concluded that endogenous FANCI was modified in an unperturbed S phase.

### Example 9

### Ubiquitination of FANCI was Identified to be FANCA and FANCD2 Dependent

To search for the E3 ubiquitin ligase for FANCI, FANCI modification was examined in FANCA mutants defective for the core E3 ligase complex. FA-A cells GM6914 cells lacking FANCA showed no ubiquitination of the endogenous or HA-tagged FANCI (refer to FIG. 5F, lanes 1, 2, 5, and 6, showing analysis of ubiquitination in GM6914 (FA-A) fibroblasts) but ubiquitination was restored after complementation with WT FANCA (refer to FIG. 5F, lanes 3, 4, 7, 8). (For the results shown in FIG. 5F, cells expressing vector or WT FANCA were stably transduced with empty vector, or HA-tagged WT FANCI. Twenty-four hours after 1 μM MMC treatment, cells were collected and western blotting was performed with the indicated antibodies.)

FANCD2 and FANCI showed reciprocal ubiquitination dependencies. PD20 fibroblasts, which lack FANCD2 (Jakobs et al. (1996) Somat Cell Mol Genet. 22: 151-157), when transfected with the ubiquitination-defective FANCD2 K561R mutant also failed to ubiquitinate FANCI (refer to FIG. 5G, lanes 3 and 4). The same cells complemented with WT FANCD2 restored FANCI modification (refer to FIG. 5G, lanes 5 and 6). PD20 cells expressing WT or K561R FANCD2 also showed increased levels of FANCI (refer to FIG. 5G), indicating that the non-ubiquitinated forms of the protein bound constitutively in a heterodimeric (or multimeric) Fanconi anemia ID complex.

USP1 was previously identified as the deubiquitinating enzyme for FANCD2 (Nijman et al. (2005) Mol Cell 17: 331-339). To test whether USP1 could also affect FANCI monoubiquitination, HeLa cells were transfected with siRNA against USP1. Reduction of USP1 increased the L to S ratio (ubiquitinated form to deubiquitinated form ratio) for both FANCI and FANCD2 under basal conditions and after KU treatment. (refer to FIG. 5H).

### Example 10

### Lysine 523 Was Identified as Critical for FANCI Ubiquitination

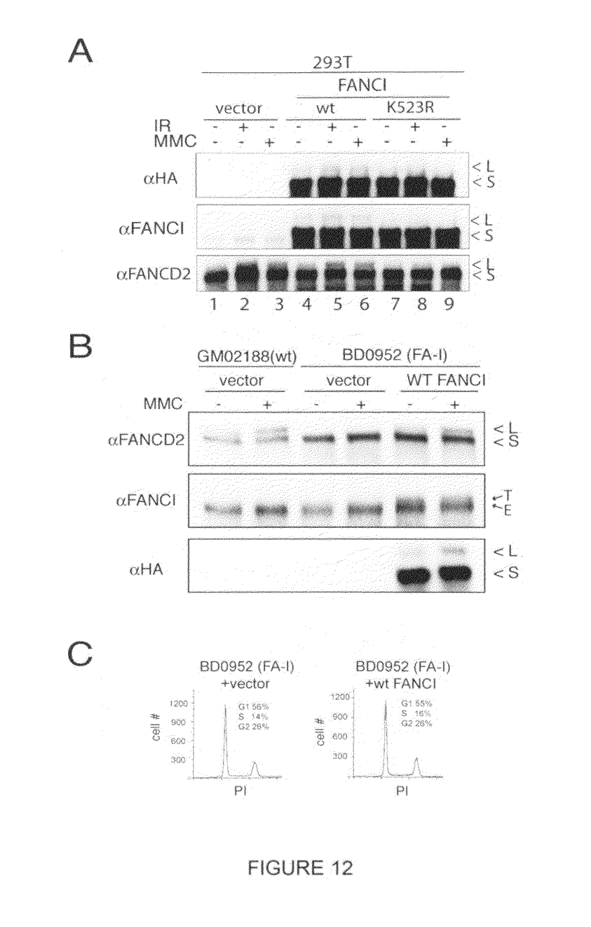

To determine whether the conserved lysine 523 of FANCI was required for ubiquitination, a WT or K523R mutant HA-tagged FANCI was stably expressed in GM6914 (refer to FIG. 5F) and in 293T cells (refer to FIG. 12A, showing lack of ubiquitination of K523R FANCI). Only in cells that expressed the WT FANCI but not the K523R mutant was the L form (ubiquitinated form) detected with the HA antibody (refer to FIG. 5F, lanes 7, 8, 11, 12 and to FIG. 12A). Interestingly, cells overexpressing the FANCI K523R mutant, but not WT, showed diminished monoubiquitination of FANCD2 (refer to FIG. 5F, lanes 11, 12 and to FIG. 12A), indicating that the mutant FANCI displays a dominant negative activity. (For the results shown in FIG. 12A, 293T cells were stably transduced with HA-FLAG-tagged WT or K523R FANCI alleles. 8.5 hours after 15Gy IR or 1 μM MMC treatment cells were harvested and lysed in Laemmli buffer.)

Consistent with the role of ubiquitination of FANCD2, the FANCI K523R mutant failed to form DNA damage foci (refer to FIG. 5I +TRITON panel), despite its overproduction (data not shown). Cells expressing K523R FANCI allele showed pan-nucleoplasmic FANCD2 staining and greatly diminished localization to DNA damage-induced foci best visualized after triton pre-extraction (refer to FIG. 5I, showing localization of FANCI and FANCD2 in WT and K523R FANCI-expressing U20S cells). These data showed that the K523R mutant had a dominant negative effect on FANCD2 foci formation. (For the experimental results shown in FIG. 5I, cells stably transduced with the HA-tagged WT or K523R mutant allele of FANCI were treated with 1 μM MMC and processed 24 hours later for immunofluorescence. It is noted that cells not expressing K523R in the lower panels (K523R-triton) were included as controls for FANCD2 staining. Two FANCD2 positive cells in the lower right panel (+triton) were presumed not to have K523R FANCI expression, although that cannot be tested directly because triton removes nucleoplasmic FANCI. Similar results were observed in U2OS cells expressing the K523R mutant treated with HU.)

### Example 11

### FANCI was Identified as Mutated in Cells from the Fanconi Anemia Complementation Group I

Phenotypic similarities of cells with reduced levels of FANCI to cells from Fanconi anemia patients, including marked MMC but only mild IR sensitivity (refer to FIG. 1B) indicated that mutations in FANCI were likely responsible for human disease. Published reports included only one remaining complementation group for which the responsible gene was unknown, Fanconi anemia complementation group I (Levitus et al. (2004) Blood 103: 2498-2503). A cell line from this group was obtained, named BD0952, which was an EBV-transformed cell line derived from peripheral lymphocytes of a patient with a classic presentation of Fanconi anemia. BD0952 was identified to express a full-length FANCI protein at normal levels relative to control cells (GM03288; refer to FIG. 6A, showing complementation of FANCD2 ubiquitination defects in FA-I cells by expression of WT FANCI). However, this protein is not ubiquitinated in BD0952 cells, even after Mitomycin C treatment (refer to FIG. 6A and data not shown). (For the results shown in FIG. 6A, cells stably transduced with empty vector, HA-tagged WT or K523R FANCI, were untreated or treated with 100 nM MMC and collected 24 hours later by lysis in Laemmli buffer. Western analysis with FANCD2, FANCI, and HA antibodies was performed. GM02188 (WT control) cells acted as a control for the presence of long (L, ubiquitinated) forms of FANCD2 and FANCI, which were absent in the uncomplemented BD0952 cells. The transduced form of the protein is indicated as T (tagged) because it runs slightly slower than the endogenous (E) form. Also see FIG. 12B, which shows a similar experiment to show that the WT HA-tagged FANCI became ubiquitinated. The exposure for the western blot performed with the FANCI antibody was not high enough to see the long form of FANCI in this blot. However, the transduced form of the protein was identifiable (T for tagged) because it ran slightly slower than the endogenous (E) form. The long form of FANCI was visible when probed with an antibody recognizing HA tag.)

It had previously been shown that FANCD2 was not ubiquitinated in FA-I cells (FIG. 6A and (Levitus et al. (2004) Blood 103: 2498-2503)). Thus, restoration of FANCD2 ubiquitination was employed as a surrogate marker for the functional complementation of the Fanconi anemia pathway. Expression of the FANCI cDNA in BD0952 cells was found to restore FANCD2 ubiquitination (refer to FIG. 6A). This exogenous FANCI was also monoubiquitinated (refer to FIG. 6A and 12B). Appearance of the monoubiquitination was not due to changes in the cell cycle of the cells expressing FANCI (refer to FIG. 12C, showing cell cycle analysis of BD0952 complemented with an empty vector or with WT FANCI, where cells stably transduced with HA-tagged WT FANCI or with empty vector were stained with PI and the cell cycle stage was assessed by flow cytometry). Also, the levels of expression of the exogenous proteins were comparable the endogenous protein (compare T [tagged] vs. E [endogenous] in FIGS. 6A and 12B). Expression of WT FANCI in BD0952 also complemented their MMC resistance to WT levels (refer to FIG. 6B, showing complementation of MMC sensitivity of BD0952 cells by expression of WT FANCI but not empty vector). (For the experimental results shown in FIG. 6B, logarithmically growing cells of indicated genotypes were treated in triplicate with different levels of MMC ranging from 0 to 100 nM. The cells were allowed to grow for 6 days at which time they were harvested and total cell number was counted using a coulter counter. Total cell numbers at each dose were divided by the number of cells in the untreated sample to arrive at percent survival.)

To look for FANCI mutations in BD0952 cells, the cDNA from BD0952 mRNA was amplified and sequenced, resulting in the identification of two base substitutions as candidates for the Fanconi anemia-causing mutation in BD0952 cells. These mutations included a C to T transition which resulted in a Pro to Leu change at amino acid 55, and a G to A transversion which resulted in an Arg to Glu substitution in an absolutely conserved Arg1285 at the C-terminus of the protein. These mutations were confirmed by amplifying exon 3 and exon 36 from genomic DNA. Sequencing confirmed the presence of both mutations in genomic DNA in homozygous form (refer to FIG. 6C, showing sequence analysis of the FANCI genomic locus in BD0952 (FA-I) cells.). Homozygosity was expected at the disease locus of BD0952 cells since the patient from whom the cells were derived is a member of a consanguineous family where both parents were expected to have contributed the disease allele of FANCI to the patient. (For the sequence analysis of FIG. 6C, sequence of the genomic contig (ref|NT_{—}010274.16|Hs15_{—}10431:4714523-4889523 Homo sapiens chromosome 15 genomic contig, reference assembly), and sequence and sequence traces of genomic DNA from BD0952 cells were depicted together with the resulting amino acid sequence deduced from the DNA sequence data).

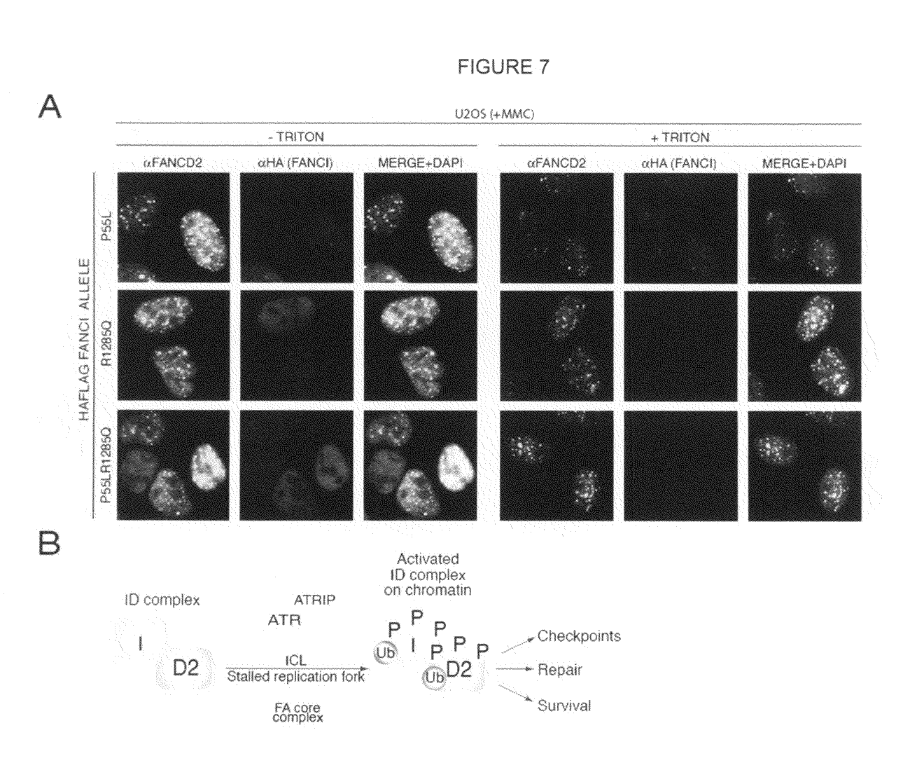

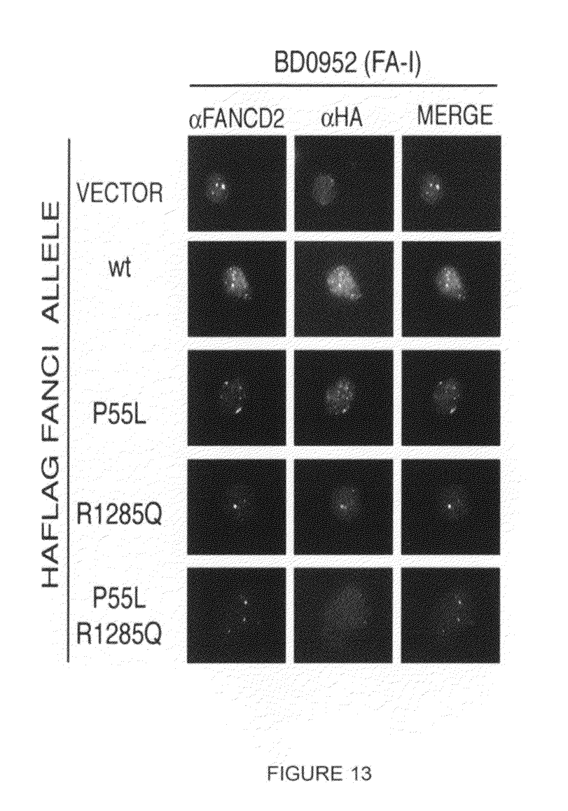

To identify which mutation caused Fanconi anemia, expression constructs were made that contained P55L, R1285Q, and P55L&R1285Q substitutions. Only WT FANCI and the P55L FANCI allele were observed to complement the FANCD2 monoubiquitination defect (refer to FIG. 6D, showing complementation of FANCD2 ubiquitination by expression of WT FANCI or P55L FANCI, but not R1285Q or P55L, R1285Q FANCI mutants; in these experiments, cells stably transduced with the indicated alleles of FANCI were left untreated or were treated with 100 nM of MMC and processed 24 hours later as indicated in panel A) and MMC sensitivity (refer to FIG. 6E, showing complementation of MMC sensitivity of BD0952 cells by expression of WT FANCI or P55L FANCI, but not R1285Q or P55L, R1285Q FANCI mutants; experiments were performed as described for results shown in FIG. 6B) of BD0952 cells. These two proteins were also themselves monoubiquitinated in BD0952 cells. When introduced into U2OS cells or into BD0952 cells, the P55L allele was found in foci together with FANCD2 (refer to FIGS. 7A and 13). Cells expressing R1285Q or P55L&R1285Q alleles showed no monoubiquitination of FANCD2 (refer to FIG. 6D) and failed to restore MMC-resistance (refer to FIG. 6E). Unlike the WT FANCI allele, which could complement the breakage phenotype observed in BD0952 cells, the R1285Q allele-expressing cells showed a high number of aberrations following treatment with MMC (refer to FIG. 6F). When introduced into U2OS or BD0952 cells, the R1285Q or P55L&R1285Q alleles failed to localize to damage-induced foci (refer to FIG. 7A, +TRITON panel, showing the localization of WT, P55L, R1285Q, and P55L, R1285Q mutant proteins in U2OS cells, and to FIG. 13, showing the localization of same in BD0952 (FA-I) cells) despite robust expression levels of the mutant proteins as judged by the immunofluorescence staining in the absence of triton extraction (refer to FIG. 7A-TRITON panel). Together, these studies demonstrated that the R1285Q change is the disease causing mutation in BD0952 cells. (For the results shown in FIG. 7A, U20S cells transduced with the indicated alleles of FANCI were treated with 100 nM MMC and 24 hours later were processed for immunofluorescence. Note that FIG. 7B presents a model of Fanconi anemia ID complex regulation and function. The phosphorylation-ubiquitination cascade culminates in chromatin loading of the Fanconi anemia ID complex, which directs downstream repair events. For the results shown in FIG. 13, BD0952 transduced with the WT and mutant alleles of FANCI were treated with 100 nM MMC and 24 hours were processed for immunofluorescence. It is noted that BD0952 that were not complemented still contained some FANCD2 foci. However, these cells were much fewer in number and they were large and amorphous, unlike the foci that formed after complementation with the WT or P55L FANCI allele.)

Unexpectedly, the K523R FANCI allele was identified to partially complement the FANCD2 monoubiquitination defect (refer to FIG. 6A, lanes 7 and 8) and MMC sensitivity defect in BD0952 cells (refer to FIG. 6F, showing cytogenetic abnormalities in BD0952 cells cells expressing WT, K523R or R1285Q FANCI alleles). (For the results shown in FIG. 6F, indicated cells were treated with 0, 20 or 40 ng MMC per ml of media and analyzed for presence of chromosomal aberrations 48 hours later. The K523R mutant was not assessed at 20 ng of MMC per ml. Analysis was done only once at 40 ng of MMC per ml. 30-50 metaphases were evaluated for each cell line.) This is in contrast to the findings that the FANCI K523R mutant failed to be ubiquitinated or form damage-induced foci and that the K523R allele when overexpressed acted as a dominant negative against FANCD2 ubiquitination and foci formation. These results indicated that either this allele was only partially defective or, more likely, that it was displaying interalleleic complementation with the FANCI R1285Q mutant present in BD0952 cells.

### Example 12

### Identification and Characterization of Potential Inhibitors of FANCI Ubiquitination and Foci Formation

Using the microscopy methods described above and, e.g., a labeled FANCI polypeptide and/or an anti-FANCI antibody (e.g., BL999 or BL1000 (Bethyl)), test compounds (e.g., the 489 known bioactive compounds within the collection of the Institute of Chemistry and Cell Biology (ICCB), Harvard Medical) are screened for inhibition of IR-mediated FANCI foci formation. Positives are identified using a primary screen, which employs high throughput fluorescence microscopy to identify agents which block the formation of FANCI-containing foci upon exposure to ionizing radiation. Candidate compounds are identified and characterized as described, e.g., in U.S. application Ser. No. 11/441,289, the contents of which are incorporated herein by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of diagnosing or determining if a subject has cancer or is at increased risk of cancer, the method comprising testing a sample from the subject for the presence of FANCI-containing foci using an antibody or antigen binding fragment thereof specific for FANCI, wherein said presence of FANCI-containing foci is indicative of cancer or an increased risk of cancer in said subject.

2. The method of claim 1, wherein the antibody or antigen binding fragment thereof is selected from the group consisting of a monoclonal antibody and a polyclonal antibody.

3. The method of claim 1, wherein the antibody or antigen binding fragment thereof is an anti-KIAA1794 antibody selected from the group consisting of BL999 and BL1000.

4. The method of claim 1, wherein the antibody or antigen binding fragment thereof is detectably labeled.

5. The method of claim 4, wherein the detectable label is selected from the group consisting of a radioactive, enzymatic, biotinylated and fluorescent label.

6. The method of claim 1, wherein the sample is derived from a tissue selected from the group consisting of heart, brain, placenta, liver, skeletal muscle, kidney, pancreas, spleen, thymus, prostate, testis, uterus, small intestine, colon, peripheral blood and lymphocytes.

7. The method of claim 1, wherein the sample is selected from the group consisting of a blood sample from the subject, a biopsy sample of tissue from the subject and a cell line.

8. The method of claim 1, wherein the cancer is selected from the group consisting of melanoma, leukemia, astocytoma, glioblastoma, lymphoma, glioma, Hodgkins lymphoma, chronic lymphocyte leukemia and cancer of the pancreas, breast, thyroid, ovary, uterus, testis, pituitary, kidney, stomach, esophagus and rectum.

9. A method of diagnosing or determining if a subject has cancer or is at increased risk of cancer, the method comprising testing a FANCI gene of the subject for the presence of a cancer-associated coding change, wherein said presence of one or more cancer-associated coding changes is indicative of cancer or an increased risk of cancer in the subject.

10. The method of claim 9, wherein the cancer-associated coding change encodes a change in the FANCI polypeptide at a position selected from the group consisting of K523, K1269, R1285, S730, T952, S1121, and P55.

11. The method of claim 10, wherein the change in the FANCI polypeptide is R1285Q.

12. The method claim 1, wherein the subject is human.

13. A method of determining if a subject has cancer, or is at increased risk of developing cancer, said method comprising the steps of:
(a) providing a DNA sample from said subject;
(b) amplifying the FANCI gene from said subject with any of the FANCI gene-specific polynucleotide primers shown in Example 1;
(c) sequencing the amplified FANCI gene; and
(d) comparing the FANCI gene sequence from said subject to a reference FANCI gene sequence, where a discrepancy between the two gene sequences indicates the presence of a cancer-associated defect,
wherein the presence of one or more cancer-associated defects indicates said subject has cancer or is at an increased risk of developing cancer.

14. The method of claim 13, wherein the patient has no known cancer causing defect in the BRCA 1 or BRCA-2 genes.

15. A method of diagnosing or determining if a subject has Fanconi anemia or is at increased risk of developing Fanconi anemia, the method comprising testing a FANCI gene of the subject for the presence of a Fanconi anemia-associated coding change, wherein said presence of one or more Fanconi anemia-associated coding changes is indicative of Fanconi anemia or an increased risk of Fanconi anemia in the subject.

16. A method of determining if a subject has cancer, or is at increased risk of developing cancer comprising the steps of:
(a) providing a DNA sample from said subject;
(b) amplifying the FANCI gene from said subject with FANCI gene-specific polynucleotide primers;
(c) sequencing the amplified FANCI gene; and
(d) comparing the FANCI gene sequence from said subject to a reference FANCI gene sequence, wherein a discrepancy between the two gene sequences indicates the presence of a cancer-associated coding change,
wherein the presence of one or more cancer-associated coding changes indicates said subject has cancer or is at an increased risk of developing cancer.

17. The method of claim 16, wherein the FANCI gene-specific polynucleotide primers are selected from the group consisting of SEQ ID NOs: 1-8.

18. A method of predicting whether a subject with a neoplastic disorder will respond to a genotoxic anti-neoplastic agent comprising determining the size or number of FANCI-containing foci in a sample from the subject using an antibody or antigen binding fragment thereof specific for FANCI, wherein if the number or size of said foci is reduced relative to the number or size of said foci in a sample from a control subject, then the subject is predicted to respond to a genotoxic anti-neoplastic agent.

19. The method of claim 18, wherein the subject was exposed to the genotoxic anti-neoplastic agent prior to the sample being obtained from the subject.

20. The method of claim 19, wherein said exposure is less than or equal to a therapeutically effective dose.

21. The method of claim 19, wherein said exposure is at about 50% or less of the therapeutically effective dose.

22. The method of claim 18, wherein the sample was exposed to the genotoxic anti-neoplastic agent prior to determining the number or size of said foci.

23. The method of claim 18, wherein the genotoxic anti-neoplastic agent is selected from the group consisting of 1,3-bis(2-chloroethyl)-1-nitrosourea, busulfan, carboplatin, carmustine, chlorambucil, cisplatin, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, oxaliplatin, temozolomide, topotecan, and ionizing radiation.

24. The method of claim 18, wherein the number or size of said foci in a sample from the subject is less than about 70% of the number or size of said foci in a sample from a control subject.

25. A method of predicting whether a subject with a neoplastic disorder will respond to a genotoxic anti-neoplastic agent comprising determining the degree of ubiquitination of FANCI polypeptide in a sample from the subject, wherein if the degree of ubiquitination of said FANCI polypeptide in the sample is reduced when compared with a sample from a control subject, then the subject is predicted to respond to a genotoxic anti-neoplastic agent.

26. The method of claim 25, wherein the degree of monoubiquitination of FANCI polypeptide is determined by immunoblot analysis using an antibody or antigen binding fragment thereof specific for FANCI.

27. A method of identifying a tumor that is sensitive to a genotoxic anti-neoplastic agent comprising determining the size or number of FANCI-containing foci in a sample from a test subject, wherein if the number or size of said foci is reduced relative to the number or size of said foci in a sample from a control subject, then the sample from the test subject is identified as a tumor sensitive to a genotoxic anti-neoplastic agent.

28. A method of identifying an inhibitor of a Fanconi anemia DNA repair pathway, comprising:
(a) contacting a cell with a test compound;
(b) contacting the cell with a genotoxic anti-neoplastic compound before, after, or simultaneous with step (a);
(c) quantifying FANCI-containing foci in the cell using an antibody or antigen binding fragment thereof specific for FANCI; wherein if the quantity of said foci is less than in a control cell, wherein the control cell was contacted with said genotoxic anti-neoplastic agent but not with said test compound, then the test compound is identified as an inhibitor of a Fanconi anemia DNA repair pathway.

29. The method of claim 28, further comprising:
(d) for a test compound identified as an inhibitor in step (c), determining the degree of monoubiquitination of FANCI polypeptide in said cell; wherein if the degree of monoubiquitination of FANCI polypeptide is less than in said control cell, then the test compound is further identified as an inhibitor of a Fanconi anemia DNA repair pathway.

30. The method of claim 29, further comprising:
(e) for a test compound further identified as an inhibitor in step (d), contacting a test cell that has a functional Fanconi anemia pathway with said test compound and said genotoxic anti-neoplastic agent;
(f) measuring the sensitivity of the test cell to the genotoxic anti-neoplastic agent; and
(g) comparing the sensitivity of the test cell to the agent to that of a second control cell; wherein the second control cell is isogenic to the test cell but has a defective Fanconi anemia pathway; and wherein if the sensitivity of the test cell is comparable to the sensitivity of the second control cell, the test compound is further identified as an inhibitor of a Fanconi anemia DNA repair pathway.

31. The method of claim 28, wherein a property selected from the group consisting of the number of FANCI-containing foci and the size of FANCI-containing foci is determined in step (c).

32. The method of claim 28, wherein step (c) is performed in high throughput format.

33. The method of claim 29, wherein the degree of monoubiquitination of FANCI polypeptide in step (d) is determined by immunoblot analysis.

34. The method of claim 30, wherein the sensitivity of the test cell and the second control cell to the anti-neoplastic agent is determined by measuring cell survival at one or more concentrations of the anti-neoplastic agent.

35. The method of claim 32, wherein the test cell and the second control cell are human cells.

36. A method of identifying an inhibitor of a non-Fanconi anemia DNA repair pathway, comprising:
(a) contacting a test cell that has a functional Fanconi anemia pathway with a test compound and a genotoxic anti-neoplastic agent;
(b) measuring the sensitivity of the test cell to the genotoxic anti-neoplastic agent; and
(c) comparing the sensitivity of the test cell to the agent to that of a control cell;
wherein the control cell is isogenic to the test cell but has a mutant FANCI gene; and if the sensitivity of the test cell is greater than the sensitivity of the control cell, the test compound is identified as an inhibitor of a non-Fanconi anemia DNA repair pathway.

37. The method of claim 36, wherein the sensitivity of the test cell and the control cell to the anti-neoplastic agent is determined by measuring cell survival at one or more concentrations of the anti-neoplastic agent.

38. The method of claim 36, wherein the test compound does not inhibit the Fanconi anemia pathway.

39. A method of screening for a cancer therapeutic, the method comprising the steps of:
(a) providing one or more cells containing a FANCI gene having one or more cancer associated defects;
(b) growing said cells in the presence of a potential cancer therapeutic; and
(c) determining the rate of growth of said cells in the presence of said potential cancer therapeutic relative to the rate of growth of equivalent cells grown in the absence of said potential cancer therapeutic,
wherein a reduced rate of growth of said cells in the presence of said potential cancer therapeutic, relative to the rate of growth of equivalent cells grown in the absence of said potential cancer therapeutic, indicates that the potential cancer therapeutic is a cancer therapeutic.

40. The method of claim 39, wherein the cells are BD0952 cells.

41. A method of screening for a chemosensitizing agent, said method comprising the steps of:
(a) providing a potential inhibitor of FANCI;
(b) providing a tumor cell line that is resistant to one or more anti-neoplastic agents;
(c) contacting said tumor cell line and said potential inhibitor of FANCI with said one or more anti-neoplastic agents; and
(d) measuring the growth rate of said tumor cell line in the presence of said inhibitor of FANCI and said anti-neoplastic agent,
wherein a reduced growth rate of the tumor cell line, relative to cells of the tumor cell line in the presence of the anti-neoplastic agent and the absence of said inhibitor of FANCI, is indicative that the potential inhibitor is a chemosensitizing agent.

42. A method of sensitizing a subject to treatment with a genotoxic anti-neoplastic agent, the method comprising administering an inhibitor of FANCI to a subject who is receiving a genotoxic anti-neoplastic agent but is resistant to said agent.

43. The method of claim 42, wherein the inhibitor of FANCI is selected from the group consisting of an antibody or antigen binding fragment thereof specific for FANCI and an anti-FANCI RNA interference agent.

44. The method of claim 43, wherein the anti-FANCI RNA interference agent targets a sequence in FANCI selected from the group consisting of SEQ ID NO: 22, SEQ ID NO: 23, and SEQ ID NO: 24.

45. A method of sensitizing a subject to treatment with a genotoxic anti-neoplastic agent, the method comprising:
(a) administering an inhibitor of FANCI to a subject who is receiving treatment with a genotoxic anti-neoplastic agent but is resistant to said agent; and
(b) administering an inhibitor of a non-Fanconi anemia DNA repair pathway to the subject.

46. The method of claim 45, wherein the inhibitor of a non-Fanconi anemia DNA damage repair pathway is selected from the group consisting of a PARP inhibitors, a DNA-PK inhibitor, an mTOR inhibitor, an ERCC1 inhibitor, an ERCC3 inhibitor, an ERCC6 inhibitor, an ATM inhibitor, an XRCC4 inhibitor, a Ku80 inhibitor, a Ku70 inhibitor, an XPA inhibitor, a CHK1 inhibitor, and a CHK2 inhibitor.

47. The method of claim 45, wherein the genotoxic anti-neoplastic agent is administered simultaneously with the inhibitor of FANCI and the inhibitor of a non-Fanconi anemia DNA repair pathway.

48. A method of predicting the efficacy of a therapeutic agent in a cancer patient, comprising the steps of: (a) providing a tissue sample from said cancer patient who is being treated with said therapeutic agent; (b) inducing DNA damage in the cells of said tissue sample; (c) detecting the presence of ubiquitinated FANCI protein in said cells; wherein the presence of ubiquitinated FANCI is indicative of a reduced efficacy of said therapeutic agent in said cancer patient.

49. A kit for determining whether a subject has cancer or is at increased risk of cancer, comprising an antibody or antigen binding fragment thereof specific for FANCI, packaging materials therefor, and instructions for performing the method of claim 1.

50. A kit for determining whether a subject with a neoplastic disorder will respond to a genotoxic anti-neoplastic agent, comprising an antibody or antigen binding fragment thereof specific for FANCI, packaging materials therefor, and instructions for performing the method of claim 18.

51. A kit for identifying an inhibitor of the Fanconi anemia pathway, comprising a test cell and a control cell according to claim 28, and packaging materials therefor.

52. A kit for identifying an inhibitor of a non-Fanconi anemia pathway, comprising a test cell and a control cell according to claim 36, and packaging materials therefor.

53. An isolated nucleotide sequence comprising the mutant FANCI nucleotide sequence of BD0952 cells.

54. An isolated polypeptide sequence comprising a polypeptide sequence selected from the group consisting of the mutant FANCI polypeptide sequence of BD0952 cells shown in FIG. 9 and a GST polypeptide fused to the N-terminal 200 amino acid residues of the FANCI polypeptide.

55. An anti-FANCI siRNA to a FANCI target selected from the group consisting of SEQ ID NO: 22, SEQ ID NO: 23, and SEQ ID NO: 24.


SEQUENCE LISTING (87 sequences)

SEQ ID NO: 1 - DNA, 34 bp, Artificial Sequence
  Description of Artificial Sequence Synthetic primer
ccgctcgaggaccagaagattttatctctagcag

SEQ ID NO: 2 - DNA, 34 bp, Artificial Sequence
  Description of Artificial Sequence Synthetic primer
ccggttaacttaactcaggcatttcatttatttt

SEQ ID NO: 3 - DNA, 24 bp, Artificial Sequence
  Description of Artificial Sequence Synthetic primer
ttcaggattattttggttaggtta

SEQ ID NO: 4 - DNA, 19 bp, Artificial Sequence
  Description of Artificial Sequence Synthetic primer
ggtcacaaatgccctcaag

SEQ ID NO: 5 - DNA, 20 bp, Artificial Sequence
  Description of Artificial Sequence Synthetic primer
tcaaagcccttaaccattgc

SEQ ID NO: 6 - DNA, 20 bp, Artificial Sequence
  Description of Artificial Sequence Synthetic primer
tgccatcttacctccagcat

SEQ ID NO: 7 - DNA, 22 bp, Artificial Sequence
  Description of Artificial Sequence Synthetic primer
tcttgatctgatgacctgaacc

SEQ ID NO: 8 - DNA, 21 bp, Artificial Sequence
  Description of Artificial Sequence Synthetic primer
gtcggggcaacttcataggat

SEQ ID NO: 9 - DNA, 42 bp, Artificial Sequence
  Description of Artificial Sequence Synthetic primer
gcttgatacttgtccttcggcgagctatgtttgccaaccagc

SEQ ID NO: 10 - DNA, 42 bp, Artificial Sequence
  Description of Artificial Sequence Synthetic primer
gctggttggcaaacatagctcgccgaaggacaagtatcaagc

SEQ ID NO: 11 - DNA, 33 bp, Artificial Sequence
  Description of Artificial Sequence Synthetic primer
cttcaaaggttccctctgctctgaggaagctgg

SEQ ID NO: 12 - DNA, 33 bp, Artificial Sequence
  Description of Artificial Sequence Synthetic primer
gctcagcacctcacaagacttcaagatcaaagg

SEQ ID NO: 13 - DNA, 21 bp, Artificial Sequence
  Description of Artificial Sequence Synthetic oligonucleotide
aacgtacgcggaatacttcga

SEQ ID NO: 14 - DNA, 21 bp, Artificial Sequence
  Description of Artificial Sequence Synthetic oligonucleotide
ctggctaatcaccaagcttaa

SEQ ID NO: 15 - DNA, 21 bp, Artificial Sequence
  Description of Artificial Sequence Synthetic oligonucleotide
tcggcaatacttgctatctta

SEQ ID NO: 16 - DNA, 25 bp, Artificial Sequence
  Description of Artificial Sequence Synthetic oligonucleotide
gcgcagtgtagctacttcttctatt

SEQ ID NO: 17 - DNA, 25 bp, Artificial Sequence
  Description of Artificial Sequence Synthetic oligonucleotide
gggcctttgttcttcgagacgttat

SEQ ID NO: 18 - DNA, 25 bp, Artificial Sequence
  Description of Artificial Sequence Synthetic oligonucleotide
gcaacatttgcctatatcagcaatt

SEQ ID NO: 19 - DNA, 25 bp, Artificial Sequence
  Description of Artificial Sequence Synthetic oligonucleotide
gggaaatagtagaacctcatctaaa

SEQ ID NO: 20 - DNA, 25 bp, Artificial Sequence
  Description of Artificial Sequence Synthetic oligonucleotide
ggtctggagtaaagaagccaattta

SEQ ID NO: 21 - DNA, 25 bp, Artificial Sequence
  Description of Artificial Sequence Synthetic oligonucleotide
ccacctgagggtaagaacatgttaa

SEQ ID NO: 22 - DNA, 25 bp, Artificial Sequence
  Description of Artificial Sequence Synthetic oligonucleotide
tctcctcagtttgtgcagatgttat

SEQ ID NO: 23 - DNA, 25 bp, Artificial Sequence
  Description of Artificial Sequence Synthetic oligonucleotide
ggcagctgtgtggacaccttgttaa

SEQ ID NO: 24 - DNA, 25 bp, Artificial Sequence
  Description of Artificial Sequence Synthetic oligonucleotide
gctggtgaagctgtctggttctcat

SEQ ID NO: 25 - DNA, 25 bp, Artificial Sequence
  Description of Artificial Sequence Synthetic oligonucleotide
ttagttgactgacaatgagtcgagg

SEQ ID NO: 26 - DNA, 25 bp, Artificial Sequence
  Description of Artificial Sequence Synthetic oligonucleotide
aatagacgacaacttatccatcacc

SEQ ID NO: 27 - DNA, 25 bp, Artificial Sequence
  Description of Artificial Sequence Synthetic oligonucleotide
aaatgtcactctgagaggatagccc

SEQ ID NO: 28 - DNA, 25 bp, Artificial Sequence
  Description of Artificial Sequence Synthetic oligonucleotide
ttctaacacagcttctagttcagcc

SEQ ID NO: 29 - DNA, 25 bp, Artificial Sequence
  Description of Artificial Sequence Synthetic oligonucleotide
tagagtgctacactgtccaacaccc

SEQ ID NO: 30 - DNA, 25 bp, Artificial Sequence
  Description of Artificial Sequence Synthetic oligonucleotide
ggaagatatcctggctggcactctt

SEQ ID NO: 31 - DNA, 25 bp, Artificial Sequence
  Description of Artificial Sequence Synthetic oligonucleotide
ccagcatattcaggaggccttacta

SEQ ID NO: 32 - DNA, 25 bp, Artificial Sequence
  Description of Artificial Sequence Synthetic oligonucleotide
tccctcctcacagactacatctcat

SEQ ID NO: 33 - protein, 146 aa, Homo sapiens
GKTIETSPSLSRMPNQHACKLGANILLETFKIHEMIRQEILEQVLNRVVTRASSPISHFLDLLSNIVMYAPLVLQSCSSKVTEAFDYLSFLPLQTVQRLLKAVQPLLKVSMSMRDCLILVLRKAMFANQLDARKSAVAGFLLLLKN

SEQ ID NO: 34 - protein, 151 aa, Strongylocentrotus purpuratus
GSLLCIAEKLLRSPEPQVSQFGSKVYQTAFAAFDVYSKQEVVGALVTHIGSGLSAEMDSALDILSSLALSHTKDLAPFAVFIKGVLDYLDNLTMTQVKKLFSVLATLAYRGGSSGSTLQDELHIVVRKNLTSNSPKYKRMGVVAALMVIQN

SEQ ID NO: 35 - protein, 10 aa, Homo sapiens
VLRKAMFANQ

SEQ ID NO: 36 - protein, 10 aa, Xenopus tropicalis
VLQKAIFSRQ

SEQ ID NO: 37 - protein, 10 aa, Danio rerio
VLRKAMFSSQ

SEQ ID NO: 38 - protein, 10 aa, Drosophila melanogaster
VLCKAMSSSG

SEQ ID NO: 39 - protein, 10 aa, Arabidopsis thaliana
VIRKAMFRRE

SEQ ID NO: 40 - protein, 10 aa, Dictyostelium discoideum
VLKKSMFARE

SEQ ID NO: 41 - protein, 10 aa, Homo sapiens
VIRKQLSSTV

SEQ ID NO: 42 - protein, 10 aa, Xenopus tropicalis
VIRKQLSSTV

SEQ ID NO: 43 - protein, 10 aa, Danio rerio
VIRKQLSSTV

SEQ ID NO: 44 - protein, 10 aa, Drosophila melanogaster
VVKKQLINSI

SEQ ID NO: 45 - protein, 10 aa, Dictyostelium discoideum
FTRKQITSTK

SEQ ID NO: 46 - protein, 10 aa, Homo sapiens
IHLSKKSKVS

SEQ ID NO: 47 - protein, 10 aa, Xenopus tropicalis
IHLSKKSKVN

SEQ ID NO: 48 - protein, 10 aa, Danio rerio
IHLSKKSKVN

SEQ ID NO: 49 - protein, 10 aa, Drosophila melanogaster
ILLSKKTKSK

SEQ ID NO: 50 - protein, 10 aa, Arabidopsis thaliana
IQLSKVTKLN

SEQ ID NO: 51 - protein, 10 aa, Dictyostelium discoideum
IKYQKKLGGR

SEQ ID NO: 52 - protein, 10 aa, Homo sapiens
VCRVKAMLTL

SEQ ID NO: 53 - protein, 10 aa, Xenopus tropicalis
VYRVKAMLVL

SEQ ID NO: 54 - protein, 10 aa, Danio rerio
VYRVKAMLTL

SEQ ID NO: 55 - protein, 10 aa, Drosophila melanogaster
VFRVKALLAA

SEQ ID NO: 56 - protein, 10 aa, Dictyostelium discoideum
NMEVKGILAG

SEQ ID NO: 57 - DNA, 12 bp, Homo sapiens
tccccctgctct

SEQ ID NO: 58 - protein, 4 aa, Homo sapiens
SPCS

SEQ ID NO: 59 - DNA, 12 bp, Homo sapiens
tcacgagacttc

SEQ ID NO: 60 - protein, 4 aa, Homo sapiens
SRDF

SEQ ID NO: 61 - DNA, 12 bp, Homo sapiens
tccctctgctct

SEQ ID NO: 62 - protein, 4 aa, Homo sapiens
SLCS

SEQ ID NO: 63 - DNA, 12 bp, Homo sapiens
tcacaagacttc

SEQ ID NO: 64 - protein, 4 aa, Homo sapiens
SQDF

SEQ ID NO: 65 - protein, 1328 aa, Homo sapiens [sequence omitted]

SEQ ID NO: 66 - protein, 1321 aa, Xenopus tropicalis
AGAMAQRILQLAAEGSPERLQEALQGLTEGEVPGGGGASPALSSGRGAASPALEGAGWSGAAHSAAFSFSWQSGVLRRLQVYKHCVSLVESGDLHVGKVSEIIGLLMLEARQLPGHALAELATLFVEVIKRGSLSNGKSLELFSTVLTALSNSKESLAYGKGELNGEEFKKQLINTLCSSKDIPLSGEELQFVVEKVLRMFSKLDLQEIPPLVYQLLLLSAKFSFSDKPYRSADLEVATVPLDQLRHVEGTVILHIVSAINLDQDIGEELIKHLKIFDFLKTSITRSCKDLQILQASKFLQDLCPQQYDVTAVILEVVKNSAFGWDHVTQGLVDLGFSLMESYEPKKSFGGKAAETNLGLSKMPAQQACKLGASILLETFKVHEPIRSDILEQVLNRVLTKAASPVSHFIGSCHGITDAQKIGSRQFLALPSSSSLGRCLGVPGAAGMPKLNPLLLYLSALWWGEALQLGYLLSNIVVSAPLVLQNSSSRVTETFDNLSFLPIDTVQGLLRAVQPLLKVSMSVRDSLILVLQKAIPSRQLDARKAAVAGFLLLLRNFKILGSLTSSQCSQAIGATQVQADVHACYNSAANEAFCLEILGSLRRCLSQQADVRLMLYEGFYDVLRRNSQLASSIMETLLSQIKQYYLPQQDLLPPLKLEGCIMAQGDQIFLQEPLAHLLCCIQHCLAWYKSTMLESVTRRMIKSELEDFELDKSADFSPSSGVGVKNNIYATQVMGICEVLIEYNFKIGNFSKNKFEDVLGLFTCYNKLSEILKEKAGKNKSTLGNRIARSFLSMGPVSTLLTALFRDNAQSHEESLAVLRSSTEPMRYAVSVALQKVQQLEEMGQTDGPDGQNPEKMFQNLCKITRVLLWRYTSIPTAVEESGKKKGKSISLLCLEGLLRIPNTMQQLYAARIPQFLQALDITDGDAEEADINVTEKAAPQIRQFQRSLVNQLSSAEDDFNSKETQLLITILSTLSKLLDPGSQQFLQPLTWTVKICKENALEDLSCCKGLLTLLFSLHVLYKSPVSLLRELAQDIHACLGDIDQDVEIESRSHFAIVNVKTAAPTVCLLVLGQADKVLEEVDWLIKRLTILGSDTSEDSTQASNQTQALEKGVILQLGTLLTVFHELVQTALPAGSCVDSLLRSLSKTYAILTSLIKHYIQACHSTSNTVPGRLEKLVKLSGSHLTPQCYSFITYVQNIHSESLSFAEEKKKKKKEDETAVVSTVMAKVLRDTKPIPNLIFAIEQYEKFLIHLSKKSKVNLMQYMKLSTSRDFRINASMLDSVLQEQNTEDAENEPDNNQSGTAEQPDENQEPQKKRRRK

SEQ ID NO: 67 - protein, 1340 aa, Danio rerio
MDQKILSLAAEDKNEGLQCCLQNLKETELLEIITKHAVKGKECGALLRGIPRGSPCSQDVAVQRRLTVYRHCIQLVESGDLQREVASEIMGLLMLEVHLFGASLIELANLFVDAVKGGSLSNGKSLDLFPTVLSALSSKESLAYGKGELTGEEFKKQLINSLCSSRWDPQSVIFLTSMFRDVSLSVEELQFVVEKVLRMLSKLDLQEMPPLVYQLLLLSAKGSKKNIVEGIITIFNDLDQKQLIQQENSISLDLEDATIPQDQLCHVEGTIILHIVFAIKLDQELGRELLKYLKAGQQGDTSKILCPFSAALLLSVSRIHRFQEQVFDFLKSTILRAYKDHQFHQSSKFLQDLVPQPACVSSILLKTVKNSVYGWDHVTQGLVELGFILMDSFGPKAAFGTKVVETISVNSRTPAQQACQLGSRILLEAFKVHEPIRSEILEQVLNRVITKAANPVTHFLDLLSDIVVSAPLILQNSSSKVTEAFDHLSFLPLTTVQGLLKAVQPLLKISMSMRDSLILVLRKAMFSSQIDARKSAVAGFLLLLRNFKVLGSLSSSQCSQAIGASQIQVDVHMRYNAAANEAFCLEILGSLRRCLSQQADVRLMLYEGFYDVLRRNSQLASSVMQTLLSQLKRYYEPEPDLLPPLKLEGCITAQGDHIFLQEPLAHLLCCIHHCLHWYKSSLLQHRNPDDDDDDDDEDDDHTGCQQDLHDILESITRRMIKCDLEDFELDKSADFSLASGVGVKNNIYAVLVMGICEVLIEYNFIIANFSKSKFEDILGLFKCYSKLSDILKEKAAKGRQPGNSKMARSLISMTFVSTLLTALFRDSTRSHEESLSVLRANMDFMRYSVCVALQKIQQLEETGVTDGPDGQNTEKMFQSICEITRVLMWRYNSIPTAAENPGKKDKGKNISLLCLEGLLRVFNTIQQRYPSKIPQFFTALDALGEEDEEGAREINVTEKAAPQIKQFQRSLINQLSGGEDDPNSKEALLLVSILSTLSRLLAPSSPQFVQMLSWTVKICKETNIEDVQFCKGLMNLLFSLHAQFKSPVSVLRELCQDIHGHLGDIDQDIEVEKQSHFASVSLKTAAPTVTLLVLAQAAKVLEEVDWLIIKLKGLLGSEKLSTEDLTQTSNARLPIEKAIILQLGTLLTACHELVQTALPAGSCTETLLKELAKMYTILTSVVKYYLQICSSHGGQISARLEKLVKLSGSHLTPQCYAFITYVQNIHSESLSLAGEKKKKKKEEASTVTTAKILRDTKPIPNLIFAIEQYEKFLIHLSKKSKVNLMQYMKLSTSRDFRINAATLDAALQEKGSEDEENEPDNEQTVTEEQSQEPKKKRRRK

SEQ ID NO: 68 - protein, 1418 aa, Drosophila melanogaster
MARESILTWMRSLGEKGRFDELELLIQNTPIEKLKETLKSTMTRSEGITYWNYLLRGFNPESKDALAKRFECVRSFMDGLSSTELSYKQTFDLITRLSQDLATFPSEQLAWIVEHCMDGIRQGDAKCVGWKDLLPDALSLLLARPRFLLNGISIDGMEFRDTTVRSIATMQWPASILTPIADMFKTMNLSSGEIVTVLNKFSGALQELSPMELPALCFQLFSMCSTASQLIITLLALEKYFHRNYYKRLFSDMCSNSTNLDSIDLFSDKELREAEETILHHLNYCTMYKLTEKHLAIMLRNFLHMPDVILTPFMLSAIISMTSINRDPESARISHSILLPFLRNVIKNNEERTLADYSVWYRDTLQRKQVDLQQVLTVLIDQNKDGKDVITPGLVHLVFYLLKSQSPKMHTLAITFLTKFIRKRFIFGQGIIKLISEWMIVYQEQNQFSECLTLLSVADTYTVSECMKPIQSVLFHMQWLSGEQSMRMMNFILPLLKISNRVRDALIDVLCKAMSSSGIQKRRMAIYGFCMILKQLNNSNAVRQTSSATSFCTQHSISGYSIMTQNTLGSRSNPQRNFDMLTLEIIGMLRNCLQQQFDIRCSLYENLQRAVELNAKLVPHVLQVIDWHFRSFFDTPSAEDAGDDLDTVFRIRYDQLVSSSDDQHMEIQLKDNLGKLIQFVANCLAVFDRAPSGYDTREMNRLMSFCVDRMVANRLPLEEITPPATHLKCALVLQQLNIIEGIICHLLLKSKPQNNAVCQILPLFNQHCKLLESLKALSDASKKAHKQLKQTAKNNSTSTCNLTLNGVPVKTMCTQPDNIWDLAILDKLLHLLLDDVVAFAAPEKTVLLRSNEPLVRYVLSVTASRVESIRLEPDYKQLAYSKRTFKQLTDITKVIYERCIQRLPELWKNFDMQSAALATQCFSECLKTAHETYSKKFEDFVKSFDLASIKGSRNMEVVYTIQDVLDDFMTEYGTDDSICKNEFVSKLPVYLLESLEILLDHIDYQDRAATESYTWLLNFCSKNEILNSEMGLVHRMLFVQRQKTHLGPFFDSVARQLGHVLGVQNEDAPSESVELTLKSISTVTIESCLQHLYAAIQKQLNDVDYFVTKANNLNYKCQVVPEIDQIYWRGNLDSMDRSICTQIIHISRTLLVLTNVCIPLGSLMDGLMKLLIQHYTCLKSLTKHYISSYTSDTSIENIKSTKFELLLRAVGKQLPANIYELITYIEANTLDEEAQQHTKKRKVQAERAKVLRETRLIPKVIMVIEDFNKHIILLSKKTKSKNRLTDYLHLGTMRDFDIKSTDLRAAIERSISDGRNISTEDSNAEDRDEDEDENENVEEDDDAQSTTYPEDLEEKEDECLVIEKKEERRRRRAPSSEAEDPQPKRKRGRKAAEQLEAEDKEQVEEEQPETTLKLRRNK

SEQ ID NO: 69 - protein, 1475 aa, Arabidopsis thaliana
RPLTDKDIIRLAQYHHCQTGFSLPPYLLSPSSHDSLLSYLKSRSSSPSPSKQVSEYVIALLSLISLSPTTPSLCSLLASLLISYTQIFNSCKIPSDSDSLKTIQLFGTLLRYLHVKEVKSVVDSILSGVTRVITVDEAQVFDLLPVCFDLLRREGKASEIDFVNSAIDRVLSCEWNKALLTKMVSLAKEFSFLDKGRKSELVEKVFVGIKLIDLQDLPSLVYQLLVLASKGFCKREVIGGVVGYFGSKAETRVASVVRQIEGTVLLHVNFAVKQDPSLGQEVVALVKSDLRAFNHFTVAVLFSVARVRKFGESSLGMLRTALLTAYNDYRLSKDCKWLPVELKEESFLHAKLVEKSLLRAVSECRYGREHVVPSVIQFGFMLLESVEEGRSNEFGDSKGVLGIEKLSIQILGTLFEVHDMTRNEIIEQCKFRILSLKCAKSKPIVRLLGYLVQRYELIMLEFVHHLKELLDYFTFMEGHISCFLVSAVIPLIKFSRDLQDYTILVIRKAMFRREDTVRVAATKVITDLILAEKLAKRDSSFTFQDSASQASSSQQTEMSCIVRGNLFTELNGLLQRCLYQQARVKEVVYDGLVKLVLIDPSSGGHVLDFLMPHFLRFFGQDTDFQLGITSCIRVEGDKFTIEEPLDRLLFCVSWILLLQQQNSSDRPSDAAWPCFGFSLSQDNESIAVNNNVWVHSNFVWQQQVGRNLSHEAYSSALVKIRSFLLGKKLGDIVGQSQDTVSASLEEDKRKSYCLIILGIVQVLLNYIIVDLEKQPEGKKGSVRKEIVDLIDLYESLEKDVGKSKQSNVGKRVRFSACNDTDLGNTSINEEREKVPFLATSSIYQLFLLSFKLYSSKSVGTQSGSQDHSHSSPAKTEKSIFSFTLHVCVGHINSSLCMKEENPLKPLVCGDMKVLGPPLLKVVYLLKPEPPLATGQTNKEKKGRKDVDGRKQCLHLALLSLKELLNIYSSGSGLTGLLEDLLAVPASEDATLEECREASKIEDPLIKSIEIFMAKIMKPMITDFIAQNSNDVEEAKTIIFNCTVQILCDIMLKLGNNLPDKFKQRHGSWAHQICRSCETSNTTVAKSVLKLAISFTTSPGDLCIAVEVAQELQNIMGLDKSDTLEVSESYMVINQSTSASVTSCILQSIDSAIVDMDWATKKLKNFYVVSQKNIHLSDDTESTVGSVLEEALYSMAESTVRILSSFVLMNLKESQAAQFLRLAVRFYKQLAQIVKLRIAPKGCKQILPSLKFQKLVELTCKSLTVPLYPFLSEMQKVKVVQAVITLMFLRIQKREVLLLFQIQEQQESVSHNSKGIINKIKQENKCIPDLIFQIEDCERYLIQLSKVTKLNLLRHAKRSTARDFKIIEDSDPPAGGEDGGNQEEETETQNNNIGEDDLRQESDDDGSEEMLSPRNASSVSSPGLDSDKTIAAATEEDDEEEQEQDEGEEESGDNRPKKMARKSWVVEDSDEDSETF

SEQ ID NO: 70 - protein, 1573 aa, Dictyostelium discoideum
IDENNDDLMFSSQNQKKKQKNNNNNNNNNMTSSKLDIDNLYLIFLSMQDDTDEIAKFLTKRIHERKEIFEYIQVVMKSIMSEKDLKEQKELRGKMFEKFLNEFITYDNIVSIDEKQLEQCIDFLLTECDGLSRHTYREIANLIADSISTIRKPPILQLIPRVFEQLSYQSDCKSVVDQEADESFKSSIVDTICKQRWDPLVFSLIVSMFNDMTLTKAHLELVVAKIFKDGRELHLDEYPALMNNVMLLSRQGLKGKILFSICEFFGELDLRYPPDHHGTQDNGLSYIESFVLTQLTLSIRSDPEIAREYLKLNQNPSHPVIGLLMSMSKIPRHKLIAIDCLKSLSNQVVRKGTDNIEQLFLGAVSKNWENVLDPFVNFAISLMDSNGLVITEQIKSLRIVGGKILPELYVRHNSIRDKIIDEIVGRIVMKSNQISQNWFNLLGRIAEEQQFTIRKYLSKLKELFDYLQYYNASTVKQLIGALKPLLLSDGASFLNNVIIVLKKSNFAREVEARASAVTGFIELLKCVSEILLSINNNNNNNNNNNNNNNNNNNNNNNNNNNNNNNNNNNNNNNNNNNRNNNEHLEKLRNFKIEVFSNLRRALTQQCEIRSILYQGFIDLIGFEKGNGMEFADDIFELLNIQSLHYLELSGNNNSPINIDECLELKQNQVCLVEPIDRLIYCIQYCLNHVFTREHEKLYDQFSKEEAKQNPQPSTQQQKQSLFSNSQSRKPTLLTAQQRINVNRKVLDSLKNIKKSSSRIDLEKTFLKFEALMNECRLDSFELRKFGVSNTKAVAASQLLIGVLEALIENSSLQFQPSFGRILALFSLYSDIKNFQSENQAAIKSSSNSNTSKSKKSKKGSKNNDNNGDDDEDDDINDDNTGSDENGENDENDSDNNKKSKSKKTSSSSSKTSSSSSSSSSSSNNVTSNEFPSQKCTQTILKIIYNSNEFNRNNIQHKNFLTYIYTTLSNLLTKLSNSTKSLKKTTDLDSHNKSLRITMEFFKEILPFVYQEFSYQEWITPPKLEKKTDTKKVLVHSLALSCFLTIVQFVNENGNEANLHIVFNNTIDEIKKKEFEKAVLSYKRDHEYYEQQQLQRIAKRSGKSKQQQQNNNRGGNSDDDGDQIMENVEEEEEEELIEPVEPNRESFKPERGPKVIQSISVCIETIIVQHLFPEYYTDLECLFNILTILYSHIQDVQTCFLFLKKISSKHAIESPTLANIILKSIIDMCNSKNSPITETLAKYVLASQGTYKNQSQPVDAYNKPIPPKVLAIINSKTVATAINLLLSNEESVIDRSRERISQFKKDWNSIGKSMSSSNSSNDKIAIDAFNDRNKLASTLNSTCQVLNQLAITKIAGSPHEKFLKLIKKLYTVLTTFINFLIQITTGAITPEIEDLINQSSLLYSYVVDFIEWTNNSLEKDTNNNNNTKKLSKLDLQRMGRESKLLPTITFTLTKYETTVIKYQKKLGGRFILGKHFKEIKFREFFIDTDKLAGIITNDDDGNPVKPPPKKKISKQAPKKSSAGTKRKVRSNDENDDTDGVGDNNDDDDDDDTSPKKKRSVNVSKPKKLYPKYKK

SEQ ID NO: 71 - protein, 195 aa, Homo sapiens
QEVVGALVTHICSGNEAEVDTALDVLLELVVLNPSAMMMNAVFVKGILDYLDNISPQQIRKLFYVLSTLAFSKQNEASSHIQDDMHLVIRKQLSSTVFKYKLIGIIGAVTMAGIMAADRSESPSLTQERANLSDEQCTQVTSLLQLVHSCSEQSPQASALYYDEFANLIQHEKLDPKALEWVGHTICNDFQDAFV

SEQ ID NO: 72 - protein, 192 aa, Danio rerio
QEVVGSLVTHACSGVSGEVDVALKLLCELVSQKPAEMSQFTVFVKGILDYMDNLTSQQIRRLFHLLSRLAFGQEQHGGHIQDDMHIVIRKQLSSTVPKYKRIGIIGAVMMVGSMGACRNKHDGSQGGTLPKETNRQVMALLELVRSCSESSPEAAALCYDELANLLQTCKLDPLVQAWIGKSVLEDFQDDFV

SEQ ID NO: 73 - protein, 196 aa, Gallus gallus
QEVVCALVTHVCSGNETELDISLDVLTDLVILHPSLLLRYATFVKTILDSMQKLNPCQIRKLFYILSTLAFSQRQEGSYIQDDMHMVIRKWLSSSVPNHKQMGIIGAVTMMGSVALKRNEADGGLLERPELSIECDGQLSTLLDLVGFCCEQTPEVLALYYDELANLIEKQKGNLDLQLLDKFGKSLVEDFPNDFV

SEQ ID NO: 74 - protein, 106 aa, Arabidopsis thaliana
VKILGALVTHVGSDNKFEVSSVLEMMTALVKKYAQQLLPFSSHINGISGTCILDYLEGFTIDNLHKTYSQVYEVFSLLALSARASGDSFRSSISNELMMIVRKQLT

SEQ ID NO: 75 - protein, 188 aa, Ciona intestinalis
QETLGFIIEHISATNASSGNTSEADTALDVLREVAQVEVGILLPYASYISDSLTNMDIMNLSQVRKSYRILCYMAFTDSTDAYILGMKDELQMMLRKQLTNLSERYQHVGIIGCLTSLIAMSQSNKKKDSSPATDIQSLFKTLNYLLGNQAHSWSLFYDEICIACKSYGKLPGQLIKLLNKHAQEKLQ

SEQ ID NO: 76 - protein, 217 aa, Anopheles gambiae
RVVLSKIVGFICEMAAKDEMISGLLVLGKLHESHPKEIERSAELLLRILDIAPELSLRQYRPLISVIYAAVIPPRPLDDDAELAAIRDNLEIIVKKQLMCDSKDTKRKGIIGLVQMVYHLSLAPASDDAAELSSSFDSERTIGTVSEIPSATGRTLANLVSTLFLSTNQSPDLLAICYDELAGMLAQPRPKAAPVWEKTFIMWLSDTITMEFQGTFL

SEQ ID NO: 77 - protein, 210 aa, Drosophila melanogaster
KNILKKLLELTCDKSSPHLTTMALELLRELQRKSAKDVQNCATLLIPMLDRISDLSLTQTRVAMDLLCHVAFPDPNLSPCLQLQEQVDMVVKKQLINSIDNIKKQGIIGCVQLIDAMARIANNGVDRDEFIASVENVDSLPDGRGKMAANLIIRTEASIGNSTESLALFFEELATVFNQRNEGTSGCELDNQFIAWACDLVTFRFQASFV

SEQ ID NO: 78 - protein, 183 aa, Caenorhabditis elegans
EKVFTELMKHFSQSETECEAVLDVFFKVIKSTNSSVIQEFSTQIQKCIELIHKLSIPNIKRLFQVMLMMPSKSTEKEATRFEIDEIISRFLLSVSDREKFLGLLALLMKIQVEMGKKHSENQEHLVREGISRCEEAVKSSSALRGAFYEHMRIVLQQHKRTEEIRFLSDWSTDLLKRFKNDFF

SEQ ID NO: 79 - protein, 199 aa, Homo sapiens
QEILEQVLNRVVTRASSPISHFLDLLSNIVMYAPLVLQSCSSKVTEAFDYLSFLPLQTVQRLLKAVQPLLKVSMSMRDCLILVLRKAMFANQLDARKSAVAGFLLLLKNFKVLGSLSSSQCSQSLSVSQVHVDVHSHYNSVANETFCLEIMDSLRRCLSQQADVRLMLYEGFYDVLRRNSQLANSVMQTLLSQLKQFYE

SEQ ID NO: 80 - protein, 199 aa, Tetraodon nigroviridis
GEILEQVLNRLVTKTASPVSHYLDLFSDVVNSAPMIVLESSSKVTETFDHLSYLPLATVQGLLKALQPLLKVSMTLKDALILVLRKAMFSSQLDGRKSAVTGFLLLLKNFKVLGSLASSQCSQAVTASQVQVDVHSRYNAAANEAFCLEILSSLRRCLSQQADVRLLLYEGFYDVLRRNSQLSSSVMQTLLSQVKRYYE

SEQ ID NO: 81 - protein, 208 aa, Ciona intestinalis
KDIIQRVIDQIQSSSDMPAYHFIRLLKEIIKTYPYAFQQCINQLKALLEEMYMMHPYNAHKLLPAIMPLVKNNGALRDSLLVVLRKLLSSRDVNCRISAATGFLLVVQNFPLSEITSLSQISSQSTQSIFSTASTQTQSQLVGSQGRKVNNELLCLEVLQTLSQHCFTQQAEVKIIIYRNLAEVVATNPRLTPYLLDYLLNHLKLYYE

SEQ ID NO: 82 - protein, 197 aa, Arabidopsis thaliana
NEIIEQCKFRILSLKCAKSKPIVRLLGYLVQRYSLIMLEFVHHLKELLDYFTFMEGHISCFLVSAVIPLIKFSRDLQDYTILVIRKAMFRREDTVRVAATKVITDLILAEKLAKRDSSFTFQDSASQASSSQQTEMSCIVRGNLFTELNGLLQRCLYQQARVKEVVYDGLVKLVLIDPSSGGHVLDFLMPHFLRFFG

SEQ ID NO: 83 - protein, 197 aa, Oryza sativa
TEIIEQCKFRILSVKPSQSLPVIRLLGGLVRTHPFQMLEYISHLKELLDYFAFLNDKISIGLINCILPLTKFSRDLKDYIILVIRKAMFKREDAVRIAATNAIVELIIAENKHKRTEANPFQDSSSQPSSSQQPETHLEIGGGLFQELSGLLRRCFMQQARVKEVLYNGLIQIVTSDPSIAENVLDFLWPHFLNYYT

SEQ ID NO: 84 - protein, 206 aa, Drosophila melanogaster
QGIIKLISEWMIVYQEQNQFSECLTLLSVADTYTVSECMKPIQSVLEHMQWLSGEQSMRMMNFILPLLKISNRVRDALIDVLCKAMSSSGIQKRRMAIYGRCMILKQLNNSNAVRQTSSATSFCTQHSISGYSIMTQNTLGSRSNPQRNFDMLTLEIIGMLRNCLQQQFDIRCSLYENLQRAVELNAKLVPHVLQVIDWHFRSFFD

SEQ ID NO: 85 - protein, 207 aa, Aedes aegypti
NGIVAKLKQYLFAELDSMQFSECLNTLSLTSALTLSECTSSINYILEYLLLIDGLHAMRIMTFLFPLIRISHTIRDAFIEVLRKAMYHSEVHTRMMGVFGFCALLKQLKNNNSRRAVLGGSAGNLTQLSISGMSLLSQSTLGRADNPDLHFDMLTLEILGILRRCFTQTVEIRELLYESLGRAVEFNGKLLPHVMQFIDWHFSSYFS

SEQ ID NO: 86 - protein, 207 aa, Anopheles gambiae
SGIIAKIKKYLFADLEESQFNTCLTTLSLTNALTVSECTNTIQFIMDYLLLINGTHAMRFMAFIFPLLRISHTIRDRYIEVLRKAMYNGETSTRIMGVFGFCSLLKQLKNNNARRSIMGSGGGNYTQLTISGMSLLSQAVYGSQNNPNVHFDMLTLEIMGMLRKCFTQTLEVRQMLYEALGRAVEFNTQLLPHVLQFIDWHFESFFG

SEQ ID NO: 87 - protein, 203 aa, Caenorhabditis elegans
GAQLSHLFKSIASSSNNSSALILIDVLRELVRKCSVEILNNSKLIDGLFDYVCRMRKDIAVSLIRCLIPIINTRPQLRNALFKSLKKDLLCESTVGSAVPIVLILLRSVSKRREDGGGGQFDHSMSQSFGTFSTQTLNSMGLKKNVDQTVGLELVGIIKRCLWQPVKTKIALYDGICELATQTSTMLNQFLDMILSHARMIPE